SEQUENCE LISTING (10 sequences)

SEQ ID NO: 1 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 2 - protein, 144 aa, Artificial Sequence
  Description of Artificial Sequence Synthetic polypeptide
QNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - DNA, 30 bp, Artificial Sequence
  Description of Artificial Sequence Synthetic primer
cgggatcccagaatcctcaaattgcggcac

SEQ ID NO: 4 - DNA, 31 bp, Artificial Sequence
  Description of Artificial Sequence Synthetic primer
cggaattctcagagtttgagtaagccaaagg

SEQ ID NO: 5 - protein, 10 aa, Artificial Sequence
  Description of Artificial Sequence Synthetic peptide
  MISC_FEATURE (1)..(10): This sequence may encompass 5-10 residues
HHHHHHHHHH

SEQ ID NO: 6 - protein, 6 aa, Artificial Sequence
  Description of Artificial Sequence Synthetic 6xHis tag
HHHHHH

SEQ ID NO: 7 - protein, 21 aa, Artificial Sequence
  Description of Artificial Sequence Synthetic peptide
QPNDGQSHVGSDNHYSASTTM

SEQ ID NO: 8 - protein, 20 aa, Artificial Sequence
  Description of Artificial Sequence Synthetic peptide
GGGGSGGGGSGGGGSGGGGS

SEQ ID NO: 9 - protein, 4 aa, Artificial Sequence
  Description of Artificial Sequence Synthetic peptide
HDRK

SEQ ID NO: 10 - protein, 4 aa, Artificial Sequence
  Description of Artificial Sequence Synthetic peptide
HDRR

(19) United States
(12) Patent Application Publication
(10) Pub. No.: US 2022/0135649 A1
(43) Pub. Date: May 5, 2022

(54) DOMINANT NEGATIVE CD40L POLYPEPTIDES

(71) Applicant: The Regents of the University of California, Oakland, CA (US)

(72) Inventors: Yoshikazu Takada, Oakland, CA (US); Yoko K Takada, Oakland, CA (US)

(21) Appl. No.: 17/427,518

(22) PCT Filed: Feb. 5, 2020
(86) PCT No.: PCT/US2020/016887

§ 371 Date: Jul. 30, 2021

Related U.S. Application Data
(60) Provisional application No. 62/802,080, filed on Feb. 6, 2019.

(51) Int. Cl. C07K 14/705; A61K 47/60; A61P 17/06; A61P 9/10; A61P 29/00

(52) U.S. Cl. CPC C07K 14/70575; A61K 47/60; A61P 17/06; A61K 38/00; A61P 29/00; C07K 2319/30; C07K 2319/31; A61P 9/10

(57) ABSTRACT

Provided herein are dominant negative CD40L polypeptides, as well as compositions comprising the polypeptides and nucleic acids encoding the polypeptides. Methods for inhibiting CD40/CD40L signaling, inhibiting cell proliferation, and preventing and treating conditions such as inflammatory and immune disorders are also provided herein.

This application is a U.S. National Stage of PCT/US2020/016887, international filing date Feb. 5, 2020, which claims the benefit of priority to U.S. Provisional Patent Application No. 62/802,080, filed Feb. 6, 2019, the contents of which are hereby incorporated by reference in the entirety for all purposes.

### BACKGROUND OF THE INVENTION

### Reference to Submission of a Sequence Listing as a Text File

The Sequence Listing written in file Sequence-Listing_1260769.txt created on Jul. 26, 2021, 5,991 bytes, machine format IBM-PC, MS-Windows operating system, is hereby incorporated by reference in its entirety for all purposes.

### Background of the Invention

CD40 ligand (CD40L) is a key player in chronic autoimmune inflammatory diseases, including systemic lupus erythematosus (SLE), diabetes, and chronic kidney disease. Clinical trials using humanized or chimeric anti-CD40L monoclonal antibodies that block CD40/CD40L interactions were previously undertaken but were halted due to the incidence of thromboembolic events. Therefore, there is a need for new therapeutic agents that target CD40/CD40L signaling and that carry a lower risk of adverse events. The present invention addresses this need and provides related advantages as well.

### BRIEF SUMMARY OF THE INVENTION

In a first aspect, provided herein is an isolated polypeptide comprising the amino acid sequence of SEQ ID NO:1 in which at least one of Y170, H224, G226, and G252 is mutated. In some embodiments, the polypeptide consists of the amino acid sequence of SEQ ID NO:1 in which at least one of Y170, H224, G226, and G252 is mutated. In some embodiments, Y170 is mutated, preferably having the mutation of Y170E. In some embodiments, H224 and G226 are mutated, preferably having the mutations of H224E and G226E. In some embodiments, G252 is mutated, preferably having the mutation of G252E. In some embodiments, the polypeptide suppresses CD40 signaling.

In some embodiments, the polypeptide binds to Δvβ3 integrin or α5β1 integrin with a weaker affinity than a corresponding polypeptide that comprises the amino acid sequence of SEQ ID NO:1. In some embodiments, the polypeptide further comprises a cysteine residue at the N- and/or C-terminus of the amino acid sequence of SEQ ID NO:1. In some embodiments, the polypeptide further comprises one or more polyethylene glycol (PEG) or myristoyl groups. In some embodiments, the polypeptide has an increased half-life in a mammal as compared to a corresponding polypeptide that does not further comprise the one or more PEG or myristoyl groups.

In a second aspect, provided herein is a fusion protein comprising a polypeptide comprising the amino acid sequence of SEQ ID NO:1 in which at least one of Y170, H224, G226, and G252 is mutated and an Fc polypeptide. In some embodiments, Y170 is mutated, preferably having the mutation of Y170E. In some embodiments, H224 and G226 are mutated, preferably having the mutations of H224E and G226E. In some embodiments, G252 is mutated, preferably having the mutation of G252E. In some embodiments, the fusion protein suppresses CD40 signaling. In some embodiments, the fusion protein has an increased half-life in a mammal as compared to a corresponding protein that does not comprise the Fc polypeptide.

In a third aspect, provided herein is a composition comprising a polypeptide or fusion protein of the present invention and a physiologically acceptable carrier.

In a fourth aspect, provided herein is an isolated nucleic acid comprising a polynucleotide sequence that encodes a polypeptide or fusion protein of the present invention.

In a fifth aspect, provided herein is a method for suppressing CD40 signaling in a cell, the method comprising contacting the cell with an effective amount of a polypeptide or a fusion protein of the present invention, a composition of the present invention, or a nucleic acid of the present invention.

In a sixth aspect, provided herein is a method for inhibiting proliferation of a cell, the method comprising contacting the cell with an effective amount of a polypeptide or a fusion protein of the present invention, a composition of the present invention, or a nucleic acid of the present invention. In some embodiments, the cell is a lymphocyte.

In a seventh aspect, provided herein is a method for preventing or treating an inflammatory or immune disorder or cancer in a subject (e.g., a patient) in need thereof, the method comprising administering to the subject an effective amount of a polypeptide or a fusion protein of the present invention, a composition of the present invention, or a nucleic acid of the present invention. In some embodiments, the inflammatory or immune disorder is selected from the group consisting of an autoimmune disorder, systemic lupus erythematosus (SLE), rheumatoid arthritis, atherosclerosis, psoriasis, diabetes, an inflammation- or immune-mediated renal disease, transplant rejection, and a combination thereof.

In methods of the present invention, in some embodiments, the method comprises contacting a population of cells with the polypeptide or fusion protein, the composition, or the nucleic acid. In some embodiments, the cells are mammalian cells. In some embodiments, the cells are located within the body of a mammal. In some embodiments, the subject is a mammal. In some embodiments, the mammal is a human. In some embodiments, the polypeptide, fusion protein, composition, or nucleic acid is administered orally, intraperitoneally, or intravenously.

When nucleic acids are employed in methods of the present invention, in some embodiments, the nucleic acid is an expression cassette comprising a promoter operably linked to a sequence encoding a polypeptide of the present invention. In some embodiments, the promoter is heterologous to the polypeptide-encoding sequence.

Other objects, features, and advantages of the present invention will be apparent to one of skill in the art from the following detailed description and figures.

### BRIEF DESCRIPTION OF THE DRAWINGS

FIG. 1A-1C show that CD40L binds to integrin Δvβ3. FIG. 1A shows the results of experiments in which WT soluble CD40L (KGD deleted, Δ115-117) was immobilized to wells of a 96-well microtiter plate and incubated with soluble Δvβ3 (5 μg/ml) in Tyrode/HEPES buffer (+1 mM MnCl₂ to fully activate Δvβ3) for 1 h, and bound Δvβ3 was quantified using anti-β3 antibody (AV10). FIG. 1B shows a docking simulation between monomeric CD40L that lacks the KGD motif (PDB code 1ALY), and the headpiece of Δvβ3 (PDB code 1L5G), which has an active headpiece conformation. FIG. 1C shows the positions of amino acid residues involved in integrin binding. Notably, the integrin binding sites are located in the trimeric interface of CD40L and are distinct from CD40 binding sites.

FIGS. 2A-2C show that CD40L mutants that are defective in integrin binding are defective in signaling and are dominant-negative. FIG. 2A shows that Y170E, H224E/G226E, and G252E are defective in binding to integrins (Δvβ3+, α5β1). FIG. 2B shows that Y170E, H224E/G226E, and G252E are defective in binding to α5β1. FIG. 2C shows that Y170E and H224E/G226E are defective in inducing proliferation of Ramos B lymphoma cells (CD40+).

FIGS. 3A-3D show that CD154/CD40L allosterically activates integrin Δvβ3. FIG. 3A shows that WT CD40L activates soluble integrin Δvβ3. γC399tr was immobilized to wells and incubated with soluble Δvβ3 (5 μg/ml) in the presence of 1 mM Ca²⁺. Bound Δvβ3 was quantified. FIG. 3B shows that CD40L binds to site 2 peptide. CD40L was immobilized and incubated with site 2 peptide fused to GST. Bound GST was measured. FIG. 3C shows that CD40L mutants defective in binding to site 1 allosterically activate integrin Δvβ3. This indicates that CD40L binds to the two sites in different ways. FIG. 3D shows a docking simulation of CD40L binding to site 2 between CD40L (1ALY.pdb) and the Δvβ3 headpiece (1JV2.pdb, closed-headpiece). This shows that the site 2 binding site of CD40L is distinct from that of site 1.

FIGS. 4A-4C show the positions of site 1 and site 2. Docking simulations of CD40L binding to site 1 and site 2 were performed using open or closed-headpiece as a target. The two docking models were superimposed. This indicates that CD40L binds to site 1 or site 2. Notably, the amino acid residues involved in site 1 and site 2 binding are different.

FIG. 5 shows CD40L-Δvβ3 interaction. Amino acid residues of CD40L involved in integrin binding predicted by docking simulation are described. Notably, several HIGMS1 patients have mutations ate site 1 binding site of CD40L (e.g., Tyr170, Gln174, Thr176, and Ala208).

FIGS. 6A-6C show genetic deletion of integrin β3 and/or β1. Integrin β3 and/or β1 were knocked out from HEK293 cells using CRISPR/Cas9. Control HEK293 and KO cells were stained with anti-β3 or β1. Control HEK293 cells were also stained with control IgG.

FIGS. 7A-7E show that CD40L mutants defective in integrin binding are defective in signaling and suppress CD40L signaling induced by WT CD40L, although they still bind to CD40. FIGS. 7A and 7B shows the results of cell adhesion assays. Wells of 96-well microtiter plates were coated with WT and mutant CD40L and remaining protein binding sites were blocked with BSA. CHO cells and β3-CHO cells were added to the wells in DMEM and incubated for 1 h at 37° C. Bound cells were quantified using endogenous phosphatase activity. FIG. 7C shows binding of CD40L mutants to CD40. The CD40 fragment (residues 21-144) fused to GST (100 μg/ml in PBS) was immobilized to wells of a 96-well microtiter plate and incubated with CD40L mutants. Bound CD40L mutants were measured using anti-His antibodies. FIG. 7D shows that CD40L mutants do not induce CD40 signaling. Ramos cells were incubated with WT CD40L (100 ng/ml) or mutant CD40L for 48 h in RPMI (serum-free) and cell proliferation was determined by MTS assays. FIG. 7E shows that CD40L mutants suppress CD40 signaling induced by WT CD40L. Assays were performed as described in FIG. 7D. WT CD40L (100 ng/ml) and mutant CD40L (500 ng/ml) were added.

FIGS. 8A and 8B shows that two HIGMS1 mutants are defective in integrin binding but intact in CD40 binding. The binding to integrin and CD40 was measured as described above (n=3).

FIGS. 9A-9D show integrin binding to CD40L. FIG. 9A shows binding of soluble integrin Δvβ3 to CD40L. To study whether soluble integrin Δvβ3 binds to CD40L, WT CD40L (residues 118-261) was immobilized in wells of a 96-well microtiter plate and incubated with soluble integrin Δvβ3 in Tyrode/Hepes buffer (+1 mM MnCl₂) for 1 hour, and bound integrin Δvβ3 was quantified using anti-β3 antibody (AV10). FIG. 9B shows that expression of integrin Δvβ3 enhanced binding to CD40L. The effect of integrin Δvβ3 expression on binding to CD40L was studied. Wells of a 96-well microtiter plate were coated with WT CD40L and incubated with CHO cells that expressed recombinant integrin Δvβ3 (β3-CHO) and control CHO cells for 1 hour at 37° C. Bound cells were determined using endogenous phosphatase activity. FIG. 9C shows a docking simulation of CD40L monomer (1ALY.pdb) and the headpiece of integrin Δvβ3 (1L5G.pdb) that was performed using Autodock3. The simulation predicted that CD40L binds to the classical RGD-binding site of integrin Δvβ3 at high affinity (−24 kcal/mol). Tyr-170, His224/Gly226, and Gly252 of CD40L at the integrin binding interface were selected for mutagenesis. FIG. 9D shows that the predicted integrin binding interface of CD40L (including Y170, H224, G226, and G252) is located in the trimeric interface of trimeric CD40L.

FIGS. 10A-10D show that CD40L mutations reduced binding of integrin Δvβ3 and integrin α5β1 to CD40L. The effect of CD40L mutations in the predicted integrin binding site in CD40L on integrin binding was studied. Adhesion of integrin Δvβ3 and integrin α5β1 to CD40L was measured by adhesion assays using Δvβ3-K562 cells (α5β1+, Δvβ3+, CD40-) (FIG. 10A), K562 cells (α5β1+, CD40−) (FIG. 10B), β3-CHO cells (α5β1+, Δvβ3+, CD40−) (FIG. 10C), and CHO cells (α5β1+, CD40−) (FIG. 10D). Wells of a 96-well microtiter plate were coated with WT and mutant CD40L and remaining protein binding sites were blocked with BSA. Cells were added to the wells in Tyrode-HEPES (1 mM MnCl₂) for Δvβ3-K562 and K562 cells, or DMEM for β3-CHO and CHO cells, and incubated for 1 hour at 37° C. Bound cells were quantified using endogenous phosphatase activity. Data are shown as mean+/−SEM (n=3).

FIGS. 11A-11C show the effect of integrin-binding defective mutations on CD40L function. FIG. 11A shows binding of CD40 to CD40L mutants. To study whether CD40L mutants bound to CD40, a CD40 fragment (residues 21-144) fused to GST (100 μg/ml in PBS) was immobilized in wells of a 96-well microtiter plate and incubated with CD40L mutants. Bound CD40L mutants were measured using anti-His antibodies. FIG. 11B shows the effect of CD40L mutants on the proliferation of Ramos cells. The effects of CD40L mutations on the function of CD40L were studied, and it was found that the CD40L mutants were defective in inducing cell proliferation and enhancing cell viability, and suppressed cell viability that is normally enhanced by WT CD40L. Ramos cells were incubated with WT CD40L (100 ng/ml) or mutant CD40L for 48 hours in RPMI (serum-free) and cell viability was determined using an MTS assay. FIG. 11C shows suppression of the anti-apoptotic action of CD40L. To study whether CD40L mutations suppress signals and functions of WT CD40L, WT CD40L (100 ng/ml) and mutant CD40L (500 ng/ml) were added and incubated for 48 hours in RPMI (serum-free). Data are shown as mean+/−SEM (n=5). Cell viability was determined using an MTS assay.

FIGS. 12A-12E show that several CD40L mutations in HIGMS1 in the trimeric interface reduce integrin binding. FIG. 12A shows the positions of HIGMS1 mutations in CD40L. FIG. 12B shows binding of soluble integrin Δvβ3 to immobilized soluble CD40L mutants. Binding assays were performed as described for FIG. 9 above. FIGS. 12C and 12D show binding of HIGMS1 mutations to integrins Δvβ3 and α5β1. Adhesion assays were performed as described for FIG. 10 above. FIG. 12E shows binding of CD40 to HIGMS1 mutants. Binding assays were performed as described for FIG. 11 above.

FIGS. 13A-13C show that CD40L mutants were defective in integrin binding and demonstrated dominant-negative activity. FIG. 13A shows that dnCD40L mutants inhibited wild-type solid-phase CD40L interactions with integrins α5β1 and Δvβ3 on CHO cells, and α5β1 (FIG. 13B). FIG. 13C shows that cell proliferation of human B lymphoma cells (Ramos cells) induced by wild-type CD40L was suppressed by dnCD40L mutants.

### DETAILED DESCRIPTION OF THE INVENTION

### I. Introduction

The present invention is based, in part, on the discovery that integrin Δvβ3 is a receptor for CD40L, and also that integrin αvβ3/α5β1 binding sites are located in the trimeric interface of monomeric CD40L, which is cryptic in trimeric CD40L. The present invention is also based, in part, on the development of new dominant negative CD40L mutant polypeptides that are defective in integrin binding and defective in signaling. The CD40L polypeptides of the present invention are useful for, among other things, the inhibition of CD40/CD40L signaling and cell proliferation, as well as the prevention and treatment of inflammatory disorders, immune disorders, and cancer.

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

The terms “CD40 ligand” and “CD40L” (also known as “CD154”) refer to a protein that is a member of the TNF superfamily of molecules and is encoded by the CD40LG gene in humans. A non-limiting example of a human CD40L mRNA sequence is set forth under NCBI reference number NM_000074. A non-limiting example of a human CD40L protein sequence is set forth under NCBI reference number NP_000065 (SEQ ID NO:1). CD40L is primarily expressed on activated CD4+T lymphocytes but is also found in soluble form. In addition, CD40L is expressed by a wide variety of other cells, including platelets, mast cells, macrophages, basophils, NK cells, B lymphocytes, and non-hematopoietic cells such as smooth muscle cells, endothelial cells, and epithelial cells. In addition to binding to CD40, CD40L binds to integrins such as Δvβ3, α5β1, αIIbβ3, and αMβ2. CD40L functions as a costimulatory molecule and is important on T follicular helper (TFH) cells, where it promotes B cell maturation and activation by binding to CD40 that is located on the B cell surface. Mutations in the CD40LG gene result in an inability to undergo immunoglobulin class switching that is associated with hyper IgM syndrome. CD40L is also important in processes related to the adaptive immune system. Upon binding to CD40 on the surface of macrophages, CD40L expressed on T cells acts as a secondary signal for macrophage activation. Furthermore, CD40L activates endothelial cells, leading to increased reactive oxygen species production, increased chemokine and cytokine production, and increased expression of various adhesion molecules (e.g., E-selectin, ICAM-1, and VCAM-1). A “CD40L polynucleotide” refers to a nucleic acid sequence from the gene encoding the CD40L protein, and may include both the coding and non-coding regions. “CD40L cDNA,” “CD40L mRNA,” “CD40L coding sequence,” and their variations refer to a nucleic acid sequence that encodes a CD40L polypeptide.

The terms “CD40L dominant negative polypeptide,” “CD40L dominant negative mutant,” “CD40L dominant negative mutant polypeptide,” and “CD40L mutant polypeptide” refer to a CD40L antagonist compound in the form of a mutated CD40L polypeptide, or a fragment thereof, which suppresses CD40L-induced CD40 cellular signaling by way of its interaction with integrins (such as integrin Δvβ3 or α5β1) in a manner that imposes an inhibitory or disruptive effect on the specific binding among wild-type CD40L and integrins, thus inhibiting downstream events normally triggered by CD40/CD40L signaling, for example, CD40/CD40L-mediated cellular proliferation. In some embodiments, the dominant negative effect is observed, even though the mutant CD40L polypeptide retains the ability to bind to CD40. In an exemplary CD40L dominant negative mutant, one or more amino acid residues predicted to interact with integrin, e.g., Y170, H224, G226, and G252 residues, are mutated, either by deletion or by substitution with a different amino acid (e.g., the Y170E, H224E/G226E, and G252E mutations), resulting in the mutant having decreased or even abolished capability to bind integrin such as Δvβ3 or α5β1. These CD40L dominant negative mutants can be identified based on their deficiency compared to the wild-type CD40L in decreased integrin binding, as well as in signaling functions (failure to activate or promote cellular proliferation, for example) in test cells (e.g., Ramos cells). In some embodiments, binding affinity for an integrin (e.g., Δvβ3 or α5β1) and/or signaling function is decreased by at least about 10%, 20%, 30%, 40%, 50%, 60%, 70%, 80%, or 90% as compared to wild-type.

A CD40L dominant negative mutant may be initially generated based on a wild-type CD40L amino acid sequence (e.g., SEQ ID NO:1) with certain amino acid residue(s) (e.g., Y170, H224, G226, and/or G252) mutated. In some embodiments, the CD40L dominant negative mutant polypeptide comprises an amino acid sequence having at least about 80% (e.g., at least about 80%, 81%, 82%, 83%, 84%, 85%, 86%, 87%, 88%, 89%, 90%, 91%, 92%, 93%, 94%, 95%, 96%, 97%, 98%, or 99%) identity to SEQ ID NO:1 in which at least one, two, three, or all four of Y170, H224, G226, and G252 are mutated. In some instances, the positions Y170, H224, G226, and/or G252 are not used in calculating the percent identity. In some embodiments, the CD40L dominant negative mutant polypeptide comprises the amino acid sequence set forth in SEQ ID NO:1 in which at least one, two, three, or all four of Y170, H224, G226, and G252 are mutated. In some embodiments, the CD40L dominant negative mutant polypeptide consists of the amino acid sequence set forth in SEQ ID NO:1 in which at least one, two, three, or all four of Y170, H224, G226, and G252 are mutated. In some embodiments, Y170 is mutated. In some embodiments, H224 is mutated. In some embodiments, G226 is mutated. In some embodiments, G252 is mutated. In some embodiments, both H224 and G226 are mutated. In some embodiments, the Y170 mutation is a Y170E mutation. In some embodiments, the H224 mutation is a H224E mutation. In some embodiments, the G226 mutation is a G226E mutation. In some embodiments, the G252 mutation is a G252E mutation. In some embodiments, the CD40L dominant negative mutant polypeptide comprises the amino acid sequence of SEQ ID NO:1 but does not only contain a Y170C mutation, a H224Y mutation, or a G226A mutation.

Furthermore, the CD40L dominant negative mutant polypeptide may further include one or more heterologous amino acid sequences (derived from a source other than CD40L protein) at its N-terminus and/or C-terminus. For example, a CD40L dominant negative mutant may optionally include one or more additional heterologous amino acid sequence(s) of 1, 2, 3, 4, 5, 6, 7, 8, 9, 10, 11, 12, 13, 14, 15, 16, 17, 18, 19, 20, 25, 30, 35, 40, 45, or up to 50 amino acids at the C- and/or N-terminus of the CD40L-derived sequence. In some embodiments, the one or more heterologous amino acid(s) comprise a cysteine residue that is located at the N- and/or C-terminal end and may be used, for example, to attach PEG group(s). Such heterologous peptide sequences can be of a varying nature, for example, any one of the “tags” known and used in the field of recombinant proteins: a peptide tag such as an AviTag, a peptide allowing biotinylation by the enzyme BirA and so the protein can be isolated by streptavidin, a Calmodulin-tag, a peptide bound by the protein calmodulin, a polyglutamate tag, a peptide binding efficiently to anion-exchange resin such as Mono-Q, an E-tag, a peptide recognized by an antibody, a FLAG-tag, a peptide recognized by an antibody, an HA-tag, a peptide recognized by an antibody, a His-tag, 5-10 histidines bound by a nickel or cobalt chelate, a Myc-tag, a short peptide recognized by an antibody, an S-tag, an SBP-tag, a peptide that specifically binds to streptavidin, a Softag 1 for mammalian expression, a Softag 3 for prokaryotic expression, a Strep-tag, a peptide that binds to streptavidin or the modified streptavidin called streptactin (Strep-tag II), a TC tag, a tetracysteine tag that is recognized by FlAsH and ReAsH biarsenical compounds, a V5 tag, a peptide recognized by an antibody, a VSV-tag, a peptide recognized by an antibody, an Xpress tag; or a covalent peptide tags such as an Isopeptag, a peptide that binds covalently to pilin-C protein, a SpyTag, a peptide that binds covalently to SpyCatcher protein; or a protein tag such as a BCCP tag (Biotin Carboxyl Carrier Protein), a protein domain biotinylated by BirA enabling recognition by streptavidin, a Glutathione-S-transferase (GST) tag, a protein that binds to immobilized glutathione, a Green fluorescent protein (GFP) tag, a protein that is spontaneously fluorescent and can be bound by nanobodies, a Maltose binding protein (MBP) tag, a protein that binds to amylose agarose, a Nus-tag, a Thioredoxin-tag, an Fc-tag (derived from immunoglobulin Fc domain, allowing dimerization and solubilization), a tag that can be used for purification on Protein-A Sepharose; as well as other types of tags such as the Ty tag. Furthermore, the CD40L dominant negative mutants may also include one or more D-amino acids or include chemical modifications such as PEGylation, myristoylation, glycosylation, crosslinking, and the like.

In some embodiments, the CD40L dominant negative mutant polypeptide is present as part of a fusion protein, e.g., a fusion protein comprising a CD40L dominant negative mutant polypeptide described herein and an Fc polypeptide. As used herein, the term “Fc polypeptide” refers to the C-terminal region of an immunoglobulin heavy chain polypeptide. An Fc polypeptide typically contains constant region sequences (e.g., the CH2 domain and/or the CH3 domain) and may also contain the hinge region (or a portion thereof). An Fc polypeptide typically does not contain a variable region. In some embodiments, the Fc polypeptide is an IgG1, IgG2, IgG3, or IgG4 Fc polypeptide.

Furthermore, the fusion protein may be labeled, e.g., with a radionuclide. The term “radionuclide” is intended to include any nuclide that exhibits radioactivity. A “nuclide” refers to a type of atom specified by its atomic number, atomic mass, and energy state, such as carbon 14 (¹⁴C). “Radioactivity” refers to the radiation, including alpha particles, beta particles, nucleons, electrons, positrons, neutrinos, and gamma rays, emitted by a radioactive substance. Examples of radionuclides suitable for use in the present invention include, but are not limited to, fluorine 18 (¹⁸F), phosphorus 32 (³²P), scandium 47 (⁴⁷Sc), cobalt 55 (⁵⁵Co), copper 60 (⁶⁰Cu), copper 61 (⁶¹Cu), copper 62 (⁶²Cu), copper 64 (⁶⁴Cu), gallium 66 (⁶⁶Ga), copper 67 (⁶⁷Cu), gallium 67 (⁶⁷Ga), gallium 68 (⁶⁸Ga), rubidium 82 (⁸²Rb), yttrium 86 (⁸⁶Y), yttrium 87 (⁸⁷Y), strontium 89 (⁸⁹Sr), yttrium 90 (⁹⁰Y), rhodium 105 (¹⁰⁵Rh), silver 111 (¹¹¹Ag), indium 111 (¹¹¹In), iodine 124 (¹²⁵I), iodine 125 (¹²⁵I), iodine 131 (¹³¹I), tin 117m (^{117m}Sn), technetium 99m (^{99m}Tc), promethium 149 (¹⁴⁹Pm), samarium 153 (¹⁵³Sm), holmium 166 (¹⁶⁶Ho), lutetium 177 (¹⁷⁷Lu), rhenium 186 (¹⁸⁶Re), rhenium 188 (¹⁸⁸Re), thallium 201 (²⁰¹Tl), astatine 211 (²¹¹At), and bismuth 212 (²¹²Bi) As used herein, the “m” in ^{117m}Sn and ^{99m}Tc stands for the meta state. Additionally, naturally-occurring radioactive elements such as uranium, radium, and thorium, which typically represent mixtures of radioisotopes, are suitable examples of radionuclides. ⁶⁷Cu, ¹³¹L ¹⁷⁷Lu, and ¹⁸⁶Re are beta- and gamma-emitting radionuclides. ²¹²Bi is an alpha- and beta-emitting radionuclide. ²¹¹At is an alpha-emitting radionuclide. ³²P, ⁴⁷Sc, ⁸⁹Sr, ⁹⁰Y, ¹⁰⁵Rh, ¹¹¹Ag, ^{117m}Sn, ¹⁴⁹Pm, ¹⁵³Sm, ¹⁶⁶Ho, and ¹⁸⁸Re are examples of beta-emitting radionuclides. ⁶⁷Ga, ¹¹¹In, ^{99m}Tc, and ²⁰¹Tl are examples of gamma-emitting radionuclides. ⁵⁵Co, ⁶⁰Cu, ⁶¹Cu, ⁶²Cu, ⁶⁶Ga, ⁶⁸Ga, ⁸²Rb, and ⁸⁶Y are examples of positron-emitting radionuclides. ⁶⁴Cu is a beta- and positron-emitting radionuclide.

In some embodiments, a modification such as PEGylation or myristoylation, or fusion to an Fc polypeptide, increases the half-life (e.g., in the body of a subject such as a mammal) of the polypeptide, as compared to a corresponding CD40L dominant negative mutant polypeptide that does not have the modification of that is not fused to the Fc polypeptide. Increased half-life can be due to, for example, increased stability (i.e., the polypeptide is more resistant to degradation and/or metabolism) and/or decreased clearance (e.g., renal clearance). In some embodiments, half-life of the modified (e.g., PEGylated and/or myristoylated) polypeptide is increased by at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, 60-fold, 70-fold, 80-fold, 90-fold, 100-fold, or more.

The term “inflammation” refers to an organism's (e.g., a mammal's) immune response to irritation, toxic substances, pathogens, or other stimuli. The response can involve innate immune components and/or adaptive immunity. Inflammation is generally characterized as either chronic or acute. Acute inflammation can be characterized by, as non-limiting examples, redness, pain, heat, swelling, and/or loss of function due to infiltration of plasma proteins and leukocytes to the affected area. Chronic inflammation can be characterized by, as non-limiting examples, persistent inflammation, tissue destruction, and/or attempts at repair. Monocytes, macrophages, plasma B cells, and other lymphocytes are commonly recruited to the affected area, and angiogenesis and fibrosis can occur, in some instances leading to scar tissue.

The term “inflammatory condition” or “inflammatory disorder” refers to a condition or disorder that is characterized by or involving an inflammatory response, as described above. A list of exemplary inflammatory conditions includes: systemic lupus erythematosus (SLE), diabetes, chronic renal disease, asthma, autoimmune disease, chronic inflammation, chronic prostatitis, glomerulonephritis, hypersensitivities and allergies, skin disorders such as eczema, inflammatory bowel disease, pelvic inflammatory disease, reperfusion injury, rheumatoid arthritis, transplant rejection, and vasculitis.

The term “cancer” refers to any of various malignant neoplasms characterized by the proliferation of anaplastic cells that tend to invade surrounding tissue and metastasize to new body sites. Non-limiting examples of different types of cancer suitable for treatment using the compositions and methods of the present invention include colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (e.g., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “expression cassette” refers to a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter (e.g., a heterologous promoter). “Operably linked” in this context means that two or more genetic elements, such as a polynucleotide coding sequence and a promoter, are placed in relative positions that permit the proper biological functioning of the elements, such as the promoter directing transcription of the coding sequence. Other elements (e.g., heterologous elements) that may be present in an expression cassette include those that enhance transcription (e.g., enhancers) and terminate transcription (e.g., terminators), as well as those that confer certain binding affinity or antigenicity to the recombinant protein produced from the expression cassette.

The term “heterologous,” as used in the context of describing the relative location or position of two elements, such as two polynucleotide sequences (e.g., a promoter and a polypeptide-encoding sequence) or polypeptide sequences (e.g., a first amino acid sequence (such as one set forth in SEQ ID NO:1 with a mutation or mutations) and a second peptide sequence serving as a fusion partner with the first amino acid sequence), means that the two elements are not naturally found in the same relative location or position. Thus, a “heterologous promoter” of a gene refers to a promoter that is not naturally operably linked to that gene. Similarly, a “heterologous polypeptide/amino acid sequence” or “heterologous polynucleotide” to a CD40L amino acid sequence or its encoding sequence is one derived from a non-CD40L origin or derived from CD40L but not naturally connected to the first CD40L-derived sequence (e.g., one set forth in SEQ ID NO:1) in the same fashion.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

The terms “polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “increase” and “decrease” refer to a detectable positive or negative change, respectively, in quantity from a comparison control, e.g., an established standard control (such as an average level of cellular proliferation or apoptosis induced by wild-type CD40L). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

A composition “consisting essentially of a CD40L dominant negative mutant” is one that includes a CD40L mutant that inhibits specific binding among wild-type CD40L and integrin (such as integrin Δvβ3 or α5β1) but no other compounds that contribute significantly to the inhibition of the binding. Such compounds may include inactive excipients, e.g., for formulation or stability of a pharmaceutical composition, or active ingredients that do not significantly contribute to the inhibition of CD40L-integrin binding. Exemplary compositions consisting essentially of a CD40L dominant negative mutant include therapeutics, medicaments, and pharmaceutical compositions.

The term “effective amount” or “therapeutically effective amount” means the amount of a compound that, when administered to a subject or patient for treating a disorder, is sufficient to prevent, reduce the frequency of, or alleviate the symptoms of the disorder. The effective amount will vary depending on a variety of the factors, such as the particular compound used, the disease and its severity, the age, weight, and other factors of the subject to be treated. Amelioration of a symptom of a particular condition by administration of a pharmaceutical composition described herein refers to any lessening, whether permanent or temporary, that can be associated with the administration of the pharmaceutical composition. For example, the amount of a CD40L dominant negative mutant or a fusion protein is considered therapeutically effective for treating a condition involving undesired inflammation, a condition involving an undesired immune response, and/or cancer when treatment results in eliminated symptoms, delayed onset of symptoms, or reduced frequency or severity of symptoms.

The term “subject,” or “subject in need of treatment” refers to an individual who seeks medical attention due to risk of, or actual sufferance from, a condition involving undesirable inflammation, a condition involving an undesirable immune response, and/or cancer cell proliferation. The term subject can include both animals, especially mammals, and humans. Subjects or individuals in need of treatment include those that demonstrate symptoms of an inflammatory disorder, an immune disorder, and/or cancer, or are at risk of later developing these conditions and/or symptoms.

### III. Recombinant Expression of Polypeptides

A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The polynucleotide sequence encoding a polypeptide of interest, e.g., a CD40L dominant negative mutant polypeptide or a fusion protein described herein, and synthetic oligonucleotides can be verified after cloning or subcloning using, e.g., the chain termination method for sequencing double-stranded templates of Wallace et al., Gene 16: 21-26 (1981).

B. Cloning and Subcloning of a Coding Sequence

An example of a polynucleotide sequence encoding human CD40L is set forth under GenBank Accession No. NM_000074. The corresponding amino acid sequence is set forth under GenBank Accession No. NP_000065. Polynucleotide sequences may be obtained from a commercial supplier or by amplification methods such as polymerase chain reaction (PCR).

The rapid progress in the studies of the human genome has made possible a cloning approach where a human DNA sequence database can be searched for any gene segment that has a certain percentage of sequence homology to a known nucleotide sequence. Any DNA sequence so identified can be subsequently obtained by chemical synthesis and/or PCR technique such as the overlap extension method. For a short sequence, completely de novo synthesis may be sufficient; whereas further isolation of a full length coding sequence from a human cDNA or genomic library using a synthetic probe may be necessary to obtain a larger gene.

Alternatively, a polynucleotide sequence encoding a CD40L polypeptide can be isolated from a cDNA or genomic DNA library using standard cloning techniques such as PCR, where homology-based primers can often be derived from a known nucleic acid sequence encoding an CD40L polypeptide. This approach is particularly useful for identifying variants, orthologs, or homologs of CD40L. Most commonly used techniques for this purpose are described in standard texts, e.g., Sambrook and Russell, supra.

cDNA libraries suitable for obtaining a coding sequence for a human CD40L polypeptide may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of a nucleotide sequence by PCR, the segment can be further used as a probe to isolate the full length polynucleotide sequence encoding the gene of interest (e.g., human CD40L) from the cDNA library. A general description of appropriate procedures can be found in Sambrook and Russell, supra. A similar procedure can be followed to obtain a sequence encoding a human CD40L from a human genomic library, which may be commercially available or can be constructed according to various art-recognized methods. Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library.

Upon acquiring a polynucleotide sequence encoding a CD40L sequence, the sequence can be modified and then subcloned into a vector, for instance, an expression vector, so that a recombinant polypeptide (e.g., a CD40L dominant negative mutant polypeptide) can be produced from the resulting construct. Further modifications to the coding sequence, e.g., nucleotide substitutions, may be subsequently made to alter the characteristics of the polypeptide.

C. Modification of a Polynucleotide Coding Sequence

The amino acid sequence of a CD40L polypeptide may be modified in order to achieve, for example, a dominant negative phenotype pertaining to the inhibition of CD40L/CD40-mediated cellular signaling, cell proliferation, etc., as determined by in vitro or in vivo methods known in the field as well as those described herein. Possible modifications to the amino acid sequence also include conservative substitutions, as well as the deletion and/or addition of one or more amino acid residues (e.g., addition of a tag sequence such as 6× His to facilitate purification or identification) at either or both of the N- and C-termini.

A variety of mutation-generating protocols are established and described in the art, and can be readily used to modify a polynucleotide sequence encoding a CD40L polypeptide. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other possible methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

D. Modification of Nucleic Acids for Preferred Codon Usage in a Host Organism

The polynucleotide sequence encoding a CD40L dominant negative mutant polypeptide or fusion protein described herein can be further altered to coincide with the preferred codon usage of a particular host. For example, the preferred codon usage of one strain of bacterial cells can be used to derive a polynucleotide that encodes a CD40L mutant or fusion protein and includes the codons favored by this strain. The frequency of preferred codon usage exhibited by a host cell can be calculated by averaging the frequency of preferred codon usage in a large number of genes expressed by the host cell (e.g., using a calculation service that is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of the CD40L dominant negative mutant polypeptide or fusion protein.

E. Chemical Synthesis of Polypeptides

The amino acid sequence of human CD40L protein has been established (e.g., GenBank Accession No. NP_000065). Polypeptides of known sequences may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal end to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art.

Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (see, Atherton et al., Solid Phase Peptide Synthesis: A Practical Approach, IRL Press (1989), and Bodanszky, Peptide Chemistry, A Practical Textbook, 2nd Ed., Springer-Verlag (1993)).

### IV. Expression and Purification of Recombinant Polypeptides

Following verification of the coding sequence, a polypeptide of interest (e.g., a CD40L dominant negative mutant polypeptide or fusion protein described herein) can be produced using routine techniques in the field of recombinant genetics, relying on the polynucleotide sequences encoding the polypeptide disclosed herein.

A. Expression Systems

To obtain high-level expression of a nucleic acid encoding a polypeptide of interest, one typically subclones the polynucleotide coding sequence into an expression vector that contains a strong promoter to direct transcription, a transcription/translation terminator, and a ribosome binding site for translational initiation. Suitable bacterial promoters are well known in the art and described, e.g., in Sambrook and Russell, supra, and Ausubel et al., supra. Bacterial expression systems for expressing recombinant polypeptides are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the desired polypeptide in host cells. A typical expression cassette thus contains a promoter operably linked to the nucleic acid sequence encoding the polypeptide and signals required for efficient polyadenylation of the transcript, ribosome binding sites, and translation termination. The nucleic acid sequence encoding the desired polypeptide is typically linked to a cleavable signal peptide sequence to promote secretion of the recombinant polypeptide by the transformed cell. Such signal peptides include, among others, the signal peptides from tissue plasminogen activator, insulin, and neuron growth factor, and juvenile hormone esterase of Heliothis virescens. If, however, a recombinant polypeptide is intended to be expressed on the host cell surface, an appropriate anchoring sequence is used in concert with the coding sequence. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the desired polypeptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant polypeptide is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

B. Transfection Methods

Standard transfection methods are used to produce bacterial, mammalian, yeast, insect, or plant cell lines that express large quantities of a recombinant polypeptide, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide.

C. Purification of Recombinantly Produced Polypeptides

Once the expression of a recombinant polypeptide in transfected host cells is confirmed, e.g., by an immunological assay, the host cells are then cultured in an appropriate scale for the purpose of purifying the recombinant polypeptide.

1. Purification of Recombinantly Produced Polypeptide from Bacteria

When desired polypeptides are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

2. Standard Protein Separation Techniques for Purification

When a recombinant polypeptide is expressed in host cells in a soluble form, its purification can follow the standard protein purification procedure described below. This standard purification procedure is also suitable for purifying polypeptides obtained from chemical synthesis.

i. Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

ii. Size Differential Filtration

Based on a calculated molecular weight, a protein of a particular size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

iii. Column Chromatography

Proteins of interest (such as a CD40L dominant negative mutant polypeptide or fusion protein described herein) can also be separated from other proteins on the basis of their size, net surface charge, hydrophobicity, or affinity for ligands. In addition, antibodies raised against a CD40L mutant or Fc polypeptide can be conjugated to column matrices and the corresponding polypeptide immunopurified. All of these methods are well known in the art.

It will be apparent to one of skill that chromatographic techniques can be performed at any scale and using equipment from many different manufacturers (e.g., Pharmacia Biotech).

### V. Conditions Involving Inflammation, Immune Responses and Cell Proliferation

CD40L dominant negative mutant CD40L polypeptides, fusion proteins (i.e., comprising dominant negative mutant CD40L polypeptides), nucleic acids encoding said polypeptides, and other compositions (e.g., pharmaceutical compositions) of the present invention can be used to inhibit CD40 signaling in a cell, inhibit cell proliferation (e.g., of a lymphocyte such as a B lymphocyte), and/or for the prevention and treatment of a number of inflammatory and/or immune disorders (e.g., in a subject), as well as cancer. Non-limiting examples of suitable inflammatory and/or immune disorders include autoimmune disorders, systemic lupus erythematosus (SLE), non-rheumatoid arthritis, rheumatoid arthritis, atherosclerosis, psoriasis, diabetes, inflammation- or immune-mediated renal disease, transplant rejection, Alzheimer's disease, multiple sclerosis, asthma, adult respiratory distress syndrome (ARS), anaphylactic shock, gout, and combinations thereof.

In some embodiments, methods for inhibiting CD40 signaling, inhibiting cellular proliferation, and/or preventing or treating a disorder such as an inflammatory disorder, immune disorder, and/or cancer comprise contacting a cell with an effective amount of a CD40L dominant negative mutant polypeptide, a nucleic acid encoding a dominant negative mutant CD40L polypeptide, fusion protein, or other composition (e.g., pharmaceutical composition) of the present invention. In some embodiments, a population of cells are contacted. In some embodiments, the cell is located within the body of a subject, such as a mammal (e.g., a human). In some embodiments, the methods comprising administering to a subject (e.g., a mammal such as a human) in need thereof (e.g., a patient in need thereof) an effective amount of a CD40L dominant negative mutant polypeptide, a fusion protein, a nucleic acid encoding a dominant negative mutant CD40L polypeptide or fusion protein, or other composition (e.g., pharmaceutical composition) of the present invention.

Identification and diagnosis of conditions involving inflammation, undesirable immune responses, or undesirable cell proliferation, as well as methods of monitoring the effectiveness of a therapeutic regimen as described herein, are included in the present invention. As explained above, inflammation can be characterized by redness, swelling, pain, and/or loss of function. However, symptoms vary among tissues, so that some inflammatory conditions are not easily detectable (e.g., atherosclerosis). Undesirable cell proliferation, on the other hand, is often determined by way of detecting a benign or malignant growth, including an abnormal expansion of a particular cell or tissue type, such as various types of tumors and cancers.

Although the inflammatory and immune responses can play a role in the healing process by destroying, diluting, and isolating injurious agents and stimulating repair of the affected tissue, inflammatory and immune responses can also be harmful. For example, inflammation results in leakage of plasma from the blood vessels. Although this leakage can have beneficial effects, it causes pain and when uncontrolled can lead to loss of function and death (such as adult respiratory distress syndrome). Anaphylactic shock, arthritis, and gout, in addition to other disorders listed above, are among the conditions that are characterized by uncontrolled or inappropriate inflammation. Furthermore, immune responses can be harmful when the immune system targets normal healthy tissues (e.g., autoimmune disorders), resulting in, for example, pain, swelling, loss of normal tissue or organ function, or even death. In addition, autoimmune disorders (e.g., diabetes, SLE, renal disease) can affect multiple tissues or organ systems, and can also result in secondary pathological conditions. Also, in some cases it is necessary to inhibit what may be otherwise a desired immune response, for example in the scenario where a subject receives a tissue or organ transplant, and it is necessary to inhibit the immune response in order to decrease or prevent rejection of the transplanted tissue or organ by the recipient.

On a cellular level, an inflammatory response is typically initiated by endothelial cells producing molecules that attract and detain inflammatory cells (e.g., myeloid cells such as neutrophils, eosinophils, and basophils) at the site of injury or irritation. The inflammatory cells then are transported through the endothelial barrier into the surrounding tissue. The result is accumulation of inflammatory cells, in particular neutrophils. Such accumulation is easily detectable by one of skill.

Adaptive immune cells (e.g., T and B cells) are involved in immune responses and often involved in inflammatory conditions. These cells release cytokines and antibodies in response to a source of irritation, the presence of an antigen, or other signal. Thus, an immune and/or inflammatory response can also be detected by detecting a change in the level of cytokines, e.g., in a localized region of irritation or in the serum or plasma of an individual. It will be appreciated by those of skill in the art that signs and symptoms associated with these disorders can be detected in an individual for the purposes of diagnosis. Further, a subject undergoing therapy for an inflammatory or immune condition or cancer can be monitored, for instance, by detecting any changes in severity of the signs or symptoms.

### VI. Pharmaceutical Compositions and Administration

The present invention also provides pharmaceutical compositions comprising an effective amount of a CD40L dominant negative mutant polypeptide and/or a fusion protein (i.e., comprising a CD40L dominant negative mutant polypeptide) for inhibiting a pro-inflammatory signal, a pro-proliferation signal, or a pro-immune signal, therefore useful in both prophylactic and therapeutic applications designed for various diseases and conditions involving undesired inflammation, cell proliferation, and/or undesired immune response. Pharmaceutical compositions of the invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990).

The polypeptides, fusion proteins, and compositions (e.g., pharmaceutical compositions) of the present invention can be administered (e.g., to a mammal, such as a human) by various routes, e.g., oral, subcutaneous, transdermal, intramuscular, intravenous, intraperitoneal, mucosal, topical, and/or buccal. The routes of administering the pharmaceutical compositions include systemic or local delivery to a subject suffering from a condition exacerbated by inflammation at daily doses of about 0.01-5000 mg, preferably 5-500 mg, of a CD40L mutant polypeptide or fusion protein for a 70 kg adult human per day. The appropriate dose may be administered in a single daily dose or as divided doses presented at appropriate intervals, for example as two, three, four, or more subdoses per day.

For preparing pharmaceutical compositions containing a CD40L mutant polypeptide and/or a fusion protein (i.e., that comprises a dominant negative CD40L mutant polypeptide), inert and physiologically or pharmaceutically acceptable carriers are typically used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., a CD40L mutant polypeptide or fusion protein (i.e., that comprises a dominant negative CD40L mutant polypeptide). In tablets, the active ingredient (the mutant polypeptide) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of a CD40L mutant polypeptide or fusion protein (i.e., that comprises a dominant negative CD40L mutant polypeptide) with encapsulating material as a carrier providing a capsule in which the mutant (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., a CD40L mutant polypeptide or fusion protein (i.e., that comprises a dominant negative CD40L mutant polypeptide)) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., a CD40L mutant polypeptide or fusion protein (i.e., that comprises a dominant negative CD40L mutant polypeptide)) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing the CD40L mutant polypeptide or fusion protein can be administered for prophylactic and/or therapeutic treatments. In therapeutic applications, compositions are administered to a patient already suffering from a condition that may be exacerbated by an undesirable inflammatory or immune reaction/cell proliferation in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 0.1 mg to about 2,000 mg of the mutant polypeptide per day for a 70 kg patient, with dosages of from about 5 mg to about 500 mg of the mutant polypeptide per day for a 70 kg patient being more commonly used.

In prophylactic applications, pharmaceutical compositions containing a CD40L mutant polypeptide or fusion protein (i.e., that comprises a dominant negative CD40L mutant polypeptide) are administered to a patient susceptible to or otherwise at risk of developing a disease or condition involving an undesirable inflammatory response, undesirable immune response, cell proliferation, and/or cancer in an amount sufficient to delay or prevent the onset of the symptoms. Such an amount is defined to be a “prophylactically effective dose.” In this use, the precise amounts of the inhibitor again depend on the patient's state of health and weight, but generally range from about 0.1 mg to about 2,000 mg of the mutant polypeptide for a 70 kg patient per day, more commonly from about 5 mg to about 500 mg for a 70 kg patient per day.

Single or multiple administrations of the compositions can be carried out with dose levels and pattern being selected by the treating physician. In any event, the pharmaceutical formulations should provide a quantity of a compound sufficient to effectively inhibit the undesirable inflammatory or immune response/cellular proliferation/cancer mediated by CD40L/CD40 signaling in the patient, either therapeutically or prophylactically.

### VII. Therapeutic Applications Using Nucleic Acids

In some aspects, provided herein is an isolated nucleic acid that encodes a CD40L dominant negative mutant polypeptide (e.g., a CD40L dominant negative polypeptide comprising the amino acid sequence of SEQ ID NO:1 in which at least one of Y170, H224, G226, and G252 is mutated) or a fusion protein (i.e., that comprises a dominant negative CD40L mutant polypeptide). Furthermore, a variety of inflammatory disorders, immune disorders, conditions associated with undesirable cell proliferation, and cancers can be treated by therapeutic approaches that involve introducing into a cell a nucleic acid encoding a CD40L dominant negative mutant polypeptide (e.g., a CD40L dominant negative polypeptide comprising the amino acid sequence of SEQ ID NO:1 in which at least one of Y170, H224, G226, and G252 is mutated) or a fusion protein (i.e., that comprises a dominant negative CD40L mutant polypeptide) such that the expression of the mutant leads to reduced or abolished CD40L/CD40 signaling in the cell. Those amenable to treatment by this approach include a broad spectrum of conditions involving undesirable inflammation and/or cell proliferation. For discussions on the application of gene therapy towards the treatment of genetic as well as acquired diseases, see, Miller Nature 357:455-460 (1992); and Mulligan Science 260:926-932 (1993).

A. Vectors for Nucleic Acid Delivery

For delivery to a cell or organism, a nucleic acid encoding a CD40L dominant negative mutant polypeptide (e.g., a CD40L dominant negative polypeptide comprising the amino acid sequence of SEQ ID NO:1 in which at least one of Y170, H224, G226, and G252 is mutated) or a fusion protein (i.e., that comprises a dominant negative CD40L mutant polypeptide) of the invention can be incorporated into a vector. Examples of vectors used for such purposes include expression plasmids capable of directing the expression of the CD40L mutants or fusion proteins in the target cell. In other instances, the vector is a viral vector system wherein the polynucleotide is incorporated into a viral genome that is capable of transfecting the target cell. In a preferred embodiment, the inhibitory nucleic acid can be operably linked to expression and control sequences that can direct transcription of sequence in the desired target host cells. Thus, one can achieve reduced CD40/CD40L signaling under appropriate conditions in the target cell.

B. Gene Delivery Systems

As used herein, a “gene delivery system” refers to any means for the delivery of a nucleic acid encoding a CD40L dominant negative mutant polypeptide (e.g., a CD40L dominant negative polypeptide comprising the amino acid sequence of SEQ ID NO:1 in which at least one of Y170, H224, G226, and G252 is mutated) or a fusion protein (i.e., that comprises a dominant negative CD40L mutant polypeptide) of the invention to a target cell. Viral vector systems useful in the introduction and expression of a nucleic acid include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the nucleic acid is inserted into such vectors to allow packaging of the gene construct, typically with accompanying viral DNA, followed by infection of a sensitive host cell and expression of the gene of interest.

Similarly, viral envelopes used for packaging gene constructs that include the nucleic acid can be modified by the addition of receptor ligands or antibodies specific for a receptor to permit receptor-mediated endocytosis into specific cells (see, e.g., WO 93/20221, WO 93/14188, and WO 94/06923).

Retroviral vectors may also be useful for introducing the nucleic acid of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild-type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleic acid sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, the inhibitory nucleic acid, thus eliminating or reducing unwanted inflammatory conditions.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

C. Pharmaceutical Formulations

When used for pharmaceutical purposes, the nucleic acid encoding a CD40L dominant negative mutant polypeptide (e.g., a CD40L dominant negative polypeptide comprising the amino acid sequence of SEQ ID NO:1 in which at least one of Y170, H224, G226, and G252 is mutated) or a fusion protein (i.e., that comprises a dominant negative CD40L mutant polypeptide) is generally formulated in a physiologically acceptable carrier such as a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can further include a stabilizer, an enhancer, and/or other pharmaceutically acceptable carriers or vehicles. A pharmaceutically acceptable carrier can contain a physiologically acceptable compound that acts, for example, to stabilize the inhibitory nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

D. Administration of Formulations

The formulations containing a nucleic acid can be delivered to any tissue or organ using any delivery method known to the ordinarily skilled artisan. In some embodiments of the invention, the nucleic acid encoding a CD40L dominant negative mutant polypeptide (e.g., a CD40L dominant negative polypeptide comprising the amino acid sequence of SEQ ID NO:1 in which at least one of Y170, H224, G226, and G252 is mutated) or a fusion protein (i.e., that comprises a dominant negative CD40L mutant polypeptide) is formulated for administration (e.g., to a mammal) by an oral, intraperitoneal, intravenous, intramuscular, subcutaneous, mucosal, topical, and/or buccal route. In some embodiments, the nucleic acid is prepared as a mucoadhesive gel or topical gel formulation. Exemplary permeation enhancing compositions, polymer matrices, and mucoadhesive gel preparations for transdermal delivery are disclosed in U.S. Pat. No. 5,346,701.

The formulations containing the nucleic acid are typically administered to a cell. The cell can be provided as part of a tissue or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acid is introduced into cells by such methods as microinjection, calcium phosphate precipitation, liposome fusion, ultrasound, electroporation, or biolistics. In further embodiments, the nucleic acid is taken up directly by the tissue of interest.

In some embodiments of the invention, the nucleic acid is administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. To practice the present invention, doses ranging from about 10 ng-1 g, 100 ng-100 mg, 1 μg-10 mg, or 30-300 μg inhibitory nucleic acid per patient are typical. Doses generally range between about 0.01 and about 50 mg per kilogram of body weight, preferably between about 0.1 and about 5 mg/kg of body weight or about 10⁸-10¹⁰ or 10¹² viral particles per injection. In general, the dose equivalent of a naked nucleic acid from a vector is from about 1-100 μg for a typical 70 kg patient, and doses of vectors which include a retroviral particle are calculated to yield an equivalent amount of an inhibitory nucleic acid.

### VIII. KITS

The invention also provides kits for suppressing CD40/CD40L-induced cellular signaling or treating a condition involving undesirable inflammatory and/or immune responses and/or cell proliferation including cancer cell proliferation by inhibiting the specific binding between CD40L and integrin according to the method of the present invention. The kits typically include a first container that contains a pharmaceutical composition having an effective amount of a CD40L dominant negative mutant polypeptide or a fusion protein (i.e., that comprises a dominant negative CD40L mutant polypeptide), optionally with a second container containing an anti-inflammatory, immunosuppressive, or anti-cancer agent. In some cases, the kits will also include informational material containing instructions on how to dispense the pharmaceutical composition, including description of the type of patients who may be treated (e.g., a person suffering from an inflammatory disorder, immune disorder, or cancer or at risk of developing such a disorder or cancer), the schedule (e.g., dose and frequency of administration) and route of administration, and the like.

### IX. Examples

The present invention will be described in greater detail by way of specific examples. The following examples are offered for illustrative purposes only, and are not intended to limit the invention in any manner.

### Example 1. Development of CD40L Mutant Polypeptides and Validation in a Psoriasis Model

### Abstract

CD40 ligand (CD40L/CD154) plays key roles in immune regulation through binding to its receptor CD40, and is a major target in inflammatory diseases. Earlier development of anti-CD40L antibodies was halted because they induced thromboembolisms in clinical trials. We recently discovered that several growth factors (e.g., FGF) directly bind to integrins and form a ternary complex (e.g., integrin-FGF-FGFR). These findings demonstrate that integrins are common co-receptors of growth factor signaling (ternary complex model). The FGF mutants that are defective in integrin binding are also defective in signaling functions and act as antagonists (dominant-negative). This model may be applied to CD40L, since CD40L is known to bind to integrins (e.g., α5β1) and CD40 and α5β1 can simultaneously bind to CD40L. The work described here examines CD40L signaling mediation by ternary complex formation with integrin α5β1 and CD40, and tests the ability of CD40L mutants defective in integrin binding to act as antagonists. Preliminary studies found that (a) CD40L binds to integrin Δvβ3 (a newly identified CD40L receptor), (b) CD40L mutants defective in binding to Δvβ3 and α5β1 act as antagonists to CD40L, and (c) the Δvβ3 and α5β1-binding site is located in the CD40L-CD40L trimeric interface, demonstrating that the integrin-binding site is cryptic in trimeric CD40L. Since amino acid residues of CD40L involved in integrin binding overlap with those involved in Hyper IgM syndrome (HIGMS1), which is caused by genetic defects in CD40L and induces a defect in immunoglobulin class switching, we explore the relationship between the loss of integrin binding and HIGMS1. We previously reported that several integrin ligands allosterically activate integrins by binding to the allosteric site of integrin (site 2), which we recently discovered. CD40L allosterically activates Δvβ3 by binding to site 2. Since mutations of several amino acid residues of CD40L involved in site 2 binding also induce HIGMS1, we understand that CD40L binding to integrin site 2 is also involved in CD40L signaling. The goal of experiments described in this example is to further explore the role of integrins in CD40/CD40L signaling and develop potential therapeutics for inflammation. In particular, the aims are to: 1) study the role of integrins in CD40L signaling. A) Study how HIGMS1 and the loss of integrin binding are related by testing the ability of HIGMS1 mutations to affect integrin binding. This identifies a cause of HIGMS1. B) Study how monomeric or trimeric CD40L induces CD40L/CD40 signaling. C) Study how leukocyte integrins interact with CD40L. 2). Determine the role of integrin outside-in signaling in CD40L signaling. A) Study how CD40L induces integrin-CD40L-CD40 ternary complex formation. B) Study the role of the integrin outside-in signaling in CD40L signaling. C) Characterize CD40L-induced allosteric activation of integrins. 3). Development of dominant-negative CD40L mutants as therapeutics. A) Study how CD40L mutants defective in integrin binding are dominant-negative (dnCD40L). B) Study the effect of CD40L mutants in a mouse model of skin inflammation in psoriasis.

### Specific Aims

CD40-ligand (CD40L/CD154) plays key roles in immune regulation through binding to its receptor CD40 and its levels markedly increase in certain pathologic conditions (e.g., SLE) (1). In addition, CD40L is a major target in inflammatory diseases. The mechanism of interaction between CD40 and CD40L, however, is still not fully elucidated, and the previous therapeutic development of anti-CD40L antibodies was halted because of the induction of thromboembolism in clinical trials. We recently discovered that several growth factors (e.g., FGF, IGF) directly bind to integrins and form integrin-growth factor-growth factor receptor ternary complexes (2-8). Integrins are common co-receptors of growth factor signaling (ternary complex model). In addition, growth factor mutants that are defective in integrin binding are also defective in signaling functions and act as antagonists (dominant-negative) (5-7). It has been reported that CD40L binds to integrins (e.g., α5β1) and that CD40 and α5β1 can simultaneously bind to CD40L. Thusly, CD40L/CD40 signaling fits well with the ternary complex model. Here, we examine CD40L signaling mediation by ternary complex with integrin α5β1 and CD40, and investigate how CD40L mutants defective in integrin binding act as antagonists. Preliminary studies found that (a) integrin Δvβ3 is a newly identified co-receptor for CD40/CD40L signaling, (b) the Δvβ3 and α5β1-binding site is located in the CD40L-CD40L trimeric interface, demonstrating that the integrin-binding site is not exposed in trimeric CD40L, and (c) the amino acid residue positions of CD40L involved in integrin binding overlap with those involved in the Hyper IgM syndrome type 1 (HIGMS1), which is caused by genetic defect in CD40L and induces the defect in immunoglobulin class switching (9). Thus, the loss of integrin binding is related to HIGMS1. We previously reported that several integrin ligands allosterically activate integrins by binding to the allosteric site of integrin (site 2), which we recently discovered. In our preliminary studies, CD40L allosterically activated Δvβ3 by binding to site 2. The observation that mutations in several amino acid residues of CD40L are involved in site 2 binding and cause HIGMS1, is consistent with CD40L binding to site 2 and that resulting activation of integrins are involved in CD40L signaling. We will explore how CD40L mutants defective in binding to Δvβ3 and α5β1 act as antagonists to CD40L. The goal of the work described in this example is to further explore the role of integrins in CD40/CD40L signaling and develop potential therapeutics for inflammation.

### 1. Study the Role of Integrins in CD40L Signaling.

A) Explore the relationship between HIGMS1 and the loss of integrin binding by testing the ability of HIGMS1 mutations to affect integrin binding. Many, if not all, HIGMS1 mutations affect integrin binding, indicating that the ability to bind to integrins is required for CD40L signaling.

B) Study how monomeric or trimeric CD40L induces CD40L/CD40 signaling. The observations that the integrin-binding site is cryptic in trimeric CD40L and that the predicted integrin-binding interface of CD40L overlaps with the positions of HIGMS1 mutations is consistent with monomeric CD40L being active. We first study the activity of locked trimeric CD40L (by intermolecular disulfide linkage).

C) Study how leukocyte integrins interact with CD40L. The fact that CD40L is involved in immune regulation is consistent with leukocyte-specific integrin α4β1 playing a role in CD40L signaling or being activated by CD40L. This is studied using recombinant integrins.

### 2. Further Elucidate the Role of Integrin Outside-In Signaling in CD40L Signaling.

A) Study how CD40L induces integrin-CD40L-CD40 ternary complex formation. Co-immunoprecipitation or pull-down assays are used to establish the ternary complex formation. We will examine integrin Δvβ3 (and other integrins) forming ternary complexes with CD40 and CD40L.

B) Further elucidate the role of the integrin outside-in signaling in CD40L signaling. Integrin β1 or β3 cytoplasmic mutations that block outside-in signaling from the extracellular matrix are used. We examine the role that this integrin signaling pathway plays in CD40L signaling as well.

C) Characterize CD40L-induced allosteric activation of integrins. The observation that amino acid residues of CD40L for site 2 binding are also involved in HIGMS1 is consistent with the model that site 2 binding is also involved in CD40L signaling.

### 3. Development of Dominant-Negative CD40L Mutants as Potential Therapeutics.

A) Further identify the mechanisms of how CD40L mutants defective in integrin binding are dominant-negative (dnCD40L). An NF-kB reporter system is used to efficiently detect CD40L signaling.

B) Study the effect of dnCD40L mutants in a mouse model of skin inflammation in psoriasis.

This will yield further insight into the mechanisms of CD40L/CD40 signaling and identify the CD40L mutants that act as antagonists.

### Significance

Direct integrin binding and ternary complex formation is required for several growth factors. The finding that integrin antagonists inhibit insulin-like growth factor (IGF1) and fibroblast growth factor-2 (basic FGF, FGF2) signaling (10,11) demonstrated that integrins are involved in growth factor signaling through crosstalk. However, current models of integrin-growth factor crosstalk propose that integrins contribute to growth factor signaling almost exclusively through integrin binding to the extracellular matrix (ECM), and growth factors only bind to their cognate receptors and two independent signals merge inside the cells (12-14). We have reported that several cytokines (FGF1, IGF1, neuregulin-1, and fractalkine) directly bind to integrins and that this interaction leads to ternary complex formation (integrin-growth factor-receptor tyrosine kinase), which is required for their signaling functions (2-8). It is notable that the ternary complex formation is seen in several different growth factors. Previously, we found that direct binding of integrins to IL-1β plays a critical role in IL-1β signaling (15). This indicates that crosstalk between integrins and growth factor receptors through direct integrin binding is a common mechanism in growth factor signaling. Mutants defective in integrin binding to these growth factors are functionally defective and suppress signaling induced by WT growth factor (dominant-negative antagonists) (5-7).

CD40L is a key immunomodulatory factor and a major therapeutic target. CD40 is a cell surface receptor that belongs to the tumor necrosis factor-R (TNF-R) family, and was first identified and functionally characterized on B lymphocytes (16). Its critical role in T cell-dependent humoral immune responses was demonstrated by patients with the hyper-IgM syndrome type 1 (HIGMS1), as well as by gene targeting in mice. However, in recent years it has become clear that CD40 is expressed much more broadly, including expression on monocytes, dendritic cells, endothelial cells, and epithelial cells. In addition, CD40L is also expressed more widely than on activated CD4+ T cells alone (17). Therefore, it is now understood that CD40-CD40L interactions play a more general role in immune regulation.

CD40L is a type II protein ligand member of the tumor necrosis factor (TNF) superfamily that, via interaction with CD40, is a key immunomodulatory factor responsible for modulating nearly all aspects of the adaptive immune response. CD40L is expressed as a transmembrane form and released as a soluble form (sCD40L) by proteolytic cleavage. CD40L/CD40 interaction is required for enhancing antigen presenting functions of dendritic cells, macrophages, and B cells; maturation of humoral responses; and enhancement of effector T cell responses (18). Additional functions of CD40L include the initiation of inflammatory and procoagulatory responses in vascular endothelial cells (19-21). CD40L is a key player in chronic autoimmune inflammatory diseases, including systemic lupus erythematosus (SLE), diabetes, and chronic kidney disease (22, 23). Based on striking efficacy in preclinical models, clinical trials using humanized or chimeric anti-CD40L monoclonal antibodies blocking CD40/CD40L interactions were undertaken in the early 2000s. However, progress was halted due to the incidence of thromboembolic events in clinical trials.

It has been understood that trimeric CD40L is biologically active, but monomeric CD40L is not. CD40L levels markedly increase in certain pathologic conditions (SLE and RA), but CD40L exists mainly in a monomeric form (1).

CD40L simultaneously binds to integrin α5β1 and CD40, but how integrins and CD40 work together can be further elucidated. The contribution of integrins to CD40L/CD40 signaling has been largely ignored (24, 25). It has been reported that CD40L stabilizes arterial thrombi through binding to integrin αIIbβ3 (26). αIIbβ3 recognizes the KGD motif at the N-terminus of CD40L (residues 115-117 of CD40L). It has also been reported that CD40L binds to integrin α5β1 and transduces signals through this integrin in a CD40- and αIIbβ3-independent manner. It has been shown that CD40 and integrin α5β1 can bind to CD40L simultaneously (27). These integrins recognize CD40L differently. α5β1 does not require the KGD motif, and its binding site in CD40L can be more precisely determined. Mutations of the CD40-binding site (Y145A, R203A, or Y145A/R203A double mutant) did not affect α5β1-CD40L interaction (27), indicating that α5β1 and CD40 can co-exist on CD40L.

Integrin Δvβ3 is a novel CD40L receptor, and the integrin-binding site in CD40L is located at the trimeric interphase. In our preliminary studies, we identified integrin Δvβ3 as a novel receptor for CD40L and found that Δvβ3 does not recognize the N-terminal KGD motif of CD40L. (In a previous study, it has been reported that Δvβ3 does not bind to CD40L, but we found that Δvβ3 was not activated in that study (27). We identified an integrin binding site in CD40L using docking simulations and by introducing mutations in the predicted integrin binding site in CD40L. Notably, we found that the Δvβ3 binding site is located in the trimerization interface of CD40L, indicating that the Δvβ3-binding site is not exposed in the CD40L trimer, but exposed in the monomer. This indicates that CD40L monomer plays a role in CD40L signaling. We address this in subaim #1.

CD40L allosterically activates integrins by binding to the allosteric ligand binding site of integrins (site 2) that was recently identified. Integrins are generally believed to be activated by inside-out signaling. We recently, however, discovered that integrins can be activated in an allosteric mechanism. We recently identified two integrin ligands, fractalkine/CX3CL1 and secreted phospholipase A2 type IIA (sPLA2-IIA), that bind to the classical RGD-binding site (site 1) of integrin Δvβ3 (8,28). Notably, fractalkine and sPLA2-IIAs activated integrins Δvβ3, α4β1, and α5β1 by binding to an additional ligand-binding site (site 2) in an allosteric manner in the absence of their receptors (e.g., CX3CR1 for fractalkine) (29, 30). The peptide from site 2 of β3 directly bound to fractalkine and sPLA2-IIA, and suppressed integrin activation by fractalkine and sPLA2-IIA, demonstrating that they directly bind to site 2 and mediate integrin activation (29,30). We recently found that stromal cell derived factor-1 (SDF1) allosterically activates these integrins by binding to site 2 (31). These findings indicate that allosteric activation of integrins by binding to site 2 is also be a common mechanism of integrin activation.

Our preliminary studies found that CD40L allosterically activated integrin Δvβ3 by binding to the allosteric binding site (site 2). This will lead to elucidation of a new signaling mechanism of CD40L/CD40/integrin signaling. Notably, CD40L binds to site 1 (the classical RGD-binding site) and site 2 (allosteric site) in a different manner. Thus, we expect that CD40L mutants that specifically block site 1 binding (e.g., Y170E) and site 2 binding (e.g., K143E) are excellent tools for studying the properties of site 2, and CD40L signaling. This is distinct from our previous studies, which found that fractalkine and sPLA2-IIA bind to site 1 and site 2 in a similar manner. For example, the same mutations of fractalkine suppress fractalkine binding to both site 1 and site 2.

Integrin binding to CD40L and genetic diseases HIGMS1 (our preliminary studies). In our preliminary studies, we located integrin-binding sites of CD40L using docking simulations and mutagenesis. Amino acid residues of CD40L that are involved in integrin binding overlap with those that have been reported to be mutated in HIGMS1 patients, indicating that the loss of integrin binding is related to HIGMS1. Consistently, mutating the amino acid residues at the integrin-binding interface of CD40L suppressed integrin binding and signaling functions of CD40L. Interestingly, the amino acid residues involved in site 2 binding are distinct from those for site 1 binding and reported to be mutated in HIGMS1 patients. Therefore, HIGMS1 (defective CD40L signaling) may be induced by the loss of integrin binding to CD40L (in addition to the effect of mutations on the trimerization and protein structure).

Dominant-negative CD40L mutants as potential therapeutics (our preliminary studies). The observation that α5β1 and CD40 can simultaneously bind to CD40L (27) is consistent with the model that they form a ternary complex. Our previous studies indicate that ternary complex (integrin-CD40L-CD40) formation on the cell surface and the integrin binding to CD40L is critical for CD40/CD40L signaling, and that the CD40L mutants defective in integrin binding are defective in signaling and dominant-negative antagonists. In our preliminary studies, we discovered that the CD40L (monomeric) mutants defective in integrin binding act as antagonists of CD40L signaling (dominant-negative). This indicates that the CD40L mutants are useful as therapeutics for chronic inflammation. The proposed project will enhance our understanding of the role of integrins in CD40L/CD40 signaling and identify novel therapeutic targets. The dominant-negative effects by the CD40L mutants are studied using reporter assays and in vivo inflammation animal models (subaim #3). We will also study how integrins contribute CD40L signaling (subaim #2).

### Preliminary Studies

The purpose of these preliminary studies was to determine if CD40L/CD40 signaling involves ternary complex formation (see significance), as we observed for several other growth factors, and if mutants defective in integrin binding are dominant-negative. Unexpectedly, we found that the loss of integrin binding to CD40L is related to HIGMS1.

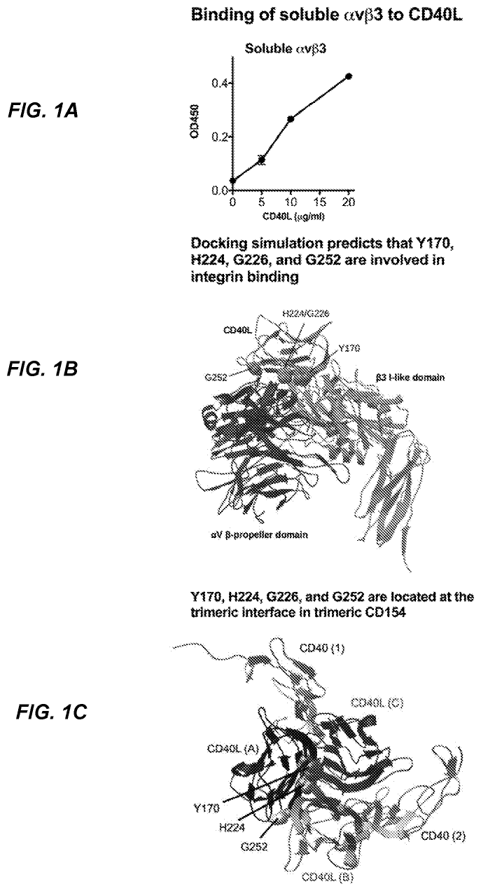

Integrin αvβ3 binds to CD40L in a KGD-independent manner. It has been reported that integrin αIIbβ3 recognizes the N-terminal KGD motif of CD40L (residues 115-117) (32). Also, integrin α5β1 binds to CD40L and the N151A/Q166A mutations on the surface of trimeric CD40L are involved in α5β1 binding (33). We studied if CD40L binds to integrin Δvβ3 using sCD40L (residues 118-262) that has no KGD motif at the N-terminus. We immobilized WT CD40L (with no KGD, Δ115-117) to wells of 96-well microtiter plates and incubated with soluble Δvβ3 in Tyrode/Hepes buffer (+1 mM MnCl₂ to fully activate Δvβ3) for 1 h, and bound Δvβ3 was quantified using anti-β3 antibody (AV10). We found that soluble Δvβ3 bound to immobilized sCD40L in ELISA-type binding assays in a dose-dependent manner (FIG. 1A). This indicates that integrin Δvβ3 is a new CD40L receptor that does not require the KGD motif for binding to sCD40L.

The integrin-binding site is located in the trimerization interface. We studied how CD40L binds to integrin Δvβ3 using docking simulation between monomeric CD40L that lacks the KGD motif (PDB code 1ALY) and the headpiece of Δvβ3 (PDB code 1L5G), which has an open headpiece conformation. Twenty out of 100 dockings grouped in the first cluster (docking energy −24.5 kcal/mol), (FIG. 1B). The simulation predicted that monomeric CD40L binds to the RGD-binding site of Δvβ3 (designated site 1). The simulation predicted that the integrin-binding site in CD40L is located in the trimerization interface (FIG. 1C). It has recently been reported that α5β1 does not require the KGD motif and that the combined N151A/Q166A in CD40L reduces α5β1 binding (33). N151 and Q166 are not located in the predicted integrin-binding site in our preliminary studies. We found that α5β1 and Δvβ3 bind to CD40L are both located in the trimeric interface of CD40L, not on the trimer surface exposed to solvent (see below).

Based on the prediction we selected several amino acid residues within the predicted integrin-binding site for mutagenesis (Tyr170, His224/Gly226, and Gly252). Notably, Tyr170 and His224, Gly226 have been reported to be mutated in HIGM1, a family of genetic disorders in which the level of IgM is relatively high as a result of a defect in CD40L signaling.

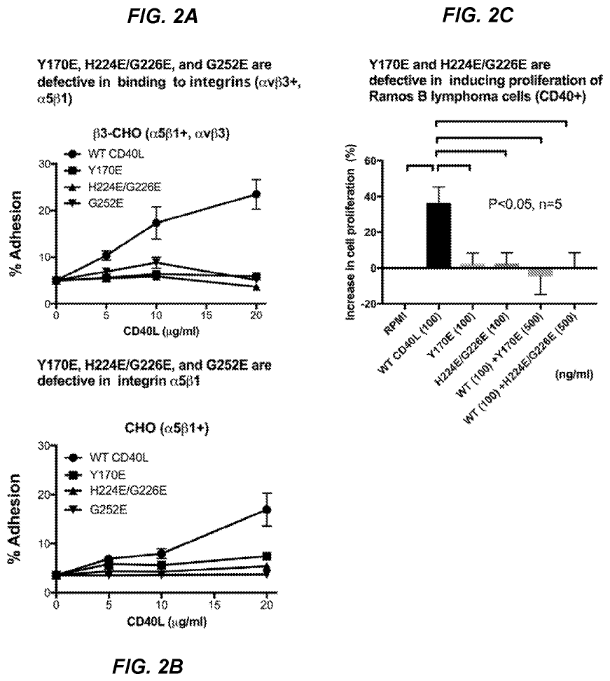

We studied the ability of the Y170 to E (Y170E), and H224E/G226E mutants to bind to Δvβ3 using CHO cells (CD40-negative) that express recombinant Δvβ3 (β3-CHO cells). Wells of 96-well microtiter plates were coated with WT and mutant CD40L and remaining protein binding sites were blocked with BSA. CHO cells and β3-CHO cells were added to the wells in DMEM and incubated for 1 h at 37° C. Bound cells were quantified using endogenous phosphatase activity (FIGS. 2A and 2B). We found that the Y170E, H224E/G226E, and G252E mutations significantly reduced binding to both Δvβ3 and α5β1 in adhesion assays. These results are consistent with the docking model. These findings demonstrate that the binding site for Δvβ3 and α5β1 is cryptic in CD40L trimer and exposed in the monomeric form. Our results indicate that integrin binding of CD40L is defective in patients in which these amino acid residues in the trimeric interface are mutated.

Also, CD40L supports adhesion of CHO cells that express recombinant Δvβ3 (β3-CHO cells, CD40-, α5β1-negative, Δvβ3-positive) better than CHO cells (α5β1-positive, Δvβ3-negative). These findings led to our conclusion that Δvβ3 is a new CD40L receptor. Our results thus indicate that monomeric CD40L, not trimeric CD40L, binds to integrins Δvβ3 and α5β1. Our docking model predicted that the one or two CD40 molecules bind to the monomeric CD40L/integrin complex, resulting in the CD40/CD40L monomer/integrin ternary complex. This is consistent with the previous report that α5β1, and CD40 can simultaneously interact with CD40L (27). It has been reported that CD40L levels markedly increase in certain pathologic conditions (e.g., SLE), and CD40L exists mainly in a monomeric form (1). It has been believed that trimeric CD40L is biologically active, but monomeric CD40L is not (34). Therefore, the role of monomeric CD40L in the pathogenesis of inflammatory diseases has been ignored.

We understand that CD40L induces ternary complex formation (integrin-CD40L-CD40) on the cell surface, and integrins and CD40 work together in CD40L, as in several other growth factors (see Significance). If ternary complex formation is critical for CD40L signaling, CD40L mutants defective in integrin binding are expected to be defective in signaling and have dominant-negative function. We studied if WT and mutant sCD40L affect proliferation of human B-cells or B-cell leukemia (FIG. 2C). Ramos cells were incubated with WT CD40L (100 ng/ml) or mutant CD40L for 48 h in RPMI (serum-free) and cell viability was determined by MTT assays. In competition assays WT CD40L (100 ng/ml) and mutant CD40L (500 ng/ml) were added. We found that WT sCD40L enhanced proliferation, but mutant CD40L did not. Furthermore, mutant sCD40L reduced proliferation enhanced by WT sCD40L (dominant-negative).

sCD40L allosterically activates integrins by binding to site 2 in an allosteric manner. It is generally understood that integrins are solely activated by inside-out signaling. We recently discovered that integrins can be activated by their own ligands in an allosteric mechanism. We recently identified that fractalkine and sPLA2-IIAs bind to the classical RGD-binding site (site 1), (8,28). Notably, fractalkine and sPLA2-IIAs activated integrins by binding to an additional ligand-binding site (site 2) in an allosteric manner (29,30).

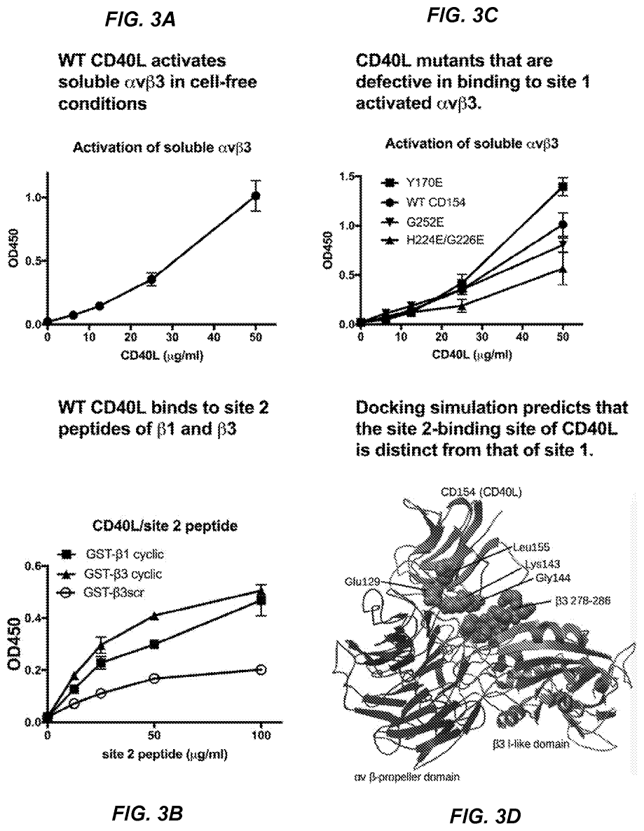

We studied if sCD40L enhances the binding of soluble Δvβ3 to its specific ligand γC399tr (binds site 1) that is immobilized in cell-free conditions (FIG. 3A). Wells of 96-well microtiter plates were coated with γC399tr (50 μg/ml in PBS) for 2 hrs at room temperature and incubated with soluble Δvβ3 (5 μg/ml) and sCD40L for 1 hour in Tyrode/HEPES with 1 mM Ca²⁺ (which keeps Δvβ3 in an inactive form). Bound Δvβ3 was quantified using anti-β3 antibody (AV10). This demonstrates that sCD40L activates Δvβ3 in an allosteric manner.

We previously reported that the peptide from site 2 of β3 (fused to GST) directly bound to fractalkine and sPLA2-IIA and suppressed integrin activation by fractalkine and sPLA2-IIA, suggesting that they directly bind to site 2 and mediate integrin activation (29, 30). We studied if CD40L binds to site 2 peptides in ELISA-type binding assays. Wells of a 96-well microtiter-plate were coated with CD40L and incubated with site 2 peptide fused to GST in PBS/0.05% Tween20 for 1 hr. Bound GST was measured using anti-GST (FIG. 3B). We found that site 2 peptide bound to sCD40L, suggesting that sCD40L binds to site 2.

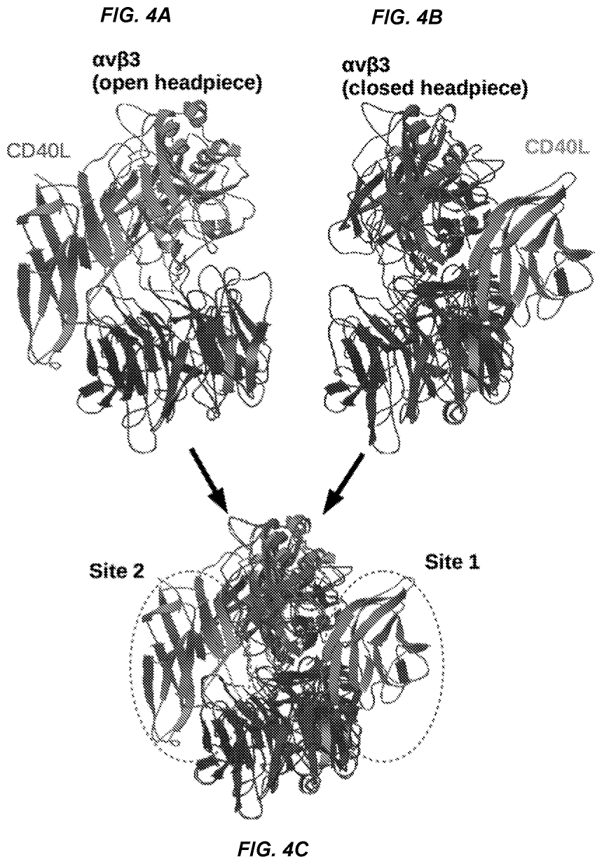

We found that Y170E, H224E/G226E, and G252E CD40L mutants, which do not bind to site 1 well (FIG. 2), activated Δvβ3 (FIG. 3C), demonstrating that CD40L binds to site 1 and site 2 in a different manner. To predict how CD40L binds to site 2, we performed docking simulations of the interaction between CD40L (1ALY.pdb) and the Δvβ3 headpiece (1JV2.pdb, closed-headpiece), (FIG. 3D). The simulation predicted that monomeric CD40L binds to site 2 well (docking energy −20.5 kcal/mol). The site 2-binding interface was predicted to be distinct from that of site 1. The predicted site 2-binding interface of CD40L included Glu129, Lys143, Glyl44, and Leu155, which have been reported to be mutated in HIGMS1. These amino acid residues are mutated to suppress binding of CD40L to site 2, and as a result suppress allosteric activation of integrins. Site 2 is located on the opposite side of site 1 in the headpiece of integrins (FIG. 4). Allosteric activation is a newly discovered mechanism of integrin activation. It has been previously believed that integrins are activated primarily by inside-out signaling. Although we have used relatively high concentrations of sCD40L to activate soluble integrins, since CD40L is expressed as a transmembrane protein on the cell surface, biological concentrations of transmembrane form CD40L should be able to activate integrins. Since CD40L binds to site 1 and site 2 differently, we will have CD40L mutants that bind only to site 2 (e.g., Y170E) and those that probably bind only to site 1 (e.g., K143T). These tools are quite useful to analyze the binding kinetics and biological role of site 2 in future studies. It is also likely that mutations in the site 2-binding interface of CD40L induce biological CD40L functional defects since some mutations of several amino acid residues in the site 2-binding interface (e.g., Glu129, Lys143, Glyl44, Leu155) are involved in HIGMS1.

### Novelty

1) We identified integrin Δvβ3 as a new CD40L receptor. 2) We discovered that the binding site of CD40L for Δvβ3 and α5β1 is located in the trimeric interface (not exposed to the surface in trimeric CD40L). This is consistent with monomeric CD40L binding to Δvβ3 and α5β1 and thereby being biologically active. We will explore how monomeric CD40L, which is elevated in serum of SLE patients, is related to inflammation. 3) We discovered that the CD40L mutants that are defective in binding to Δvβ3 and α5β1 are also defective in signaling and antagonistic. 4) We discovered that CD40L allosterically activates soluble integrin Δvβ3 in solution by binding to site 2 (allosteric binding site). 5) Many amino acid residues of CD40L involved in integrin binding are mutated in HIGMS1. Thus the loss of integrin binding and resulting loss of CD40L signaling is biologically relevant and likely related to defective immunoglobulin class switching in HIGMS1.

### Approach

### 1. Study the Role of Integrins in CD40L Signaling

A) Study how HIGMS1 and the loss of integrin binding are related. The goal of this subaim is to establish if the loss of integrin binding to CD40L is related to HIGMS1 (due to loss of CD40L signaling). Many CD40L mutations, if not all, in HIGMS are located in the trimerization interface. Thus the loss of integrin binding to CD40L is likely related to HIGMS1. To explore this relationship, we test the ability of all known HIGMS1 mutants to bind to integrins. Our preliminary studies found that several HIGMS1 mutations disrupt integrin binding to CD40L and have dominant-negative properties. Also, several amino acid residues that are involved in binding of CD40L to site 2 appear to be involved in HIGMS1 (see below). We have thus far tested three amino acid residues involved in HIGMS (Y170, H224, G226). We study if several other HIGMS1 mutants (Q174, T176, A208) are defective in integrin binding and dominant-negative. This identifies a mechanism of the genetic defects. We also introduce individual HIGMS mutations in the trimeric interface to CD40L, synthesize mutant proteins, and determine the ability of the mutants to bind to integrins in adhesion assays and binding ELISA-type assays as in our preliminary studies. In addition we determine the signaling functions of the mutants using the NF-kB reporter system and confirm that the mutants still bind to CD40.

CD40/NF-kB reporter assays: We stably express CD40 and NF-kB reporter (luciferase) in HEK293 cells and measure luciferase activity after CD40L stimulation. We include an IL-1R antagonist and anti-TNF-α to block NF-kB activation by IL-1β or TNF-α. (This HEK293 reporter system is also commercially available). To avoid NF-kB activation by IL-1β or TNF-α we include an IL-1 receptor antagonist (ILRN) and antibody to TNF-α in cell culture. We have effectively used NF-kB activation by IL-1β in recent studies (15).

CD40 binding assays: We coat wells of 96-well microtiter plates with CD40L (WT and mutants) and incubate with soluble CD40 fused to Fc (available in our lab, also commercially available). Bound CD40 is measured using anti-Fc antibodies.

Most, if not all, mutants in the integrin-binding interface and in HIGMS1 are defective in integrin binding. This is consistent with HIGMS1 and the loss of integrin binding and subsequent defective class switching from IgM to IgG being related. The HIGMS1 mutations are understood to dissociate active CD40L trimer to non-functional monomer, but it is also possible that monomeric CD40L is functional. This is explored as described below. If this is the case, the effect of HIGMS1 mutations is primarily on integrin binding. More importantly, we will establish if both CD40 and integrins are required for CD40L signaling. This verifies our biochemical evidence that CD40L induces ternary complex formation (CD40-CD40L-integrin Δvβ3).

B) Study how monomeric or trimeric CD40L induces CD40L/CD40 signaling. We previously proposed that ternary complex formation (integrin-growth factor-cognate growth factor receptor) is critically involved in growth factor signaling (ternary complex model). Growth factor mutants in which the integrin-binding site was mutated are defective in signaling and act as antagonists (see Significance). It has been generally believed that trimeric CD40L is the only active form. We found that the integrin-binding site (at least to site 1) was cryptic in trimeric CD40L. Thus, if trimeric CD40L is active, integrin binding is not involved in CD40L signaling. If integrin binding to CD40L is required, monomeric CD40L should be active and trimeric CD40L is not (since integrin-binding site is cryptic). One possibility is that active CD40L resides in a small fraction of monomeric CD40L that is dissociated from trimer. Consistent with this, very high concentrations (>1 μg/ml) of soluble CD40L are required to detect signaling in published studies. To determine if trimeric CD40L is active or not, we designed locked trimeric CD40L, in which individual CD40L monomers are disulfide-linked, to prevent dissociation of trimer. We introduced two Cys residues into individual monomers of a trimeric CD40L structure without disturbing the original conformation using the Disulfide by Design2 (DbD2) computer program (35). We selected Ala208/Ser222, Thr211/Ser213, and Ala209/Gln220 combinations for mutating to Cys. The A208C/S222C, T211C/S213C, and A209C/Q220C full-length CD40L mutants will be transfected to CHO cells (CD40L-negative). We expect that locked trimeric CD40L will be expressed on the cell surface. We check if CD40L mutants are expressed by western blotting of cell lysates in reduced and non-reduced conditions. Locked trimer has a larger size (3×) in non-reduced conditions than in reduced conditions. Seventy-two hours after transfection, cells are fixed with paraformaldehyde and used to stimulate Ramos B-cell leukemia (CD40-positive) as describe (36). We use WT full-length CD40L as a positive control, and vector only as a negative control. We select three combinations of Cys substitutions to increase likelihood that a mutant has the desired configuration.

These experiments determine if transmembrane locked trimeric CD40L is active or not. If CD40L signaling requires ternary complex (integrin-monomeric CD40L-CD40), locked trimeric CD40L is defective in signaling function (since integrin-binding site is unavailable in trimer) and show dominant-negative function. We also directly test if monomeric CD40L is active. It may be challenging to keep CD40L monomeric by introducing mutations in the trimeric interface of CD40L, since the mutation are likely to affect integrin binding in addition to trimerization. We may be able to pick the position of mutations, which block trimerization but do not affect integrin binding, and will also determine multimeric state on the cell surface when bound to CD40 by chemical crosslinking.

C) Study how leukocyte integrin α4β1 interacts with CD40L. Antibody to integrin α4 reduces relapse and progression of chronic inflammatory diseases. indicating that leukocyte integrins are involved in this signaling pathway. We examine how sCD40L binds to integrin α4β1 and activates it allosterically.

i) Binding to the classical RGD-binding site (site 1). We first study how recombinant soluble α4β1 (commercially available from R&D Systems) binds to immobilized ligands in the presence of sCD40L in the presence of Mn²⁺ (to fully activate integrins) in ELISA-type assays (see our preliminary studies). We then study how CD40L binds to these integrins on the surface of CHO cells expressing recombinant α4β1 (e.g., α4-CHO or α4-B2 variant (α5-deficient) CHO cells, available in our lab). For ligand binding assays, we label the ligands with FITC (e.g., FITC-H120, which is an α4β1-specific fibronectin ligand) and measure the bound FITC to cells in flow cytometry as described (37). Alternatively, we measure the binding of transfected cells to immobilized H120 (adhesion assays). These studies establish how CD40L binds to integrins on the cell surface.

ii) Allosteric activation of integrins: Since site 2 peptide binds to CD40L, it is understood that CD40L binds to site 2, based upon our prior studies with other chemokines/growth factors. We study how sCD40L enhances the binding of fluorescent-labeled specific ligands to integrins on the cell surface (fibronectin fragment H120 for α4β1, available in our lab), and bound FITC is quantified in flow cytometry (37). To keep the integrins inactive, we include 1 mM Ca′ in the assay medium. We also study if site 2 peptide blocks integrin activation by CD40L. Alternatively, we immobilize H120 to wells and measure the binding of U937 monocytic cells (α4+) to immobilized H120 in the presence of high [Ca²⁺].

We understand that integrin α4β1 is involved in CD40L signaling through binding to site 1 and is activated by CD40L in an allosteric mechanism through site 2. This will facilitate a detailed study of the role of integrins in B cell-T cell interactions.

Summary: This specific aim defines the integrin species that are involved in CD40L signaling and how they interact with CD40L. Importantly, we determine how the loss of integrin binding is related to HIGMS1. Further studies extend how integrin crosstalk influences CD40L regulation of immunoglobulin class switching in B cells

### 2. Determine the Role of Integrin Outside-In Signaling in CD40L Signaling

A) Study how CD40L induces integrin-CD40L-CD40 ternary complex formation. Integrin α5β1 and CD40 can simultaneously bind to CD40L in competition assays (27), but it has not been biochemically tested if CD40L induces integrin-CD40L-CD40 ternary complex. We study how CD40L induces co-precipitation of integrins (α5β1 or Δvβ3) with CD40.

Biochemical detection of the ternary complex. We incubate B cell lymphoma cell (e.g., Ramos cells) with WT CD40L (His-tagged) and purify integrins or CD40 from cell lysates by immune precipitation or pulled down using Ni-NTA Sepharose. Purified materials are analyzed by western blotting using specific antibodies to CD40 or integrins. CD40 and integrins are co-purified. Then CD40L mutant defective in integrin binding is used (see our preliminary studies). The CD40L mutants will not associate with integrins. Alternatively, we use purified soluble integrin (e.g., Δvβ3 or α5β1) and soluble CD40 (available in our lab). These proteins are mixed and incubated with WT or mutant CD40L and subjected to immunoprecipitation using anti-integrin or anti-CD40 antibodies or to pull-down using Ni-NTA Sepharose. The integrin-CD40L-CD40 ternary complex includes signaling components downstream of the complex that are involved in CD40L/CD40 signaling.

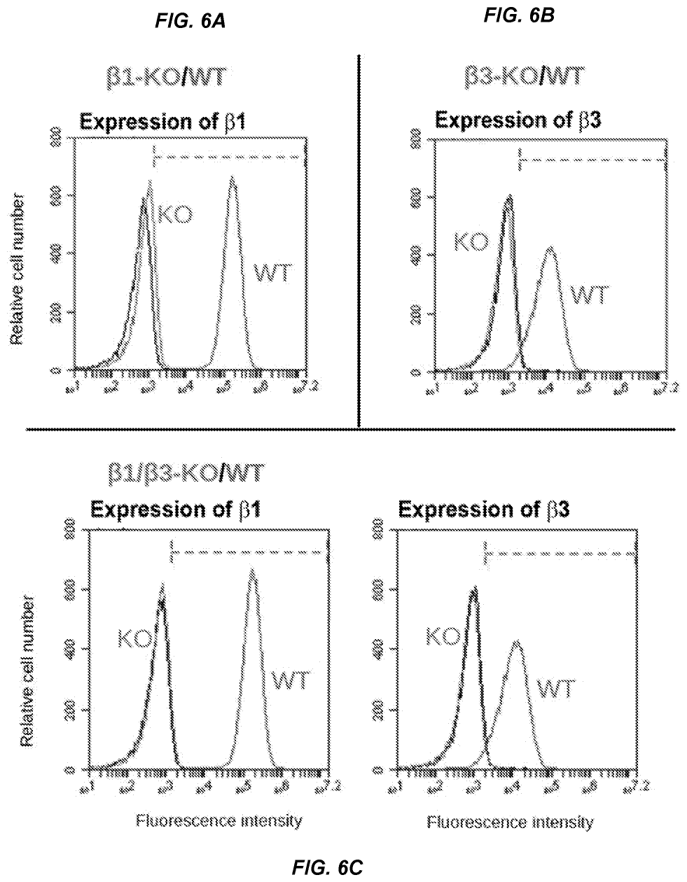

B) Study the role of the integrin outside-in signaling in CD40L signaling. Preliminary studies: Integrins β3 and/or β1 were knocked out using CRISPR/Cas9 to study the roles of β3 and β1 in HEK293 cells. Seventy-two hours after transfection of CRISPR/Cas9 constructs for integrin β3 subunit, GFP-positive cells were sorted using FACS. After 10 more days, the integrin-KO cells were detected using anti-β3. The integrin-negative/GFP-negative cells were further sorted. Because our protocol uses only transient expression of the CRISPR/Cas9 vector, Cas9 is not incorporated into the genome. Thus, we repeated the same process to knockout β1. The expression of β1 and several other proteins were not affected by β3-KO (FIG. 6). We will use the β1 and/or β3 KO cells to express mutant β1 and β3 integrins.

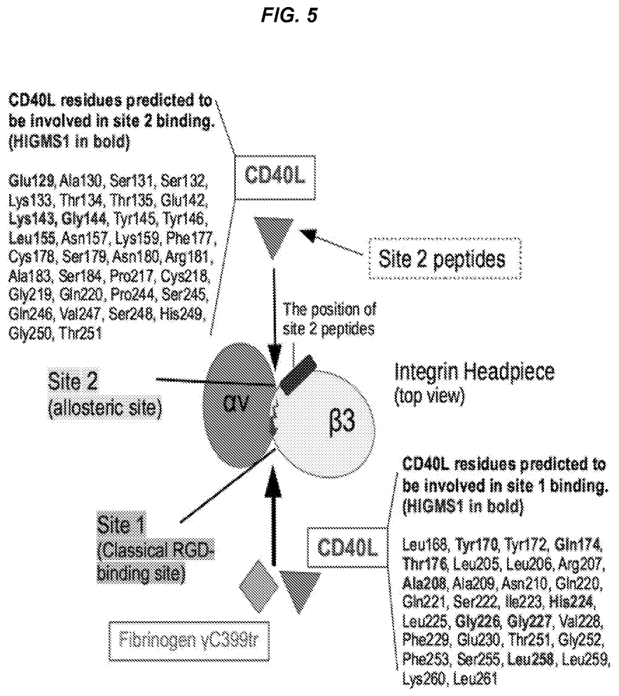

Research design: These studies enhance the understanding of how CD40L binding to integrins Δvβ3 and α5β1 induces signals that are common to other known integrin ligands (e.g., fibrinogen and fibronectin). It has recently been reported that G protein α13 (GNA13) directly binds to the integrin β3 cytoplasmic domain upon ligand binding, and mediates “outside-in signaling” from extracellular matrix (ECM) ligand fibrinogen in integrin αIIbβ3 (38). Interestingly, amino acid residues 731-733 (EAE) of the β3 cytoplasmic domain are conserved in several integrin β subunits and are critically involved in GNA13 binding to β3. Mutating 731-733 EAE to AAA (the 731-733AAA mutant) suppresses GNA13 binding (39). We first study how the β3 AAA mutation in Δvβ3 affects CD40L signaling in HEK293 cells in which human β1 and/or β3 are knocked out using CRISPR/Cas9 system (FIG. 5). We then express human CD40 and NF-kB reporter gene (luciferase). We also study how CD40L activates NF-kB in CD40/NF-kB HEK293 cells in response to CD40L as described above (40). We measure cell proliferation (e.g., MTS assays and BrdU incorporation) as described (2). These experiments establish the role of the EEE motif (and therefore GNA13 binding) in CD40L signaling. Finding that the β3 the 731-733AAA or corresponding β1 mutant is defective in mediating CD40L signaling would indicate that the outside-in signaling pathway from integrin ligands, including GNA13-binding to the β cytoplasmic domain, plays a role in CD40L signaling as well.

Following confirmation that GNA13 is involved downstream of integrins in CD40L signaling, we next study what signaling molecules are located downstream of GNA13.

If necessary, we study if any other cytoplasmic proteins bind to the β3 cytoplasmic tail (see below).

Regions of β3 cytoplasmic domain other than the EAE motif (see above) may be involved in CD40L signaling. We identify such regions using truncation mutants of the β3 tail, and subsequently identify adaptor proteins involved in CD40L signaling. In the current model, integrin-ligand binding induces conformational changes in integrins and a and β cytoplasmic tails are separated as a result (outside-in signaling). Mapping regions in the β3 cytoplasmic tail that are involved in CD40L signaling provides useful information on the proteins involved in CD40L signaling, since at least 42 “adaptor proteins” have been reported to bind to the β3 cytoplasmic tail (41). The first “hot-spot” is a membrane proximal HDRK/HDRR motif that binds to Src-family kinase Fyn, FAK (focal adhesion kinase), paxillin, and skelmin. The second and third hot-spots are membrane-proximal NPxY motif and a membrane-distal NxxY motif. Both motifs are recognition sites for phosphotyrosine-binding (PTB) domains and almost all the adaptors that bind to these motifs do so via PTB domains. Therefore, mutation/truncation of the β3 cytoplasmic tail of these hot-spots is useful for identifying the integrin-binding proteins that are involved in CD40L signaling. We have generated truncation mutants of the β3 cytoplasmic domain by introducing stop codons at positions 718, 725, 729, 737, 760, and 761 to remove hot spots. β3 mutants are transiently expressed in β3-knockout HEK293 cells in a mammalian expression vector. β3 mutants are in the form Δvβ3 (hamster αv/human β3 hybrid, this integrin is functional) together with human CD40. The ability of β3 mutants to mediate CD40L signaling is determined as described above. We determine the levels of β3 expression (median fluorescent intensity or % positive cells) in flow cytometry, and CD40L signaling using NF-kB reporter. Once the region of β3 tail that is involved in CD40L signaling is identified, we mutate individual amino acids in the region of the human β3 cytoplasmic tail (hot-spots) that are critical for CD40L signaling (that is identified by experiments using truncation mutants). These mutations include Tyr or Thr/Ser phosphorylation sites. We measure the effect of individual mutations as described above. We use dn-sCD40L and antagonists to Δvβ3 as negative controls to make sure that Δvβ3-CD40L interaction is involved.

C) Characterize CD40L-induced allosteric activation of integrins. Fractalkine and sPLA2-IIAs not only bind to the classical RGD-binding site (site 1) of integrin Δvβ3 (8, 28), but also to activated integrins Δvβ3, α4β1, and α5β1 by binding to an additional ligand-binding site (site 2) in an allosteric manner (29, 30). The peptide from site 2 of β3 directly bound to these chemokines and suppressed integrin activation by them, indicating that they directly bind to site 2 and mediate integrin activation (29, 30). Also, stromal cell derived factor-1 (SDF1) allosterically activates these integrins by binding to site 2 (31). These findings indicate that allosteric activation of integrins by binding to site 2 is a common mechanism of integrin activation. Our preliminary studies found that sCD40L enhanced the binding of soluble Δvβ3 to its specific ligand γC399tr that is immobilized in cell-free conditions in a dose-dependent manner. Also, monomeric CD40L binds strongly to site 2 in docking simulations (docking energy −20.5 kcal/mol). This indicates that sCD40L activates Δvβ3 in an allosteric manner. Interestingly, with respect to CD40L, amino acid residues of CD40L that are involved in site 1 binding were not involved in site 2 binding (FIG. 3). Consistently, CD40L mutations that suppressed the CD40L binding to site 1 (e.g., Y170E, FIG. 3) did not block integrin activation, confirming that these amino acids are NOT involved in site 2 binding. The simulation predicted that Glu129, Lys143, Glyl44, and Leu155 are involved in site 2 binding. These amino acid residues are mutated in HIGMS1 (CD40L signaling is defective). Therefore, we examine how the binding of CD40L to site 2 is involved in CD40L signaling functions. We study how CD40L mutations in the predicted site 2 binding site (e.g., Glu129, Lys143, Glyl44, Leu155) affect CD40L signaling and/or integrin activation using Ramos cell proliferation and HEK293-NF-kB reporter assays. The model is that the CD40L binding to site 2 is also involved in CD40L signaling. Also, CD40L mutants are useful tools to study the role of site 2 in detail, since CD40L mutants distinguish between two integrin binding sites (site 1 vs site 2). We measure binding kinetics of CD40L to site 1 or site 2 using mutants that bind to site 1 or site 2 specifically.

### 3. Development of Dominant-Negative CD40L Mutants as Therapeutics

In these experiments, both male and female mice are studied. In our preliminary studies, we found the integrin binding site in CD40L within the trimeric interface (e.g., Y170 and H224 for site 1), and the predicted integrin-binding site for site 2 (e.g., K143T) overlap with the amino acid residues involved in HIGMS1. Therefore, CD40L mutants defective in integrin binding are also defective in signaling, since they are involved in HIGMS1. The integrin-binding site and the CD40-binding site are separate and distinct on CD40L. Thus, most of the CD40L mutants defective in integrin binding likely still bind to CD40. In our ternary complex model of growth factor signaling (e.g., FGF, see Significance), FGF mutants defective in integrin-binding (defective in signaling function) still bind to cognate receptor (FGF receptor), and thereby compete with WT FGF for receptor binding (dominant-negative effect). The previous SAs described above establish that CD40L mutants defective in integrin binding are dominant-negative. Several CD40L mutants defective in integrin binding (e.g., Y170E) already show dominant-negative effect in cell proliferation assays (FIG. 2). More candidate CD40L mutants that are dominant negative are obtained in the proposed studies (e.g., HIGMS1 mutants and mutations that affect site 2 binding). We study the extent of their antagonistic activities using reporter assays and signaling assays (A) and in an in vivo inflammation model (B).

A) Study mechanistically how CD40L mutants defective in integrin binding are dominant-negative (dnCD40L). Antigen presenting cells (APCs), including dendritic cells (DCs), macrophages and B cells, are T cell priming factors in autoimmune diseases. CD40 is constitutively expressed by all antigen presenting cells (APCs), including dendritic cells, macrophages and B cells. CD40L is expressed by T cells. CD40-CD40L interactions are critical for effector T cell activation. Since development of antibodies to CD40L has been halted due to thromboembolism events in clinical trials, it is important to develop an alternative strategy to block CD40L functions. Our preliminary studies suggest that CD40L mutants defective in integrin binding (site 1) are defective in CD40L signaling, and we provide evidence that CD40L mutants defective in integrin binding are also dominant-negative (see our preliminary studies): CD40L mutants suppressed proliferation of Ramos cells induced by WT CD40L. This observation in CD40L is consistent with our previous studies, in which several growth factor mutants defective in integrin binding were dominant-negative (antagonistic), (see Significance section). Our preliminary studies indicated that CD40L mutants that are defective in integrin binding are candidates for dominant-negative antagonists of CD40L signaling. Here we further study this using the CD40/NF-kB reporter system.

Reporter assays: We use HEK293 cells that stably express CD40 and NF-kB reporter (luciferase) expression constructs as described above. When cells are stimulated with CD40L, NF-kB activation induces luciferase expression and is detected in cell lysates. CD40L mutants defective in integrin binding will be antagonistic and suppress NF-kB activation by WT CD40L. The reporter assays are more sensitive and easier to quantify, and thus allow us to determine to what degree CD40L mutants defective in integrin binding are antagonistic.

Assays of the CD40L-CD40 pathway: Cellular assays of the CD40L-CD40 pathway are performed by measuring activation of antigen-educated B cells from OT II transgenic mice (OT-II/B6) (42). These OT-II/B6 mice contain an engineered CD4 co-receptor specific for chicken ovalbumin (OVA) 323-339 peptide able to interact with MHC class II, resulting in CD4 T cells predominantly specific for this antigen. Th1 polarized T cells are prepared from splenocytes of OT-II/B6 mice incubated with 1 μM OVA 323-339 peptide and 10 μg/mL anti-IL-4; antigen-educated B cells are prepared from OT-II splenocytes pulsed with OVA peptide and 2 μM cyclosporine A for 2 h. Each cell type is purified by magnetic separation. Measure of B cell activation is determined by flow cytometry for surface expression of ICAM-1 after overnight incubation of antigen-educated B and Th1 polarized T cells. CD40 dependence on antigen-dependent cell activation will be identified with dnCD40L, or neutralizing CD40L antibody as a control.

CD40 binding assay: Our preliminary studies indicate that the integrin binding site and CD40-binding site are distinct on CD40L. If integrin binding is reduced the CD40L mutants will not show antagonistic activity. We verify that each CD40L mutation that affects integrin binding is distinct from CD40 binding and also measure binding properties of soluble recombinant CD40-Fc fusion protein to immobilized CD40L in ELISA-type assays, and dissociation constants by surface plasmon resonance.

In possible other studies, we introduce double mutations that suppress binding to both site 1 and site 2 (e.g., Y170E/K143T), and examine if the double mutation further reduces CD40L signaling.

B) Study the effect of dn-sCD40L in a mouse model of skin inflammation in psoriasis. CD40-CD40L interactions are critical for effector T cell activation, and are present on APCs and T cells, respectively. APCs, including DCs, macrophages and B cells, are understood to be T cell priming factors in skin inflammation, and these interactions may be studied in inflammatory skin models. Psoriasis is a common skin disorder mediated by crosstalk between epidermal keratinocytes (KCs), dermal vascular cells, and immune cells, including activated DCs, Th1 cells, and Th17 cells (43). The disease is characterized by epidermal hyperproliferation, leukocyte infiltration, and vascular proliferation in the papillary dermis, which leads to the clinical features of bright red scaly plaques (44). Skin inflammation in Imiquimod (IMQ)-induced psoriasis assessed by erythema, scaling, and epidermal thickness was significantly reduced in CD40(−/−) mice compared with wild-type mice, accompanied by decreases in inflammatory cytokine production (45). Thus, CD40L/CD40 signaling plays a role in this psoriasis model.

In our preliminary studies, dn-sCD40L suppressed CD40L signaling, indicating that dn-sCD40L is an antagonist of CD40L and has usefulness as a therapeutic agent for inflammation. We use an animal model of psoriasis to study the efficacy of dn-sCD40L, and assess how dn-sCD40L suppresses psoriasis. The studies described here are significant because dn-sCD40L can be used as a therapeutic agent. We include glucocorticoid-treated disease mice as controls and observe how dn-sCD40L and its derivatives suppress skin inflammations by blocking leukocyte recruitment, but not seriously compromise patients' ability to respond to infections. We test if dn-sCD40L suppresses inflammation in an Imiquimod (IMQ)-induced psoriasis model. We use several inflammatory parameters including clinical scores, cellular infiltration, and cytokine production, and determine the optimum doses for dn-sCD40L. We also determine suitable routes of administration of dn-sCD40L, e.g., at the site of inflammation, or systemically.

Psoriasis-like model: Imiquimod (IMQ), an agonist of toll like receptor (TLR) 7/8, activates immune cells, such as macrophages and plasmacytoid DCs (46). Daily application of IMQ on mouse skin triggers Th1 and Th17 cell-mediated adaptive immunity and mice rapidly exhibit thickened skin with erythema and scales resembling psoriasis (47). It has been reported that skin inflammation was significantly reduced in CD40-null mice compared with WT mice, which was accompanied by decreases in inflammatory cytokine production, demonstrating that this model is useful for evaluating the effect of our dnCD40L. Mice (BALB/c or C57BL/6) at 8 to 10 weeks of age are topically challenged with 62.5 mg of commercially available IMQ cream (5% Aldara; 3M Pharmaceuticals) or control vehicle cream (Vaseline) on the shaved back and the right ear for 7 consecutive days. dn-sCD40L is locally administrated on the first day of IMQ challenge. The ear thickness is measured before the daily challenge. After a weeks' challenge, mice are euthanized and ears and back skin will be collected for hematoxylin and eosin stain (H&E), immunohistochemistry (LHC) and flow cytometry. As an alternative, we test the effects of CD40L mutant on the IL-23 induced psoriasis model. Intradermal injection of carrier-free recombinant IL-23 protein (1 μg) into the right ear of anesthetized mice is performed using an insulin-syringe every other day for 14 days. The effects on skin thickness, skin cell infiltration and cytokine levels are evaluated as described earlier.

Local administration of dn-sCD40L using gelatin hydrogel for sustained release in mouse models: We use both male and female mice. Biodegradable gelatin hydrogel has been widely used to enhance the in vivo effect of cytokines (WT FGF2), (e.g., angiogenesis, bone formation, and wound healing (48-52)). Gelatin is a denatured collagen commercially available as a bioabsorbable polymer. Two types of gelatin have been prepared by different processes from collagen to yield two products with isoelectric points (IEPs) of 5.0 and 9.0 (acidic gelatin and basic gelatin, respectively). Acidic gelatin hydrogel ionically adsorbs cytokines with high IEP (>IEP 8.5. e.g., FGF2), which are then slowly released over 2 weeks in vivo. Once ionically absorbed cytokines are protected from degradation and not released until gelatin hydrogel is proteolytically degraded. Thus, gelatin hydrogel effectively enhances the in vivo effect of cytokines (e.g., angiogenesis and bone formation by FGF2). We mix dn-sCD40L and acidic gelatin hydrogel (low endotoxin, molecular weight 100 kda, commercially available) at molar ratio 1:1 and incubate at 37° C. for 1 hr. We have previously successfully used gelatin hydrogel to block angiogenesis in vivo by a dominant-negative mutant of FGF2 (53). The dn-sCD40L/gelatin hydrogel mixture is injected subcutaneously in the diseased tissue in mice. Gelatin hydrogel without dn-sCD40L is injected as a control. Amounts injected locally are optimized for efficiency. The local injection studies provide information about efficacy of dn-sCD40L and potential toxicity. Alternatively, dn-sCD40L/gelatin hydrogel mixture is delivered intraperitoneally for sustained systemic release. Undesired side effects of immune disruption are determined by assessing anti-microbial resistance, and antibody production.

Our dn-sCD40L is synthesized in E. coli and purified using Ni-NTA affinity chromatography and further purified by gel-filtration in FPLC. To remove endotoxin, use recently developed endotoxin-free E. coli BL21 (commercially available). dn-sCD40L is endotoxin-free and transplanted locally with gelatin hydrogel. The epidermal thickness is determined by averaging at least 10 random locations on each slide. Immunohistochemistry (IHC) of skin section is used to evaluate the infiltrated immune cells using cell type specific markers. The NF-kB levels in skin are also evaluated by IHC. We use immunoblotting of skin homogenates to detect NF-kB or p-NF-kB to evaluate inflammation as described (54). mRNA levels of cytokine (IL-23 or IL-17, IL-8) from ear or back skin are analyzed by RT-PCR. Cell types such as T cells, B cells, macrophages, and dendritic cells are analyzed from single cells collected from spleen or back skin after collagenase digestion. The collected cells are stained with dye-conjugated antibodies specific to each cell type.

Effective dn-sCD40L inhibitors are useful as novel and effective agents, and have advantages over other types of CD40 inhibitors because of higher specificity to cognate receptors over kinase inhibitors, and may have better penetrance into the diseased tissues than IgG (180K) because of their smaller sizes (<29K). Gelatin hydrogel is a useful carrier of proteins, which can release the cargo slowly for a long period (2 weeks), and is biocompatible and biodegradable. The experiments described herein establish that dn-sCD40L/gelatin hydrogel effectively suppresses disease progression.

Gene therapy using expression construct encoding dn-sCD40L: Angiogenic gene therapy of diabetic angiopathy (e.g., limb ischemia) has been performed in clinical trial (TALISMAN) using a WT FGF1 expression vector in a non-viral expression vector (55). Vector DNA was injected to the muscle tissue of the affected limb. The study revealed that the risk of major amputation was reduced in half of patients who received the expression vector over a period of 6 weeks (56). This strategy is exploited to administer dn-sCD40L and construct a mammalian expression vector with cDNA encoding dn-sCD40L. The expression vector with a luciferase gene is used to confirm that gene expression occurs in diseased tissues in vivo. The expression vector (a single injection) expresses dn-sCD40L locally.

Statistical analysis and power analysis: Levels of inflammation are compared across groups using standard repeated measures mixed models (57). These models allow for possibly unequal spacing of measurements or unequal lengths of follow-up, as, for example, if some mice develop unsustainable tumor burdens and are sacrificed early. We formulate these models to test specifically for dn-sCD40L-treated mice vs control, then test for the added impact of increasing doses, to identify an optimal dose level, on the levels of inflammation. We found that power analysis in the previous in vivo experiments is such that a 20% difference between treatment and control groups can be detected with 8 mice in each group (58, 59). We thus typically use 10-12 mice per group, unless we have pilot data that suggests a much better than 20% effect, in which case we use 8 mice per group.

These experiments assess the ability of dn-sCD40L to suppress inflammatory indicators in psoriasis, in particular suppression of leukocyte recruitment to the inflammation areas and suppression of inflammatory cytokine production but not affect humoral and cellular immune responses (including antibody production). These experiments establish CD40L-integrin interaction as a novel therapeutic target and that dn-sCD40L is useful as a therapeutic agent.

As necessary, the timing, doses, and/or delivery methods (e.g., ip or iv) for dn-sCD40L are optimized. Optionally, commercially available slow-delivery pumps are used to inject continuously. If immunogenicity of the human CD40L mutant is a problem in mice, determined by presence of antibodies to human dn-sCD40L, a mouse version of dn-sCD40L (60) is used.

Summary: The proposed project enhances our understanding of CD40L/CD40 signaling and the role of integrins in CD40L signaling as a co-receptor, and establishes how CD40L mutations that suppress integrin binding correlate with mutations responsible for HIGMS1. These studies also further the development of dominant-negative CD40L mutants and evaluation of their efficacy in an animal model of inflammation as potential therapeutic agents.

### REFERENCES

1. Alaaeddine, N., G. S. Hassan, D. Yacoub, and W. Mourad, CD154: an immunoinflammatory mediator in systemic lupus erythematosus and rheumatoid arthritis. Clin Dev Immunol, 2012. 2012: p. 490148. 10.1155/2012/490148
2. Mori, S., C. Y. Wu, S. Yamaji, J. Saegusa, B. Shi, Z. Ma, Y. Kuwabara, K. S. Lam, R. R. Isseroff, Y. K. Takada, and Y. Takada, Direct Binding of Integrin {alpha}v{beta}3 to FGF1 Plays a Role in FGF1 Signaling. J Biol Chem, 2008. 283(26): p. 18066-75. M801213200 [pii] 10.1074/jbc.M801213200
3. Saegusa, J., S. Yamaji, K. Ieguchi, C. Y. Wu, K. S. Lam, F. T. Liu, Y. K. Takada, and Y. Takada, The direct binding of insulin-like growth factor-1 (IGF-1) to integrin alphavbeta3 is involved in IGF-1 signaling. J Biol Chem, 2009. 284(36): p. 24106-14. M109.013201 [pii] 10.1074/jbc.M109.013201
4. Ieguchi, K., M. Fujita, Z. Ma, P. Davari, Y. Taniguchi, K. Sekiguchi, B. Wang, Y. K. Takada, and Y. Takada, Direct binding of the EGF-like domain of neuregulin-1 to integrins ({alpha}v{beta}3 and {alpha}6{beta}4) is involved in neuregulin-1/ErbB signaling. J Biol Chem, 2010. 285(41): p. 31388-98. M110.113878 [pii] 10.1074/jbc.M110.113878
5. Yamaji, S., J. Saegusa, K. Ieguchi, M. Fujita, Y. K. Takada, and Y. Takada, A novel fibroblast growth factor-1 (FGF1) mutant that acts as an FGF antagonist. PLoS One, 2010. 5(4): p. e10273. 10.1371/journal.pone.0010273
6. Mori, S., V. Tran, K. Nishikawa, T. Kaneda, Y. Hamada, N. Kawaguchi, M. Fujita, Y. K. Takada, N. Matsuura, M. Zhao, and Y. Takada, A Dominant-Negative FGF1 Mutant (the R50E Mutant) Suppresses Tumorigenesis and Angiogenesis. PLoS One, 2013. 8(2): p. e57927. 10.1371/journal.pone.0057927
7. Fujita, M., K. Ieguchi, D. M. Cedano-Prieto, A. Fong, C. Wilkerson, J. Q. Chen, M. Wu, S. H. Lo, A. T. Cheung, M. D. Wilson, R. D. Cardiff, A. D. Borowsky, Y. K. Takada, and Y. Takada, An Integrin Binding-defective Mutant of Insulin-like Growth Factor-1 (R36E/R37E IGF1) Acts as a Dominant-negative Antagonist of the IGF1 Receptor (IGF1R) and Suppresses Tumorigenesis but Still Binds to IGF1R. J Biol Chem, 2013. 288(27): p. 19593-603. 10.1074/jbc.M113.470872
8. Fujita, M., Y. K. Takada, and Y. Takada, Integrins alphavbeta3 and alpha4beta1 Act as Coreceptors for Fractalkine, and the Integrin-Binding Defective Mutant of Fractalkine Is an Antagonist of CX3CR1. J Immunol, 2012. 189(12): p. 5809-5819. 10.4049/jimmunol.1200889
9. Seyama, K., S. Nonoyama, I. Gangsaas, D. Hollenbaugh, H. F. Pabst, A. Aruffo, and H. D. Ochs, Mutations of the CD40 ligand gene and its effect on CD40 ligand expression in patients with X-linked hyper IgM syndrome. Blood, 1998. 92(7): p. 2421-34.
10. Jones, J. I., T. Prevette, A. Gockerman, and D. R. Clemmons, Ligand occupancy of the alpha-V-beta3 integrin is necessary for smooth muscle cells to migrate in response to insulin-like growth factor. Proc Natl Acad Sci USA, 1996. 93(6): p. 2482-7.
11. Brooks, P. C., R. A. Clark, and D. A. Cheresh, Requirement of vascular integrin alpha v beta 3 for angiogenesis. Science, 1994. 264(5158): p. 569-71.
12. Kim, S. H., J. Turnbull, and S. Guimond, Extracellular matrix and cell signalling: the dynamic cooperation of integrin, proteoglycan and growth factor receptor. J Endocrinol, 2011. 209(2): p. 139-51. 10.1530/JOE-10-0377
13. Odenthal, J., R. Takes, and P. Friedl, Plasticity of tumor cell invasion: governance by growth factors and cytokines. Carcinogenesis, 2016. 37(12): p. 1117-1128. 10.1093/carcin/bgw098
14. Desgrosellier, J. S. and D. A. Cheresh, Integrins in cancer: biological implications and therapeutic opportunities. Nat Rev Cancer, 2010. 10(1): p. 9-22. 10.1038/nrc2748
15. Takada, Y. K., J. Yu, M. Fujita, J. Saegusa, C. Y. Wu, and Y. Takada, Direct binding to integrins and loss of disulfide linkage in interleukin-1beta (IL-1beta) are involved in the agonistic action of IL-1beta. J Biol Chem, 2017. 292(49): p. 20067-20075. 10.1074/jbc.M117.818302
16. Grewal, I. S. and R. A. Flavell, CD40 and CD154 in cell-mediated immunity. Annu Rev Immunol, 1998. 16: p. 111-35. 10.1146/annurev.immunol.16.1.111
17. Chatzigeorgiou, A., M. Lyberi, G. Chatzilymperis, A. Nezos, and E. Kamper, CD40/CD40L signaling and its implication in health and disease. Biofactors, 2009. 35(6): p. 474-83. 10.1002/biof.62
18. Schonbeck, U. and P. Libby, The CD40/CD154 receptor/ligand dyad. Cell Mol Life Sci, 2001. 58(1): p. 4-43.
19. Cha, J. K., M. H. Jeong, H. R. Bae, J. Y. Han, S. J. Jeong, H. J. Jin, Y. J. Lim, S. H. Kim, and J. W. Kim, Activated platelets induce secretion of interleukin-1beta, monocyte chemotactic protein-1, and macrophage inflammatory protein-1alpha and surface expression of intercellular adhesion molecule-1 on cultured endothelial cells. J Korean Med Sci, 2000. 15(3): p. 273-8. 10.3346/jkms.2000.15.3.273
20. Daub, K., H. Langer, P. Seizer, K. Stelios, A. E. May, P. Goyal, B. Bigalke, T. Schonberger, T. Geisler, D. Siegel-Axel, R. A. Oostendorp, S. Lindemann, and M. Gawaz, Platelets induce differentiation of human CD34+ progenitor cells into foam cells and endothelial cells. FASEB J, 2006. 20(14): p. 2559-61. 10.1096/fj.06-6265fje
21. Henn, V., J. R. Slupsky, M. Grafe, I. Anagnostopoulos, R. Forster, G. Muller-Berghaus, and R. A. Kroczek, CD40 ligand on activated platelets triggers an inflammatory reaction of endothelial cells. Nature, 1998. 391(6667): p. 591-4. 10.1038/35393
22. Crow, M. K. and K. A. Kirou, Regulation of CD40 ligand expression in systemic lupus erythematosus. Curr Opin Rheumatol, 2001. 13(5): p. 361-9.
23. Delmas, Y., J. F. Viallard, A. Solanilla, J. Villeneuve, J. M. Pasquet, F. Belloc, I. Dubus, J. Dechanet-Merville, P. Merville, P. Blanco, J. L. Pellegrin, A. T. Nurden, C. Combe, and J. Ripoche, Activation of mesangial cells by platelets in systemic lupus erythematosus via a CD154-dependent induction of CD40. Kidney Int, 2005. 68(5): p. 2068-78. 10.1111/j.1523-1755.2005.00663.x
24. Hassan, G. S., J. Stagg, and W. Mourad, Role of CD154 in cancer pathogenesis and immunotherapy. Cancer Treat Rev, 2015. 41(5): p. 431-40. 10.1016/j.ctrv.2015.03.007
25. Hassan, G. S., Y. Merhi, and W. Mourad, CD40 ligand: a neo-inflammatory molecule in vascular diseases. Immunobiology, 2012. 217(5): p. 521-32. 10.1016/j.imbio.2011.03.010
26. Andre, P., K. S. Prasad, C. V. Denis, M. He, J. M. Papalia, R. O. Hynes, D. R. Phillips, and D. D. Wagner, CD40L stabilizes arterial thrombi by a beta3 integrin—dependent mechanism. Nat Med, 2002. 8(3): p. 247-52. 10.1038/nm0302-247
27. El Fakhry, Y., H. Alturaihi, D. Yacoub, L. Liu, W. Guo, C. Leveille, D. Jung, L. B. Khzam, Y. Merhi, J. A. Wilkins, H. Li, and W. Mourad, Functional interaction of CD154 protein with alpha5beta1 integrin is totally independent from its binding to alphaIIbbeta3 integrin and CD40 molecules. J Biol Chem, 2012. 287(22): p. 18055-66. 10.1074/jbc.M111.333989
28. Saegusa, J., N. Akakura, C. Y. Wu, C. Hoogland, Z. Ma, K. S. Lam, F. T. Liu, Y. K. Takada, and Y. Takada, Pro-inflammatory secretory phospholipase A2 type IIA binds to integrins alphavbeta3 and alpha4beta1 and induces proliferation of monocytic cells in an integrin-dependent manner. J Biol Chem, 2008. 283(38): p. 26107-15. M804835200 [pii] 10.1074/jbc.M804835200
29. Fujita, M., Y. K. Takada, and Y. Takada, The chemokine fractalkine can activate integrins without CX3CR1 through direct binding to a ligand-binding site distinct from the classical RGD-binding site. PLoS One, 2015. 9(5): p. e96372. 10.1371/journal.pone.0096372
30. Fujita, M., K. Zhu, C. K. Fujita, M. Zhao, K. S. Lam, M. J. Kurth, Y. K. Takada, and Y. Takada, Proinflammatory secreted phospholipase A2 type IIA (sPLA-IIA) induces integrin activation through direct binding to a newly identified binding site (site 2) in integrins alphavbeta3, alpha4beta1, and alpha5beta1. J Biol Chem, 2015. 290(1): p. 259-71. 10.1074/jbc.M114.579946
31. Fujita, M., P. Davari, Y. K. Takada, and Y. Takada, Stromal cell-derived factor-1 (CXCL12) activates integrins by direct binding to an allosteric ligand-binding site (site 2) of integrins without CXCR4. Biochem J, 2018. 10.1042/BCJ20170867
32. Singh, J., E. Garber, H. Van Vlijmen, M. Karpusas, Y. M. Hsu, Z. Zheng, J. H. Naismith, and D. Thomas, The role of polar interactions in the molecular recognition of CD40L with its receptor CD40. Protein Sci, 1998. 7(5): p. 1124-35. 10.1002/pro.5560070506
33. Alturaihi, H., G. S. Hassan, L. Al-Zoobi, S. Salti, Y. Darif, D. Yacoub, S. El Akoum, M. Oudghiri, Y. Merhi, and W. Mourad, Interaction of CD154 with different receptors and its role in bidirectional signals. Eur J Immunol, 2015. 45(2): p. 592-602. 10.1002/eji.201444941
34. Pietravalle, F., S. Lecoanet-Henchoz, J. P. Aubry, G. Elson, J. Y. Bonnefoy, and J. F. Gauchat, Cleavage of membrane-bound CD40 ligand is not required for inducing B cell proliferation and differentiation. Eur J Immunol, 1996. 26(3): p. 725-8. 10.1002/eji.1830260333
35. Craig, D. B. and A. A. Dombkowski, Disulfide by Design 2.0: a web-based tool for disulfide engineering in proteins. BMC Bioinformatics, 2013. 14: p. 346. 10.1186/1471-2105-14-346
36. Armitage, R. J., W. C. Fanslow, L. Strockbine, T. A. Sato, K. N. Clifford, B. M. Macduff, D. M. Anderson, S. D. Gimpel, T. Davis-Smith, C. R. Maliszewski, and et al., Molecular and biological characterization of a murine ligand for CD40. Nature, 1992. 357(6373): p. 80-2. 10.1038/357080a0
37. Fujita, M., Y. K. Takada, and Y. Takada, The chemokine fractalkine can activate integrins without CX3CR1 through direct binding to a ligand-binding site distinct from the classical RGD-binding site. PLoS One, 2014. 9(5): p. e96372. 10.1371/journal.pone.0096372
38. Gong, H., B. Shen, P. Flevaris, C. Chow, S. C. Lam, T. A. Voyno-Yasenetskaya, T. Kozasa, and X. Du, G protein subunit Galpha13 binds to integrin alphaIIbbeta3 and mediates integrin “outside-in” signaling. Science, 2010. 327(5963): p. 340-3. 10.1126/science.1174779
39. Shen, B., X. Zhao, K. A. O'Brien, A. Stojanovic-Terpo, M. K. Delaney, K. Kim, J. Cho, S. C. Lam, and X. Du, A directional switch of integrin signalling and a new anti-thrombotic strategy. Nature, 2013. 503(7474): p. 131-5. 10.1038/nature12613
40. Lossner, D., C. Abou-Ajram, A. Benge, and U. Reuning, Integrin alphavbeta3 mediates upregulation of epidermal growth-factor receptor expression and activity in human ovarian cancer cells. Int J Biochem Cell Biol, 2008. 40(12): p. 2746-61. 51357-2725(08)00215-X [pii] 10.1016/j.bioce1.2008.05.014
41. Legate, K. R. and R. Fassler, Mechanisms that regulate adaptor binding to beta-integrin cytoplasmic tails. Journal of cell science, 2009. 122(Pt 2): p. 187-98. 10.1242/jcs.041624
42. Gardell, J. L. and D. C. Parker, CD40L is transferred to antigen-presenting B cells during delivery of T-cell help. Eur J Immunol, 2017. 47(1): p. 41-50. 10.1002/eji.201646504
43. Golden, J. B., T. S. McCormick, and N. L. Ward, IL-17 in psoriasis: implications for therapy and cardiovascular co-morbidities. Cytokine, 2013. 62(2): p. 195-201. 10.1016/j.cyto.2013.03.013
44. Nestle, F. O., D. H. Kaplan, and J. Barker, Psoriasis. N Engl J Med, 2009. 361(5): p. 496-509. 10.1056/NEJMra0804595
45. Morimura, S., T. Oka, M. Sugaya, and S. Sato, CX3CR1 deficiency attenuates imiquimod-induced psoriasis-like skin inflammation with decreased M1 macrophages. J Dermatol Sci, 2016. 82(3): p. 175-88. 10.1016/j.jdermsci.2016.03.004
46. Kan, Y., T. Okabayashi, S. Yokota, S. Yamamoto, N. Fujii, and T. Yamashita, Imiquimod suppresses propagation of herpes simplex virus 1 by upregulation of cystatin A via the adenosine receptor A1 pathway. J Virol, 2012. 86(19): p. 10338-46. 10.1128/JVI.01196-12
47. van der Fits, L., S. Mourits, J. S. Voerman, M. Kant, L. Boon, J. D. Laman, F. Cornelissen, A. M. Mus, E. Florencia, E. P. Prens, and E. Lubberts, Imiquimod-induced psoriasis-like skin inflammation in mice is mediated via the IL-23/IL-17 axis. J Immunol, 2009. 182(9): p. 5836-45. 10.4049/jimmunol.0802999
48. Matsui, M. and Y. Tabata, Enhanced angiogenesis by multiple release of platelet-rich plasma contents and basic fibroblast growth factor from gelatin hydrogels. Acta Biomater, 2012. 8(5): p. 1792-801. 10.1016/j.actbio.2012.01.016
49. Sasaki, N., T. Minami, K. Yamada, H. Yamada, Y. Inoue, M. Kobayashi, and Y. Tabata, In vivo effects of intra-articular injection of gelatin hydrogen microspheres containing basic fibroblast growth factor on experimentally induced defects in third metacarpal bones of horses. Am J Vet Res, 2008. 69(12): p. 1555-9. 10.2460/ajvr.69.12.1555
50. Ichinohe, N., Y. Kuboki, and Y. Tabata, Bone regeneration using titanium nonwoven fabrics combined with fgf-2 release from gelatin hydrogel microspheres in rabbit skull defects. Tissue Eng Part A, 2008. 14(10): p. 1663-71. 10.1089/ten.tea.2006.0350
51. Ishimaru, T., M. Komura, M. Sugiyama, H. Komura, M. Arai, J. Fujishiro, C. Uotani, K. Miyakawa, T. Kakihara, K. Hoshi, T. Takato, Y. Tabata, H. Komuro, and T. Iwanaka, Slow release of basic fibroblast growth factor (b-FGF) enhances mechanical properties of rat trachea. J Pediatr Surg, 2015. 50(2): p. 255-9. 10.1016/j.jpedsurg.2014.11.012
52. Narita, A., M. Takahara, D. Sato, T. Ogino, S. Fukushima, Y. Kimura, and Y. Tabata, Biodegradable gelatin hydrogels incorporating fibroblast growth factor 2 promote healing of horizontal tears in rabbit meniscus. Arthroscopy, 2012. 28(2): p. 255-63. 10.1016/j.arthro.2011.08.294
53. Mori, S., N. Hatori, N. Kawaguchi, Y. Hamada, T. C. Shih, C. Y. Wu, K. S. Lam, N. Matsuura, H. Yamamoto, Y. K. Takada, and Y. Takada, The integrin-binding defective FGF2 mutants potently suppress FGF2 signalling and angiogenesis. Biosci Rep, 2017. 37(2). 10.1042/BSR20170173
54. Wu, Y., X. Chen, X. Ge, H. Xia, Y. Wang, S. Su, W. Li, T. Yang, M. Wei, H. Zhang, L. Gou, J. Li, X. Jiang, and J. Yang, Isoliquiritigenin prevents the progression of psoriasis-like symptoms by inhibiting NF-kappaB and proinflammatory cytokines. J Mol Med (Berl), 2016. 94(2): p. 195-206. 10.1007/s00109-015-1338-3
55. Nikol, S., I. Baumgartner, E. Van Belle, C. Diehm, A. Visoá, M. C. Capogrossi, N. Ferreira-Maldent, A. Gallino, M. Graham Wyatt, L. Dinesh Wijesinghe, M. Fusari, D. Stephan, J. Emmerich, G. Pompilio, F. Vermassen, E. Pham, V. Grek, M. Coleman, and F. Meyer, Therapeutic Angiogenesis With Intramuscular NV1FGF Improves Amputation-free Survival in Patients With Critical Limb Ischemia. Molecular Therapy, 2008. 16(5): p. 972-978. 10.1038/mt.2008.33
56. Ruck, A. and C. Sylven, Therapeutic angiogenesis gains a leg to stand on. Mol Ther, 2008. 16(5): p. 808-10. 10.1038/mt.2008.65
57. Laird, N. M. and J. H. Ware, Random-effects models for longitudinal data. Biometrics, 1982. 38(4): p. 963-74.
58. Namba, R., L. J. Young, C. K. Abbey, L. Kim, P. Damonte, A. D. Borowsky, J. Qi, C. G. Tepper, C. L. MacLeod, R. D. Cardiff, and J. P. Gregg, Rapamycin inhibits growth of premalignant and malignant mammary lesions in a mouse model of ductal carcinoma in situ. Clin Cancer Res, 2006. 12(8): p. 2613-21.
59. Namba, R., L. J. Young, J. E. Maglione, E. T. McGoldrick, S. Liu, G. T. Wurz, M. W. DeGregorio, A. D. Borowsky, C. L. MacLeod, R. D. Cardiff, and J. P. Gregg, Selective estrogen receptor modulators inhibit growth and progression of premalignant lesions in a mouse model of ductal carcinoma in situ. Breast Cancer Res, 2005. 7(6): p. R881-9. bcr1317 [pii] 10.1186/bcr1317
60. Pan, Y., C. Lloyd, H. Zhou, S. Dolich, J. Deeds, J. A. Gonzalo, J. Vath, M. Gosselin, J. Ma, B. Dussault, E. Woolf, G. Alperin, J. Culpepper, J. C. Gutierrez-Ramos, and D. Gearing, Neurotactin, a membrane-anchored chemokine upregulated in brain inflammation. Nature, 1997. 387(6633): p. 611-7. 10.1038/42491
61. Nanki, T., Y. Urasaki, T. Imai, M. Nishimura, K. Muramoto, T. Kubota, and N. Miyasaka, Inhibition of fractalkine ameliorates murine collagen-induced arthritis. J Immunol, 2004. 173(11): p. 7010-6.

### Key Resources

HEK293 cells in which β3 and/or β1 are knocked out, CHO cells that express recombinant integrins, and antibodies that recognize human integrins (e.g., β3, β1).

### Vectors

1. CRISPR pSpCas9(BB)-2A-GFP vector was obtained through Addgene (Ran, F. A., Hsu, P. D., Wright, J., Agarwala, V., Scott, D. A., and Zhang, F. (2013) Genome engineering using the CRISPR-Cas9 system. Nat Protoc 8, 2281-2308).

2. CD40L cDNAs (WT and mutant) in pCDNA3 were obtained from ATCC.

### Cells

1. HEK293, and CHO-K1 cells were obtained from ATCC.

2. HEK 293 cells, in which integrins β3 and/or β1 were generated in our lab.

3. CHO cells that express recombinant integrins were previously generated in our lab. They have been extensively tested in many laboratories including ours and published.

We perform short tandem repeat profiling using vendors (e.g., ATCC and Genetica) to identify the cell lines and to avoid cross-contamination. We test mycoplasma once per year using commercial kits. We keep cells in culture at most 1 month, discard them, and wake up new batch from frozen vials to avoid genetic drift.

We test mycoplasma infection of cells periodically using commercially available kits. If positive we treat them using antibiotics.

### Proteins

1. Proteins are prepared in our lab in E. coli and purified as soluble protein using affinity chromatography. They are >90% pure in SDS-gel electrophoresis. We check the size (by SDS-PAGE and staining) after synthesis. If protein size needs to be confirmed we re-sequence the expression plasmids. We validate the function of our WT GFs using commercial GFs.

2. Low endotoxin gelatin hydrogel is obtained from Nitta Gelatin (NC, USA).

### Antibodies

Antibodies are obtained commercially (e.g., Santa Cruz biotechnology).

### Mice

We study both males and females. Mice are obtained from commercial sources such as The Jackson Laboratory or Charles River Laboratories.

### Psoriasis-Like Model

Imiquimod (IMQ) is an agonist of toll like receptor (TLR) 7/8. Female and male mice (BALB/c or C57BL/6) at 8 to 10 wk of age are topically challenged with 62.5 mg of commercially available IMQ cream (5% Aldara; 3M Pharmaceuticals) or control vehicle cream (Vaseline) on the shaved back and the right ear for 7 consecutive days. Soluble CD40L is intraperitoneally administrated on the first day of IMQ challenge. The ear thickness is measured before the daily challenge. After a weeks' challenge, mice are euthanized and ears and back skin are collected for hematoxylin and eosin stain (H&E), and immunohistochemistry (IHC) and flow cytometry. The in vivo psoriasis models have been well characterized and are well suited for the proposed study relating to skin inflammation, which can only be performed in animals. Mice are used since in vivo inflammation models have been well established in mice.

### Narrative

We discovered that integrins bind to cytokines and form a triplex structure composed of integrin-cytokine-receptor (ternary complex model). Among the cytokines we studied is CD40L, involved in, e.g., inflammatory diseases. Our preliminary data demonstrated that we can use the ternary complex model to study CD40L/CD40 signaling, and to design new CD40L antagonists that are useful as novel drugs to treat inflammatory diseases (e.g., psoriasis) and other diseases.

### Example 2. Development of CD40L Mutant Polypeptides and Validation in an Atherosclerosis Model

### Abstract

CD40 binding to its cognate TNF superfamily ligands (i.e., CD40L/CD154) on activated T cells plays a key role in immune response and is a major target in inflammatory diseases. Anti-CD40L antibodies effectively suppress atherosclerosis in animal models; however, development of therapeutic anti-CD40L antibodies has been halted because they induced thromboembolisms in clinical trials. We recently discovered that several growth factors (e.g., FGF) directly bind to integrins and form a ternary complex (e.g., integrin-FGF-FGFR), indicating that integrins serve as a common co-receptor of growth factor signaling. Production of FGF mutants proved to be defective in integrin binding and subsequent outside-in signaling functioned as antagonists of cell activation. CD40L is known to bind to integrins such as α5β1 and can form a ternary complex with CD40. This leads to the scientific premise that signaling is mediated by ternary complex formation and that CD40L mutants defective in integrin binding act as antagonists in cell mediated signaling. Our preliminary data indicates that (a) CD40L binds to integrin Δvβ3 (a newly identified CD40L receptor), (b) CD40L mutants defective in binding to Δvβ3 and α5β1 act as antagonists to CD40L (CD40L decoy), and (c) the Δvβ3 and α5β1-binding site is located in the CD40L-CD40L trimeric interface, suggesting that this domain is cryptic in trimeric CD40L. We recently reported that several integrin ligands allosterically activate integrins by binding to a second allosteric site on the integrin (site 2). This second integrin binding domain was revealed by amino acid residues of CD40L involved that overlap with those involved in Hyper IgM syndrome (HIGMS1) that is caused by genetic defects in CD40L involved in immunoglobulin class switching. These findings lead to our understanding that loss of integrin binding is related to HIGMS1 through a mechanism of CD40L allosteric activation of Δvβ3 through binding to site 2 that elicits CD40L signaling. The goal of the experiments described in this example is to further elucidate the role of integrins in CD40/CD40L signaling and develop therapeutics for inflammation. We will: 1). Study the role of integrins in CD40L signaling. A) Study how missense mutations in HIGMS1 and the loss of integrin binding are related by testing the ability of missense HIGMS1 mutations to affect integrin binding. This will identify a cause of HIGMS1. B) Study how HIGMS1 mutants are antagonistic. 2). Determine the role of integrin outside-in signaling in CD40L signaling. A) Study how CD40L induces integrin-CD40L-CD40 ternary complex formation. B) Study the role of the integrin outside-in signaling in CD40L signaling. C) Characterize CD40L-induced allosteric activation of integrins. 3). Develop antagonistic CD40L mutants as therapeutics. A) Stabilize CD40L decoy by Fc fusion. B) Study the effect of CD40L decoys in a mouse model of atherosclerosis.

### Specific Aims

CD40-ligand (CD40L/CD154) plays a key role in immune regulation through binding to its receptor CD40 and its levels markedly increase in certain pathologic conditions (e.g., SLE). CD40L is a major target in inflammatory diseases. We recently discovered that several growth factors (e.g., FGF, IGF) directly bind to integrins and form integrin-growth factor-growth factor receptor ternary complexes. Integrins are common co-receptors of growth factor signaling (ternary complex model). Growth factor mutants that are defective in integrin binding are defective in signaling functions and act as antagonists. It has been reported that CD40L binds to integrins (e.g., α5β1) and that CD40 and α5β1 can simultaneously bind to CD40L. Thusly, CD40L/CD40 signaling fits well with the ternary complex model. We understand that CD40L signaling is mediated by ternary complex with integrin α5β1 and CD40, and that CD40L mutants defective in integrin binding act as antagonists during inflammation. Our preliminary studies found that (a) integrin Δvβ3 is a newly identified co-receptor for CD40/CD40L signaling, (b) the Δvβ3 and α5β1-binding site is located in the CD40L-CD40L trimeric interface, suggesting that integrin-binding site is not exposed in trimeric CD40L, and (c) the amino acid residue positions of CD40L involved in integrin binding overlap with those involved in the Hyper IgM syndrome type 1 (HIGMS1), which is caused by a genetic defect in CD40L that induces the defect associated with immunoglobulin class switching. This is consistent with the loss of integrin binding being related to HIGMS1. We recently reported that several integrin ligands allosterically activate integrins by binding to the allosteric site of integrin (site 2), which we recently discovered. In our preliminary studies, CD40L allosterically activated Δvβ3 by binding to site 2. Since mutations in several amino acid residues of CD40L involved in site 2 binding cause HIGMS1, it is understood that CD40L binding to site 2 and resulting activation of integrins are involved in CD40L signaling. CD40L mutants defective in binding to Δvβ3 and α5β1 are act as antagonists to CD40L. The goal of the experiments described in this example is to identify the role of integrins in CD40/CD40L signaling and establish a new model of CD40L/C40 signaling through pursuit of the following aims:

### 1. Determine the Role of Integrins in CD40L Signaling.

A) Study how HIGMS1 and the loss of integrin binding are related by testing the ability of HIGMS1 mutations to affect integrin binding. Several missense HIGMS1 mutations affect integrin binding, indicating that the ability to bind to integrins is required for CD40L signaling.

B) Study how HIGMS1 mutants are antagonistic in cell signaling of inflammatory processes.

2. Establish the Role of Integrin Outside-In Signaling Via a Ternary Complex with CD40L.

A) Study how CD40L induces integrin-CD40L-CD40 ternary complex formation. We use co-immunoprecipitation or pull-down assays to establish the ternary complex formation. Integrin Δvβ3 (and other integrins) form ternary complexes with CD40 and CD40L.

B) Study the role of the integrin outside-in signaling in CD40L signaling. We use integrin β1 or β3 cytoplasmic mutations that block outside-in signaling from the extracellular matrix. This integrin signaling pathway plays a key role in CD40L signaling as well.

C) Characterize CD40L-induced allosteric activation of integrins. Since amino acid residues of CD40L for site 2 binding are also involved in HIGMS1, we examine how the site 2 binding is also involved in CD40L signaling.

### 3. Develop Antagonistic CD40L Mutants as Therapeutic for Atherosclerosis

A) Stabilize CD40L decoys by Fc fusion.

B) Study the effect of CD40L mutants in a mouse model of atherosclerosis.

The work described in this example enhances our understanding of the mechanisms of CD40L/CD40 signaling and furthers the development of CD40L decoys as antagonists to CD40L signaling.

### Significance

Direct integrin binding and ternary complex formation is required for several growth factors. Integrin antagonists inhibit insulin-like growth factor (IGF1) and fibroblast growth factor-2 (basic FGF, FGF2) signaling (1,2). This indicates that integrins are involved in growth factor signaling through crosstalk. However, current models of integrin-growth factor crosstalk propose that integrins contribute to growth factor signaling almost exclusively through integrin binding to the extracellular matrix (ECM), and that growth factors only bind to their cognate receptors and two independent signals merge inside the cells (3-5). We have reported that several cytokines (FGF1, IGF1, neuregulin-1, and fractalkine) directly bind to integrins and this interaction leads to ternary complex formation (integrin-growth factor-receptor tyrosine kinase), which is required for their signaling functions (6-12). Notably, ternary complex formation is seen in several different growth factors. Recently, we found that direct binding of integrins to IL-1β plays a critical role in IL-1β signaling (13). This indicates that crosstalk between integrins and growth factor receptors through direct integrin binding is a common mechanism in growth factor signaling. Mutants defective in integrin binding to these growth factors are functionally defective and suppress signaling induced by WT growth factor (dominant-negative antagonists) (9-11).

CD40L is a key immunomodulatory factor and a major therapeutic target. CD40 is a cell surface receptor that belongs to the tumor necrosis factor-R (TNF-R) family that was first identified and functionally characterized on B lymphocytes (14). Its critical role in T cell-dependent humoral immune responses was demonstrated by patients with the hyper-IgM syndrome type 1 (HIGMS1), as well as by gene targeting in mice. However, in recent years it has become clear that CD40 is expressed much more broadly, including expression on monocytes, dendritic cells, endothelial cells, and epithelial cells. In addition, CD40L is also expressed more widely than on activated CD4+ T cells alone (15). Therefore, it is now understood that CD40-CD40L interactions play a more general role in immune regulation.

CD40L is a type II protein ligand member of the tumor necrosis factor (TNF) superfamily that, via interaction with CD40, is a key immunomodulatory factor responsible for modulating nearly all aspects of the adaptive immune response. CD40L is expressed as a transmembrane form and released as a soluble form (sCD40L) by proteolytic cleavage. CD40L/CD40 interaction is required for enhancing antigen presenting functions of dendritic cells, macrophages, and B cells; maturation of humoral responses; and enhancement of effector T cell responses (16). Additional functions of CD40L include the initiation of inflammatory and procoagulatory responses in vascular endothelial cells (17-19). CD40L is a key player in chronic autoimmune inflammatory diseases, including systemic lupus erythematosus (SLE), diabetes, chronic kidney disease (20, 21), and in development and progression of atherosclerosis (22, 23). Based on striking efficacy in preclinical models, clinical trials using humanized or chimeric anti-CD40L monoclonal antibodies blocking CD40/CD40L interactions were undertaken in early 2000s. However, progress was halted due to the incidence of thromboembolic events in clinical trials.

It is believed that trimeric CD40L is biologically active, but monomeric CD40L is not. CD40L levels markedly increase in certain pathologic conditions (SLE and RA), but CD40L exists mainly in a monomeric form (24).

CD40L simultaneously binds to integrin α5β1 and CD40. The contribution of integrins to CD40L/CD40 signaling has been largely ignored (25, 26). It has been reported that CD40L stabilizes arterial thrombi through binding to integrin αIIbβ3 (27). αIIbβ3 recognizes the KGD motif at the N-terminus of CD40L (residues 115-117 of CD40L). It has also been reported that CD40L binds to integrin α5β1 and transduces signals through this integrin in a CD40- and αIIbβ3-independent manner. It has been shown that CD40 and integrin α5β1 can bind to CD40L simultaneously (28). These integrins recognize CD40L differently. α5β1 does not require the KGD motif, and its binding site in CD40L is unclear. Mutations of the CD40-binding site (Y145A, R203A, or Y145A/R203A double mutant) did not affect α5β1-CD40L interaction (28), suggesting that α5β1 and CD40 can co-exist on CD40L. However, the specifics of integrin-CD40L interaction are unclear.

Integrin Δvβ3 is a novel CD40L receptor, and the integrin-binding site in CD40L is located at the trimeric interphase (our preliminary studies). In our preliminary studies, we identified integrin Δvβ3 as a novel receptor for CD40L and found that Δvβ3 does not recognize the N-terminal KGD motif of CD40L. In a previous study, it has been reported that Δvβ3 does not bind to CD40L, which could be accounted for by Δvβ3 remaining unactivated in that study (28). We predicted the putative integrin-binding site in CD40L using docking simulations and by introducing mutations in the predicted integrin-binding site in CD40L. Notably, we found that the Δvβ3-binding site is located in the trimerization interface of CD40L, indicating that the Δvβ3-binding site is not exposed in the CD40L trimer, but exposed in the monomer. This indicates that CD40L monomer plays a potential role in CD40L signaling. We address this hypothesis in subaim #1.

CD40L allosterically activates integrins by binding to allosteric ligand binding site of integrins (site 2) that we recently identified (our preliminary studies). Integrins are generally believed to be activated by inside-out signaling. We recently, however, discovered that integrins can be activated in an allosteric mechanism. We recently identified two integrin ligands, fractalkine/CX3CL1 and secreted phospholipase A2 type IIA (sPLA2-IIA) that bind to the classical RGD-binding site (site 1) of integrin Δvβ3 (12, 29). Notably, fractalkine and sPLA2-IIAs activated integrins Δvβ3, α4β1, and α5β1 by binding to an additional ligand-binding site (site 2) in an allosteric manner in the absence of their receptors (e.g., CX3CR1 for fractalkine) (30, 31). The peptide from site 2 of β3 directly bound to fractalkine and sPLA2-IIA, and suppressed integrin activation by fractalkine and sPLA2-IIA, indicating that they directly bind to site 2 and mediate integrin activation (30, 31). We recently found that stromal cell derived factor-1 (SDF1) allosterically activates these integrins by binding to site 2 (32). These findings indicated that allosteric activation of integrins by binding to site 2 is also be a common mechanism of integrin activation.

Our preliminary studies found that CD40L allosterically activated integrin Δvβ3 by binding to the allosteric binding site (site 2). This will further the development of a new signaling mechanism of CD40L/CD40/integrin signaling. Notably, CD40L binds to site 1 (the classical RGD-binding site) and site 2 (allosteric site) in a different manner. Thus, CD40L mutants that specifically block site 1 binding (e.g., Y170E) and site 2 binding (e.g., K143E) are excellent tools for studying the properties of site 2 and CD40L signaling. This is distinct from our previous studies, which found that fractalkine and sPLA2-IIA bind to site 1 and site 2 in a similar manner. For example, the same mutations of fractalkine suppress fractalkine binding to both site 1 and site 2.

Integrin binding to CD40L and genetic diseases HIGMS1 (our preliminary studies). In our preliminary studies, we located integrin-binding sites of CD40L using docking simulations and mutagenesis. Amino acid residues of CD40L that are involved in integrin binding overlap with those that have been reported to be mutated in HIGMS1 patients, indicating that the loss of integrin binding is related to HIGMS1. Consistently, mutating the amino acid residues at the integrin-binding interface of CD40L suppressed integrin binding and signaling functions of CD40L. Interestingly, the amino acid residues involved in site 2 binding are distinct from those for site 1 binding and reported to be mutated in HIGMS1 patients. This is consistent with the model that HIGMS1 (defective CD40L signaling) is induced by the loss of integrin binding to CD40L (in addition to the effect of mutations on the trimerization and protein structure).

Antagonistic CD40L mutants as potential therapeutics (our preliminary studies). The observation that α5β1 and CD40 can simultaneously bind to CD40L (28) is consistent with the model that they form a ternary complex. Based on our previous studies, we understand that ternary complex (integrin-CD40L-CD40) formation on the cell surface and the integrin binding to CD40L is critical for CD40/CD40L signaling, and that the CD40L mutants defective in integrin binding are defective in signaling and act as antagonists. In our preliminary studies, we discovered that the CD40L (monomeric) mutants that are defective in integrin binding act as antagonists of CD40L signaling. Thus, the CD40L mutants are useful as therapeutics for chronic inflammation. The studies described in this example enhance our understanding of the role of integrins in CD40L/CD40 signaling and identify novel therapeutic targets. We stabilize CD40L decoy and study its efficacy in an animal model of atherosclerosis (subaim #3). We also study how integrins contribute to CD40L signaling (subaim #2).

A wealth of published data supports a central role for CD40-CD40L interactions in the pathogenesis of atherosclerosis. CD40L deficiency or pharmacological inhibition of CD40L in ApoE^{-/-} mice results in the development of stable atherosclerotic plaque. CD40 and CD40L protein are present in a variety of cell types in human atherosclerotic lesions and also in mouse models of atherosclerosis. We understand that signaling via CD40-CD40L in endothelial cells and monocytes promotes early plaque formation. Further, the role of CD40-CD40L is more predominant in advanced, rupture-prone, and ruptured plaques. Since acute complications of atherosclerosis are the result of plaque rupture, CD40L inhibition provides an effective novel therapeutic approach to prevent atherosclerotic plaque progression and plaque rupture.

### Preliminary Studies

The purpose of these preliminary studies was to determine that CD40L/CD40 signaling involves ternary complex formation (see significance), as we observed for several other growth factors, and that mutants defective in integrin binding are antagonistic. Unexpectedly, we found that the loss of integrin binding to CD40L is related to HIGMS1.

Integrin αvβ3 binds to CD40L in a KGD-independent manner. It has been reported that integrin αIIbβ3 recognizes the N-terminal KGD motif of CD40L (residues 115-117) (33), and that the N151A/Q166A mutations on the surface of trimeric CD40L are involved in α5β1 binding (34). Also, it has been reported that CD40L binds to integrin α5β1 but not to integrin Δvβ3 (35). Our preliminary docking simulation studies predicted that CD40L monomer binds to integrin Δvβ3 well (see below), indicating that Δvβ3 binds to the trimeric interface of CD40L, not to trimer surface. We studied if CD40L binds to integrin Δvβ3 using sCD40L (residues 118-262) that has no KGD motif at the N-terminus. We immobilized WT CD40L (with no KGD, Δ115-117) to wells of a 96-well microtiter plate and incubated with soluble Δvβ3 in Tyrode/Hepes buffer (+1 mM MnCl₂ to fully activate Δvβ3) for 1 h, and bound Δvβ3 was quantified using anti-β3 antibody (AV10). We found that soluble Δvβ3 bound to immobilized sCD40L in ELISA-type binding assays in a dose-dependent manner (FIG. 1A). This indicates that integrin Δvβ3 is a new CD40L receptor that does not require the KGD motif for binding to sCD40L.

The integrin-binding site is located in the trimerization interface. We studied how CD40L binds to integrin Δvβ3 using docking simulations between monomeric CD40L that lacks the KGD motif (PDB code 1ALY) and the headpiece of Δvβ3 (PDB code 1L5G), which has an open headpiece conformation. Twenty out of 100 dockings grouped in the first cluster (docking energy −24.5 kcal/mol), (FIG. 1B). The simulation predicted that monomeric CD40L binds to the RGD-binding site of Δvβ3 (designated site 1). The simulation predicted that the integrin-binding site in CD40L is located in the trimerization interface (FIG. 1C). It has recently been reported that α5β1 does not require the KGD motif and that the combined N151A/Q166A in CD40L reduces α5β1 binding (34). N151 and Q166 are not located in the predicted integrin-binding site in our preliminary studies. We found that α5β1 and Δvβ3 bind to CD40L are both located in the trimeric interface of CD40L, not on the trimer surface exposed to solvent (see below).

Based on the prediction, we selected several amino acid residues within the predicted integrin-binding site for mutagenesis (Tyr170, His224/Gly226, and Gly252). Notably, Tyr170 and His224, Gly226 have been reported to be mutated in HIGM1, a family of genetic disorders in which the level of IgM is relatively high as a result of a defect in CD40L signaling.

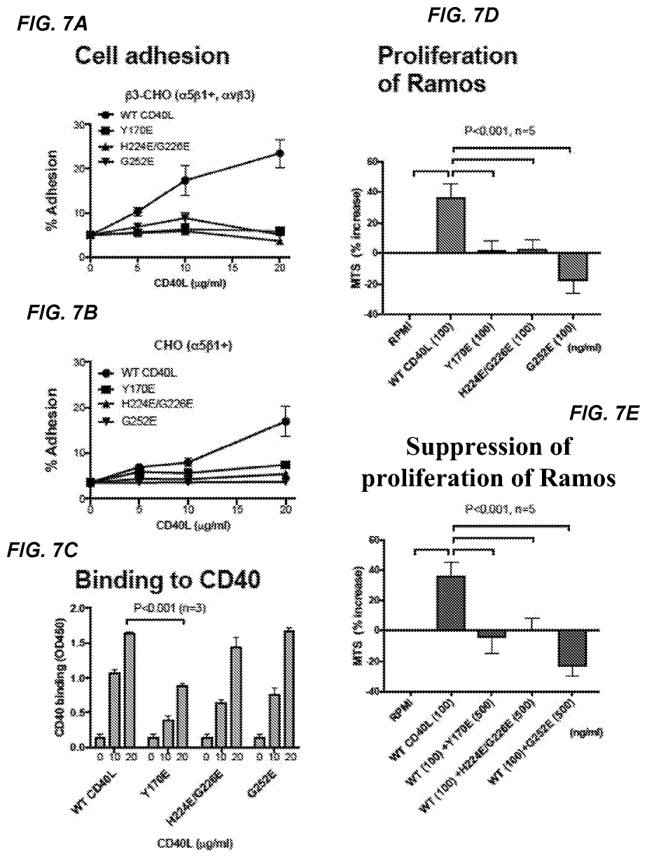

We studied the ability of the Y170 to E (Y170E), H224E/G226E, and G252E mutants to bind to Δvβ3 using CHO cells (CD40-negative) that express recombinant Δvβ3 (β3-CHO cells). Wells of a 96-well microtiter-plate were coated with WT and mutant CD40L and remaining protein binding sites were blocked with BSA. CHO cells and β3-CHO cells were added to the wells in DMEM and incubated for 1 h at 37° C. Bound cells were quantified using endogenous phosphatase activity (FIGS. 7A and 7B). We found that the Y170E, H224E/G226E, and G252E mutations significantly reduced binding to both Δvβ3 and α5β1 in adhesion assays. These results are consistent with the docking model. These findings indicate that the binding site for Δvβ3 and α5β1 is cryptic in CD40L trimer and exposed in the monomeric form. Our results also indicate that integrin binding of CD40L is defective in patients in which these amino acid residues in the trimeric interface are mutated.

Also, CD40L supports adhesion of CHO cells that express recombinant Δvβ3 (β3-CHO cells, CD40−, α5β1-negative, Δvβ3-positive) better than CHO cells (α5β1-positive, Δvβ3-negative). These findings led to our conclusion that αvβ3 is a new CD40L receptor. Our results thus demonstrated that monomeric CD40L, not trimeric CD40L, binds to integrins Δvβ3 and α5β1. Our docking model predicted that the one or two CD40 molecules bind to the monomeric CD40L/integrin complex, resulting in the CD40/CD40L monomer/integrin ternary complex. This is consistent with the previous report that α5β1, and CD40 can simultaneously interact with CD40L (28). It has been reported that CD40L levels markedly increase in certain pathologic conditions (e.g., SLE), and that CD40L exists mainly in a monomeric form (24). It has been believed that trimeric CD40L is biologically active, but monomeric CD40L is not (36). Therefore, the role of monomeric CD40L in the pathogenesis of inflammatory diseases has been ignored.

We understand that CD40L induces ternary complex formation (integrin-CD40L-CD40) on the cell surface, and that integrins and CD40 work together in CD40L, as in several other growth factors (see Significance). If ternary complex formation is critical for CD40L signaling, CD40L mutants defective in integrin binding will be defective in signaling and have inhibitory function. We studied how WT and mutant sCD40L affect proliferation of human B-cells or B-cell leukemia (FIG. 7C). Ramos cells were incubated with WT CD40L (100 ng/ml) or mutant CD40L for 48 h in RPMI (serum-free) and cell viability was determined by MTT assays. In competition assays WT CD40L (100 ng/ml) and mutant CD40L (500 ng/ml) were added (FIG. 7D). We found that WT sCD40L enhanced proliferation, but mutant CD40L did not. Furthermore, mutant sCD40L reduced proliferation enhanced by WT sCD40L (FIG. 7E), demonstrating that they are antagonistic.

### Innovation

1. Identified integrin Δvβ3 as a new CD40L receptor.

2. Discovered that the binding site of CD40L for αvβ3 and α5β1 is located in the trimeric interface (not exposed to the surface in trimeric CD40L). We understand that monomeric CD40L binds to αvβ3 and α5β1, which are thereby biologically active. We also understand that monomeric CD40L, which is elevated in serum of SLE patients, is related to inflammation associated with onset of atherosclerosis.

3. Discovered that the CD40L mutants that are defective in binding to αvβ3 and α5β1 are defective in signaling and are antagonistic.

4. Discovered that CD40L allosterically activates soluble integrin Δvβ3 in solution by binding to site 2 (allosteric binding site).

5. Amino acid residues of CD40L involved in integrin binding are mutated in HIGMS1. We understand that the loss of integrin binding is related to the defect in CD40L in HIGMS1.

### Approach

### 1. Determine the Role of Integrins in CD40L Signaling.

### Preliminary Studies

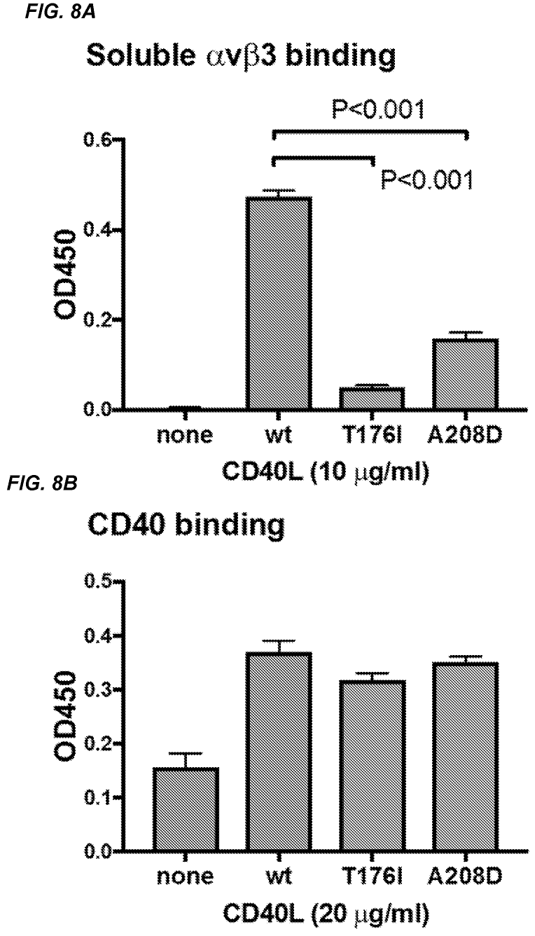

We introduced two HIGMS1 mutations in the trimeric interface into CD40L and expressed proteins using PET28a in E. coli as described above. We found that the CD40L mutants were defective in integrin binding as expected, while they bound to CD40 well (FIG. 8). These findings are consistent with the idea that HIGMS1 mutations is defective in signaling at least in part because they are defective in integrin binding.

A) Establish how HIGMS1 and the loss of integrin binding are related. The goal of this subaim is to establish how the loss of integrin binding to CD40L is related to HIGMS1 (due to loss of CD40L signaling). Many missense CD40L mutations in HIGMS are located in the trimerization interface. We thus understand that the loss of integrin binding to CD40L is related to HIGMS1. To address this question, we test the ability of all known HIGMS1 mutants in the integrin-binding interface to bind to integrins. We study if several other HIGMS1 mutants (Y170C (37), Q174R (38), T1761, A208D, H224Y, G226A, G227V (39), and L258S (37), see, FIG. 5) are defective in integrin binding and antagonistic (these CD40L mutants and other references are listed at www.uniprot.org/uniprot/P29965). This enhances understanding of the mechanism of the genetic defects. We introduce individual HIGMS1 mutations in the trimeric interface to CD40L, synthesize mutant proteins, and determine the ability of the mutants to bind to integrins in adhesion assays and binding ELISA-type assays as in our preliminary studies. We determine the signaling functions of the mutants using NF-kB reporter assays and anti-apoptotic assays. We confirm that the mutants still bind to CD40.

CD40/NF-kB reporter assays: We stably express CD40 and NF-kB reporter (luciferase) in HEK293 cells and measure luciferase activity after CD40L stimulation. We include an IL-1R antagonist and anti-TNF-α to block NF-kB activation by IL-1β or TNF-α. This HEK293 reporter system is also commercially available. To avoid NF-kB activation by IL-1β or TNF-α we include an IL-1 receptor antagonist (ILRN) and antibody to TNF-α in cell culture. We have effectively used NF-kB activation by IL-1β in recent studies (13).

Caspase-8 activation: Jurkat T cells are pre-treated or not with sCD154 for 6 hrs at 37° C. and stimulated with the anti-Fas (CH-11) antibody or isotype-matched control (IgM) for 3 hrs at 37° C. Cells are then boiled for 7 min and caspase-8 cleavage is assessed by immunoblot using specific antibodies to caspase-8. Full-length caspase-8 (57 kd) is cleaved into p44/p43 and P18 fragments upon activation. These assays evaluate the ability of the sCD40L mutants to Fas-induced increase cell death or compete with native CD40L and suppress the levels of cell death protection and caspase-8 activation (40).

CD40 binding assays: We coat wells of 96-well microtiter plates with CD40L (WT and mutants) and incubate with soluble CD40 fused to Fc (available in our lab, also commercially available). Bound CD40 is measured using anti-Fc antibodies.

Proliferation and cytokine production in B-lymphocytes from mouse spleen: B-lymphocytes are prepared from splenocytes from WT mice by magnetic separation. Isolated B-lymphocytes are stimulated with sCD40L (WT CD40L or HIGMS1 mutants) (up to 100 ng/ml) and cell proliferation by MTS is assayed or by BrdU incorporation and measure cytokine production (TNFα and MIP-1α) by ELISA. These studies confirm that WT sCD40L induces B cell proliferation (up to 100 ng/ml), but HIGMS1 mutants do not.

B) Study antagonistic properties of HIGMS1 mutants. Our preliminary studies showed that CD40L mutants defective in integrin binding are antagonistic (CD40L decoy). At least two HIGMS1 mutants are defective in integrin binding (our preliminary studies, FIG. 8). Here, we assess the antagonistic properties of HIGMS1 mutants, and the extent to which HIGMS1 and the loss of integrin binding and subsequent defective class switching from IgM to IgG are related. We study how HIGMS1 mutants suppress cell proliferation or cytokine production induced by WT CD40L in NF-kB activation or B-cell proliferation induced by WT CD40L (as described above).

If needed, we introduce HIGMS1 mutations into full-length CD40L and stably express in CHO cells and study how the mutant CD40L is defective in signaling functions. We co-culture mouse splenocyte B cells with fixed CHO cells that express CD40L as described (41).

The experiments described here determine how HIGMS1 mutations in the trimeric interface are defective in integrin binding and thereby lead to defects in signaling functions. This follows from our preliminary studies, in which two HIGMS1 mutants were defective in integrin binding but intact in CD40 binding (FIG. 4). Consistent with the model, these HIGMS1 mutants are antagonistic. These studies also further enhance the understanding of HIGMS1 pathogenesis. While the HIGMS1 mutations are understood to dissociate active CD40L trimer to non-functional monomer, monomeric CD40L may also be functional. If this is the case, the effect of HIGMS1 mutations is primarily on integrin binding.

### Summary

We determine how the loss of integrin binding is related to HIGMS1. Additional studies further explore how integrin crosstalk influences CD40L regulation of immunoglobulin class switching in B cells.

### 2. Establish the Role of Integrin Outside-In Signaling in CD40L Signaling.

A) Further understand the process by which CD40L induces integrin-CD40L-CD40 ternary complex formation. Integrin α5β1 and CD40 can simultaneously bind to CD40L in competition assays (28), but it has not been biochemically tested how CD40L induces integrin-CD40L-CD40 ternary complex. We study how CD40L induces co-precipitation of integrins (α5β1 or Δvβ3) with CD40.

Biochemical detection of the ternary complex: We incubate B cell lymphoma cell (e.g., Ramos cells) with WT CD40L (His-tagged) and purify integrins or CD40 from cell lysates by immune precipitation or pulled down using Ni-NTA Sepharose. Purified materials are analyzed by Western blotting using specific antibodies to CD40 or integrins to determine the extent to which CD40 and integrins are co-purified. We then use CD40L mutants defective in integrin binding (see our preliminary studies) to verify that the CD40L mutants do not associate with integrins. Alternatively, we use purified soluble integrin (e.g., Δvβ3 or α5β1) and soluble CD40 (available in our lab). These proteins are mixed and incubated with WT or mutant CD40L and subjected to immunoprecipitation using anti-integrin or anti-CD40 antibodies or to pull-down using Ni-NTA Sepharose. The integrin-CD40L-CD40 ternary complex will include signaling components downstream of the complex that are involved in CD40L/CD40 signaling.

B) Study the Role of the Integrin Outside-In Signaling in CD40L Signaling.

### Research Design

The role of integrin cytoplasmic tail: Here we further enhance our understanding of how CD40L binding to integrins Δvβ3 and α5β1 induces signals that are common to other known integrin ligands (e.g., fibrinogen and fibronectin). Previous studies showed that IL2R (the extracellular and transmembrane domains that are fused with the cytoplasmic domains of integrin β1 subunit (IL2R-β1 tail) block the integrin-mediated signaling from ECM when expressed on the cell surface (42). We first construct IL2R-β1 tail in a pCDNA3.1 expression vector, and study how the IL2R-β1 tail disturbs CD40L signaling in Jurkat T cells. We measure the CD40L signaling as described above. Following confirmation that the integrin β1 tail disrupts the CD40L/CD40 signaling, we generate several truncation mutants of β1 with different lengths of the cytoplasmic tail, and test how truncated IL2R-β1 affects CD40L signaling in Jurkat cells. This allows determination of the position of the β1 tail that is involved in CD40L signaling. A number of integrin-binding proteins can be involved in CD40L signaling, since at least 42 “adaptor proteins” have been reported to bind to the β1 cytoplasmic tail (43). The first “hot spot” is a membrane proximal HDRK/HDRR motif that binds to Src-family kinase Fyn, FAK (focal adhesion kinase), paxillin, and skelmin. The second and third hot spots are a membrane-proximal NPxY motif and a membrane-distal NxxY motif. Both motifs are recognition sites for phosphotyrosine-binding (PTB) domains and almost all the adaptors that bind to these motifs do so via PTB domains. Therefore, mutation of the β1 cytoplasmic tail of these hot spots allows identification of the integrin-binding proteins that are involved in CD40L signaling. We perform similar studies using the IL2R-β3 tail in Jurkat cells to confirm that the IL2R-β3 tail suppresses CD40L signaling because integrin-binding proteins common to β1 and β3 are involved in CD40L signaling.

Role of GNA13 in outside-in signaling: Integrin outside-in signaling from ECM ligands is understood to be involved. Previous studies showed that G protein α13 (GNA13) directly binds to the integrin β1 cytoplasmic domain upon ligand binding, and mediates “outside-in signaling” from extracellular matrix (ECM) ligand fibrinogen in integrin aIIbβ3 (44). Interestingly, amino acid residues 731-733 (EAE) of the β1 cytoplasmic domain are conserved in several integrin β subunits and are critically involved in GNA13 binding to β1. Mutating 731-733 EAE to AAA (the 731-733AAA mutant) suppresses GNA13 binding (45). We first study how the β1 AAA mutation in the IL2R-β1 tail affects CD40L signaling in Jurkat cells. We also study how CD40L activates NF-kB in Jurkat cells in response to CD40L as described (46). We measure cell proliferation (e.g., MTS assays and BrdU incorporation) as described (6). These experiments establish the role of the EEE motif (and therefore GNA13 binding) in CD40L signaling. Finding that the β3 the 731-733AAA or corresponding β1 mutant is defective in mediating CD40L signaling would indicate that the outside-in signaling pathway from integrin ligands, including GNA13-binding to the β cytoplasmic domain, plays a role in CD40L signaling as well.

Following confirmation that GNA13 is involved downstream of integrins in CD40L signaling, we next study what signaling molecules are located downstream of GNA13.

If necessary, we study whether any other cytoplasmic proteins bind to the β3 cytoplasmic tail (see below).

Regions of β3 cytoplasmic domain other than the EAE motif (see above) may be involved in CD40L signaling. We identify such regions using truncation mutants of the β3 tail, and subsequently identify adaptor proteins involved in CD40L signaling. In the current model, integrin-ligand binding induces conformational changes in integrins and α and β cytoplasmic tails are separated as a result (outside-in signaling). Mapping regions in the β3 cytoplasmic tail that are involved in CD40L signaling provides useful information on the proteins involved in CD40L signaling. We have generated truncation mutants of the β3 cytoplasmic domain by introducing stop codons at positions 718, 725, 729, 737, 760, and 761 to remove hot spots. β3 mutants are transiently expressed in β3-knockout HEK293 cells in a mammalian expression vector. β3 mutants are in the form Δvβ3 (hamster αv/human β3 hybrid, this integrin is functional) together with human CD40. The ability of β3 mutants to mediate CD40L signaling is determined as described above. We determine the levels of β3 expression (median fluorescent intensity or positive cells) in flow cytometry, and CD40L signaling using an NF-kB reporter. Once we identify the region of β3 tail that is involved in CD40L signaling, we mutate individual amino acids in the region of the human β3 cytoplasmic tail (hot spots) that are critical for CD40L signaling (identified by experiments using truncation mutants). These mutations include Tyr or Thr/Ser phosphorylation sites. We measure the effect of individual mutations as described above. We use CD40L decoy and antagonists to Δvβ3 as negative controls to ensure that Δvβ3-CD40L interaction is involved.

C) Characterize CD40L-Induced Allosteric Activation of Integrins.

### Preliminary Studies

sCD40L allosterically activates integrins by binding to site 2. It is generally believed that integrins are solely activated by inside-out signaling. However, we recently discovered that integrins can be activated by their own ligands in an allosteric mechanism. We recently identified fractalkine and sPLA2-IIAs bind to the classical RGD-binding site (site 1) (12, 29). Notably, fractalkine and sPLA2-IIAs activated integrins by binding to an additional ligand-binding site (site 2) in an allosteric manner (30, 31). We studied how sCD40L enhances the binding of soluble Δvβ3 to its specific ligand γC399tr (binds site 1) that was immobilized in cell-free conditions (FIG. 3A). Wells of a 96-well microtiter plate were coated with γC399tr (50 μg/ml in PBS) for 2 hrs at room temperature and incubated with soluble Δvβ3 (5 μg/ml) and sCD40L for 1 hour in Tyrode/HEPES with 1 mM Ca²⁺ (which keeps Δvβ3 in an inactive form). Bound Δvβ3 was quantified using anti-β3 antibody (AV10). This demonstrated that sCD40L activates Δvβ3 in an allosteric manner. CD40L is a transmembrane protein, and soluble CD40L binds to cell surface proteoglycans. Our preliminary studies showed that we need high concentrations of soluble CD40L to activate soluble integrin Δvβ3, but we need much less CD40L to activate integrins on the cell surface since CD40L is highly concentrated on the cell surface. We previously reported that the peptide from site 2 of β3 (QPNDGQSHVGSDNHYSASTTM, residues 267-287 of β3, Cys-273 is changed to S, fused to GST) directly bound to fractalkine and sPLA2-IIA and suppressed integrin activation by fractalkine and sPLA2-IIA, indicating that they directly bind to site 2 and mediate integrin activation (30, 31). We studied if CD40L binds to site 2 peptides in ELISA-type binding assays. Wells of a 96-well microtiter-plate were coated with CD40L and incubated with site 2 peptide fused to GST in PBS/0.05% Tween20 for 1 hr. Bound GST was measured using anti-GST (FIG. 3B). We found that site 2 peptide bound to sCD40L, indicating that sCD40L binds to site 2. We found that Y170E, H224E/G226E, and G252E CD40L mutants, which do not bind to site 1 well (FIG. 7), activated Δvβ3 (FIG. 3C), demonstrating that CD40L binds to site 1 and site 2 in a different manner. To predict how CD40L binds to site 2, we performed docking simulations of the interaction between CD40L (1ALY.pdb) and the Δvβ3 headpiece (1JV2.pdb, closed-headpiece), (FIG. 3D). The simulation predicted that monomeric CD40L binds to site 2 well (docking energy −20.5 kcal/mol). The site 2-binding interface was predicted to be distinct from that of site 1. The predicted site 2-binding interface of CD40L included Glu129, Lys143, Glyl44, and Leu155, which have been reported to be mutated in HIGMS1. Mutating these amino acid residues will suppress binding of CD40L to site 2, and as a result suppress allosteric activation of integrins. Site 2 is located on the opposite side of site 1 in the headpiece of integrins (FIG. 4). Allosteric activation is a newly discovered mechanism of integrin activation. It has previously been understood that integrins are activated primarily by inside-out signaling. Although we have used relatively high concentrations of sCD40L to activate soluble integrins, since CD40L is expressed as a transmembrane protein on the cell surface, biological concentrations of transmembrane form CD40L will be able to activate integrins. Since CD40L binds to site 1 and site 2 differently, we employ CD40L mutants that bind only to site 2 (e.g., Y170E) and those that probably bind only to site 1 (e.g., K143T). These tools are quite useful to analyze the binding kinetics and biological role of site 2. It is also understood that mutations in the site 2 binding interface of CD40L induce biological CD40L functional defects, since some mutations of several amino acid residues in the site 2 binding interface (e.g., Glu129, Lys143, Glyl44, Leu155) are involved in HIGMS1.

### Rationale

Previous studies showed that integrins are allosterically activated by the binding of ligands to another ligand binding site (site 2). This concept has been validated in several different ligands and integrins. Fractalkine and sPLA2-IIAs not only bound to the classical RGD-binding site (site 1) of integrin Δvβ3 (12, 29), but also to activated integrins Δvβ3, α4β1, and α5β1 by binding to site 2 in an allosteric manner (30, 31). The peptides from the amino acid sequence of site 2 of β3 directly bound to these ligands and suppressed integrin activation by them, demonstrating that they directly bind to site 2 and mediate integrin activation (30, 31). Also, stromal cell derived factor-1 (SDF1) allosterically activates these integrins by binding to site 2 (32). These findings indicate that allosteric activation of integrins by binding to site 2 is a common mechanism of integrin activation. Our preliminary studies found that sCD40L enhanced the binding of Δvβ3 to its specific ligand γC399tr (a fragment of fibrinogen) in cell-free conditions in a dose-dependent manner. Also, monomeric CD40L was predicted to strongly bind to site 2 in docking simulations (docking energy −20.5 kcal/mol). This indicates that sCD40L activates Δvβ3 in an allosteric manner. Interestingly, our preliminary studies demonstrated that CD40L binds to site 1 and site 2 differently (FIG. 8). The simulation predicted that Glu129, Lys143, Glyl44, and Leu155 are involved in site 2 binding. These amino acid residues are mutated in HIGMS1 (CD40L signaling is defective). Therefore, we understand that the binding of CD40L to site 2 is also involved in CD40L signaling functions.

### Research Design

Here, we study how CD40L mutations in the predicted site-2 binding site (e.g., Glu129, Lys143, Glyl44, Leu155) affect CD40L signaling and/or integrin activation using Ramos cell proliferation and NF-kB reporter assays. We understand that the CD40L binding to site 2 is also involved in CD40L signaling. Also, CD40L mutants are useful tools to study the role of site 2 in detail, since CD40L mutants distinguish two integrin binding sites (site 1 vs site 2). We measure binding kinetics of CD40L to site 1 or site 2 using mutants that bind to site 1 or site 2 specifically.

The experiments described here establish that CD40L can allosterically activate integrins. The observation that the site 2 binding site of CD40L contains several HIGMS1 mutations is consistent with the model that the defect in this function leads to HIGMS1.

### Summary

Here, we establish that CD40L induces integrin-CD40L-CD40 ternary complex formation. We study the role of the integrin outside-in signaling in CD40L signaling using integrin β1 or β3 cytoplasmic mutations that block outside-in signaling from the extracellular matrix. We also characterize CD40L-induced allosteric activation of integrins.

### 3. Development of Antagonistic CD40L Mutants as Therapeutics for Atherosclerosis.

In our ternary complex model of growth factor signaling (see Significance), growth factor mutants defective in integrin-binding (defective in signaling function) still bind to their cognate receptor, and thereby compete with WT growth factor for receptor binding (decoys). In our preliminary studies, several CD40L mutants defective in integrin binding (e.g., G252E) showed an antagonistic effect in cell proliferation assays (FIG. 7D). Here, we select one with the strongest antagonistic activities for further development using reporter assays and signaling assays (A). We stabilize CD40L decoys by Fc fusion (A). Alternatively, we use myristoylation. We study their anti-inflammatory activities in a mouse model of atherosclerosis (47, 48) (B). The experiments describe here establish that the CD40L decoys retain anti-inflammatory effects in vivo. Since integrins Δvβ3 and α5β1 and CD40 are widely expressed in vascular cells (see significance), CD40L decoys will suppress CD40L/CD40 signaling in many different cell types (including monocytes, dendritic cells, endothelial cells, and epithelial cells).

(A) Development of Fc Fusion Protein of CD40L Decoy.

Fc fusion is a well-established standard strategy for protein stabilization. Wild-type CD40L fused to the N-terminus of human or mouse Fc has been commercially available (laboratory use only) and shown to be functional in inducing proliferation of mouse splenocyte B-cells.

Here, we generate CD40L decoy as an Fc fusion protein. CD40L decoys (residues 118-262) are fused to the N-terminus of mouse Fc using a pFUSE-mFc vector (Invivogen) with a linker peptide [20 amino acids, (GGGGS)×4]. CD40L decoy-Fc is expressed in mammalian cells (CHO or HEK293 cells) and secreted into culture media. CD40L decoy-Fc is purified from media using protein A affinity chromatography. We generate wild-type CD40L (residues 118-262) as Fc fusion protein as a positive control to verify the function of Fc protein.

Proliferation of B cells: We study the ability of CD40L decoy-Fc to induce proliferation of mouse splenocyte B cells (as described above). Wild-type CD40L-Fc will induce cell proliferation in a dose-dependent manner, but CD40L decoy will not. Also, CD40L decoy-Fc will suppress cell proliferation induced by wild-type CD40L-Fc.

Half-life of CD40L decoy Fc: We determine the half-life of CD40L decoy Fc by labeling them (with I¹²⁵) and injecting it into mice. We take blood samples and measure the levels of radioactivity. CD40L-Fc will have much longer half-life than unmodified CD40L.

Mouse CD40L decoys. In the event that human CD40L decoy is immunogenic in mice, we design mouse CD40L decoy-mouse Fc to reduce immunogenicity by introducing human-to-mouse mutations into human CD40L (only 3 amino acids are different between human and mouse CD40L). We study human CD40L-mouse Fc and mouse CD40L-mouse Fc in animal models. This allows detection of the species-related differences in the CD40L decoy.

Myristic Acid Modification of CD40L Decoy

If necessary myristoylation is used. Albumin is the most abundant plasma protein involved in the transport of nutrients in the body, facilitated by its multiple binding sites and circulatory half-life of ˜19 days (49). Albumin contains multiple hydrophobic binding pockets (e.g., 5 myristic acid binding sites, Myr1-5) and naturally serves as a transporter of a variety of different ligands such as fatty acids and steroids as well as different drugs (49). The affinity of fatty acids such as myristic acid for human serum albumin with at least five binding sites is high with Kd values of ˜0.05 μM. For example, the C-terminal amino acid threonine in recombinant human insulin is replaced by a lysine moiety, and myristic acid is then covalently linked to its ε-amino group (Insulin Levemir). This chemically modified insulin has a long half-life, and one subcutaneous injection per day is sufficient to normalize the blood glucose level. We use free Cys-194 of CD40L, which is not involved in CD40 or integrin binding, to conjugate myristic acid using a sulfhydryl-reactive cross-linker (by incubating with myristic acid PEG maleimide, commercially available from Nanocos). Myristic (Myr) CD40L decoy will bind to serum mouse albumin when injected to mice. Our CD40L decoy is produced in E. coli and purified using Ni-NTA affinity chromatography and further purified by gel-filtration in FPLC. To remove endotoxin, we use recently developed endotoxin-free E. coli BL21 (commercially available). The CD40L decoy is endotoxin-free. We then inject Myr-CD40L decoy intraperitoneally and the protein quickly binds to serum albumin.

B) Study the Effect of CD40L Decoy in a Mouse Model of Atherosclerosis

The CD40 pathway is involved in inflammation and immune processes contributing to atherogenesis, and studies with neutralizing antibodies and CD40 deficient mice indicate that inhibiting this pathway is effective in reducing atherosclerotic lesions. Antibodies against CD40L in a mouse model consisting of LDL receptor deficient mice fed a high-cholesterol diet reduced the size and lipid content of early plaques, and the numbers of inflammatory cell infiltrates in plaques by T cells and macrophages were also reduced by antibody treatment, as well as marker VCAM1 (22). Further support for a crucial role of CD40L in atherosclerotic disease was reported in studies of CD40L deficient mice in the ApoE deficient background. Both early and advanced atherosclerotic plaque development were significantly impaired, inflammatory cell infiltrates were reduced, and other properties contributing to plaque pathology were also diminished (23). These studies support therapeutic development of inhibitors targeting the CD40L pathway in atherosclerosis. Here, we replicate mouse studies of atherosclerosis disease in ApoE-deficient mice (JAX) maintained on a high-cholesterol diet as described above (23) because lesions developed more closely resemble those in the LDL fraction in human patients. We focus on the early changes in foamy monocytes in blood that that express CD11c⁺CD36⁺. In contrast, most nonfoamy monocytes express either CD11c⁻CD36⁻ or CD11c⁻ CD36⁺ as previously shown (48). In addition, CD11c⁺ (foamy) monocytes express higher levels of tumor necrosis factor α and interleukin IL-1β than do CD11c⁻ (nonfoamy) monocytes from apoE^{-/-} mice on WD. These early changes in blood monocytes predict the effect of CD40L decoy. FACS analysis will be performed. We use CD40L decoy that is stabilized by Fc fusion or myristoylation.

Administration of CD40L Decoy-Fc in Mouse Models

We use both male and female mice. ApoE^{-/-} mice are fed a normal chow until 8 weeks of age and then switched to western diet (WD, 21% fat, 0.15% cholesterol) for 1-5 weeks. CD40L decoy-Fc is injected intraperitoneally twice a week after switching to WD (Groups 1 and 2, n=25 each, at different Myr-CD40L doses). Control group 3 (n=25) receives vehicle only. Blood is collected every week after switching to WD (n=5) and the levels of foamy monocytes, and activation of integrin α4β1, CD11c expression on monocytes is monitored as markers for nascent atherosclerosis as described (47). The CD40L decoy-Fc will reduce the levels of these markers. For a long-term treatment, 8-week-old ApoE^{-/-} mice are fed WD and receive either CD40L decoy-Fc or vehicle by intraperitoneal injection twice a week (22). After 12 weeks of treatment, mice will are sacrificed and aortas analyzed. Tissue samples are stained for markers (e.g., CD11c, VCAM-1, and CD31) as described (48). Atheroma development and pathology are analyzed by measurements in aortic arch plaque dimensions, lipid content by histologic staining and infiltrating T cells and macrophages plaques by immunohistology. We use anti-mouse CD40L monoclonal antibodies (commercially available) as a positive control.

Statistical Analysis and Power Analysis

Levels of inflammation are compared across groups using standard repeated measures mixed models (50). These models allow for possibly unequal spacing of measurements or unequal lengths of follow-up, as, for example, if some mice develop unsustainable tumor burdens and are sacrificed early. We formulate these models to test specifically for CD40L decoy-treated mice vs control, then test for the added impact of increasing doses, to identify an optimal dose level, on the levels of inflammation. We found that power analysis in the previous in vivo experiments are such that a 20% difference between treatment and control groups can be detected with 8 mice in each group (51, 52). We thus typically use 10-12 mice per group, unless we have pilot data that suggests a much better than 20% effect, in which case we use 8 mice per group.

Effective CD40L decoy-Fc serves as a novel and effective agent and has advantages over other types of CD40 inhibitors because of higher specificity to cognate receptors over kinase inhibitors and has better penetrance into the diseased tissues than IgG (180K) because of their smaller sizes (<29K). The proposed experiments establish that CD40L decoy-Fc effectively suppresses disease progression.

CD40L decoy suppression of inflammatory and disease indicators in atherosclerosis includes suppressed upregulation of CD11c and CD34 on circulating monocytes, suppressed leukocyte recruitment to the atheroma, and reduced atherosclerotic lesions, without affecting humoral and cellular immune responses (including antibody production). The experiments described here establish that the CD40L-integrin interaction is a novel therapeutic target and that CD40L decoys are useful as therapeutic agents.

Effective CD40L decoys serve as novel and effective agents and have advantages over other types of CD40 inhibitors because of higher specificity for cognate receptors over kinase inhibitors and better penetrance into diseased tissues than IgG (180K) because of their smaller sizes (<29K). The experiments described herein establish that myristoylated CD40L decoys effectively suppress disease progression.

### Summary

The work described herein challenges the previous understanding of CD40L-CD40 signaling and establishes the role of integrins in CD40L signaling as a co-receptor. We develop CD40L decoys and evaluate their efficacy in vitro and in vivo. Antibodies specific to CD40L have been shown to be effective in reducing atherosclerosis in animal models, but the development of anti-CD40L was halted due to thromboembolic events in clinical trials. Other strategies to reduce CD40L-CD40 signaling have been sought. Our preliminary studies found that CD40L mutants defective in integrin binding are useful as antagonists to CD40L-CD40 signaling. Successful identification of antagonistic CD40L mutants leads to development of agents for treatment of atherosclerotic disease and other chronic inflammatory diseases.

### REFERENCES

1. Jones, J. I., T. Prevette, A. Gockerman, and D. R. Clemmons, Ligand occupancy of the alpha-V-beta3 integrin is necessary for smooth muscle cells to migrate in response to insulin-like growth factor. Proc Natl Acad Sci USA, 1996. 93(6): p. 2482-7.
2. Brooks, P. C., R. A. Clark, and D. A. Cheresh, Requirement of vascular integrin alpha v beta 3 for angiogenesis. Science, 1994. 264(5158): p. 569-71.
3. Kim, S. H., J. Turnbull, and S. Guimond, Extracellular matrix and cell signalling: the dynamic cooperation of integrin, proteoglycan and growth factor receptor. J Endocrinol, 2011. 209(2): p. 139-51. DOI: 10.1530/JOE-10-0377.
4. Odenthal, J., R. Takes, and P. Friedl, Plasticity of tumor cell invasion: governance by growth factors and cytokines. Carcinogenesis, 2016. 37(12): p. 1117-1128. DOI: 10.1093/carcin/bgw098.
5. Desgrosellier, J. S. and D. A. Cheresh, Integrins in cancer: biological implications and therapeutic opportunities. Nat Rev Cancer, 2010. 10(1): p. 9-22. DOI: 10.1038/nrc2748.

6. Mori, S., C. Y. Wu, S. Yamaji, J. Saegusa, B. Shi, Z. Ma, Y. Kuwabara, K. S. Lam, R. R. Isseroff, Y. K. Takada, and Y. Takada, Direct Binding of Integrin {alpha}v{beta}3 to FGF1 Plays a Role in FGF1 Signaling. J Biol Chem, 2008. 283(26): p. 18066-75. DOI: M801213200 [pii] 10.1074/jbc.M801213200.
7. Saegusa, J., S. Yamaji, K. Ieguchi, C. Y. Wu, K. S. Lam, F. T. Liu, Y. K. Takada, and Y. Takada, The direct binding of insulin-like growth factor-1 (IGF-1) to integrin alphavbeta3 is involved in IGF-1 signaling. J Biol Chem, 2009. 284(36): p. 24106-14. DOI: M109.013201 [pii] 10.1074/jbc.M109.013201.
8. Ieguchi, K., M. Fujita, Z. Ma, P. Davari, Y. Taniguchi, K. Sekiguchi, B. Wang, Y. K. Takada, and Y. Takada, Direct binding of the EGF-like domain of neuregulin-1 to integrins ({alpha}v{beta}3 and {alpha}6{beta}4) is involved in neuregulin-1/ErbB signaling. J Biol Chem, 2010. 285(41): p. 31388-98. DOI: M110.113878 [pii] 10.1074/jbc.M110.113878.
9. Yamaji, S., J. Saegusa, K. Ieguchi, M. Fujita, Y. K. Takada, and Y. Takada, A novel fibroblast growth factor-1 (FGF1) mutant that acts as an FGF antagonist. PLoS One, 2010. 5(4): p. e10273. DOI: 10.1371/journal.pone.0010273.
10. Mori, S., V. Tran, K. Nishikawa, T. Kaneda, Y. Hamada, N. Kawaguchi, M. Fujita, Y. K. Takada, N. Matsuura, M. Zhao, and Y. Takada, A Dominant-Negative FGF1 Mutant (the R50E Mutant) Suppresses Tumorigenesis and Angiogenesis. PLoS One, 2013. 8(2): p. e57927. DOI: 10.1371/journal.pone.0057927.
11. Fujita, M., K. Ieguchi, D. M. Cedano-Prieto, A. Fong, C. Wilkerson, J. Q. Chen, M. Wu, S. H. Lo, A. T. Cheung, M. D. Wilson, R. D. Cardiff, A. D. Borowsky, Y. K. Takada, and Y. Takada, An Integrin Binding-defective Mutant of Insulin-like Growth Factor-1 (R36E/R37E IGF1) Acts as a Dominant-negative Antagonist of the IGF1 Receptor (IGF1R) and Suppresses Tumorigenesis but Still Binds to IGF1R. J Biol Chem, 2013. 288(27): p. 19593-603. DOI: 10.1074/jbc.M113.470872.
12. Fujita, M., Y. K. Takada, and Y. Takada, Integrins alphavbeta3 and alpha4beta1 Act as Coreceptors for Fractalkine, and the Integrin-Binding Defective Mutant of Fractalkine Is an Antagonist of CX3CR1. J Immunol, 2012. 189(12): p. 5809-5819. DOI: 10.4049/jimmunol.1200889.
13. Takada, Y. K., J. Yu, M. Fujita, J. Saegusa, C. Y. Wu, and Y. Takada, Direct binding to integrins and loss of disulfide linkage in interleukin-1beta (IL-1beta) are involved in the agonistic action of IL-1beta. J Biol Chem, 2017. 292(49): p. 20067-20075. DOI: 10.1074/jbc.M117.818302.
14. Grewal, I. S. and R. A. Flavell, CD40 and CD154 in cell-mediated immunity. Annu Rev Immunol, 1998. 16: p. 111-35. DOI: 10.1146/annurev.immunol.16.1.111.
15. Chatzigeorgiou, A., M. Lyberi, G. Chatzilymperis, A. Nezos, and E. Kamper, CD40/CD40L signaling and its implication in health and disease. Biofactors, 2009. 35(6): p. 474-83. DOI: 10.1002/biof.62.
16. Schonbeck, U. and P. Libby, The CD40/CD154 receptor/ligand dyad. Cell Mol Life Sci, 2001. 58(1): p. 4-43.
17. Cha, J. K., M. H. Jeong, H. R. Bae, J. Y. Han, S. J. Jeong, H. J. Jin, Y. J. Lim, S. H. Kim, and J. W. Kim, Activated platelets induce secretion of interleukin-1beta, monocyte chemotactic protein-1, and macrophage inflammatory protein-1alpha and surface expression of intercellular adhesion molecule-1 on cultured endothelial cells. J Korean Med Sci, 2000. 15(3): p. 273-8. DOI: 10.3346/jkms.2000.15.3.273.
18. Daub, K., H. Langer, P. Seizer, K. Stellos, A. E. May, P. Goyal, B. Bigalke, T. Schonberger, T. Geisler, D. Siegel-Axel, R. A. Oostendorp, S. Lindemann, and M. Gawaz, Platelets induce differentiation of human CD34+ progenitor cells into foam cells and endothelial cells. FASEB J, 2006. 20(14): p. 2559-61. DOI: 10.1096/fj.06-6265fie.
19. Henn, V., J. R. Slupsky, M. Grafe, I. Anagnostopoulos, R. Forster, G. Muller-Berghaus, and R. A. Kroczek, CD40 ligand on activated platelets triggers an inflammatory reaction of endothelial cells. Nature, 1998. 391(6667): p. 591-4. DOI: 10.1038/35393.
20. Crow, M. K. and K. A. Kirou, Regulation of CD40 ligand expression in systemic lupus erythematosus. Curr Opin Rheumatol, 2001. 13(5): p. 361-9.
21. Delmas, Y., J. F. Viallard, A. Solanilla, J. Villeneuve, J. M. Pasquet, F. Belloc, I. Dubus, J. Dechanet-Merville, P. Merville, P. Blanco, J. L. Pellegrin, A. T. Nurden, C. Combe, and J. Ripoche, Activation of mesangial cells by platelets in systemic lupus erythematosus via a CD154-dependent induction of CD40. Kidney Int, 2005. 68(5): p. 2068-78. DOI: 10.1111/j.1523-1755.2005.00663.x.
22. Mach, F., U. Schonbeck, G. K. Sukhova, E. Atkinson, and P. Libby, Reduction of atherosclerosis in mice by inhibition of CD40 signalling. Nature, 1998. 394(6689): p. 200-3. DOI: 10.1038/28204.
23. Lutgens, E., L. Gorelik, M. J. Daemen, E. D. de Muinck, I. S. Grewal, V. E. Koteliansky, and R. A. Flavell, Requirement for CD154 in the progression of atherosclerosis. Nat Med, 1999. 5(11): p. 1313-6. DOI: 10.1038/15271.
24. Alaaeddine, N., G. S. Hassan, D. Yacoub, and W. Mourad, CD154: an immunoinflammatory mediator in systemic lupus erythematosus and rheumatoid arthritis. Clin Dev Immunol, 2012. 2012: p. 490148. DOI: 10.1155/2012/490148.
25. Hassan, G. S., J. Stagg, and W. Mourad, Role of CD154 in cancer pathogenesis and immunotherapy. Cancer Treat Rev, 2015. 41(5): p. 431-40. DOI: 10.1016/j.ctrv.2015.03.007.
26. Hassan, G. S., Y. Merhi, and W. Mourad, CD40 ligand: a neo-inflammatory molecule in vascular diseases. Immunobiology, 2012. 217(5): p. 521-32. DOI: 10.1016/j.imbio.2011.03.010.
27. Andre, P., K. S. Prasad, C. V. Denis, M. He, J. M. Papalia, R. O. Hynes, D. R. Phillips, and D. D. Wagner, CD40L stabilizes arterial thrombi by a beta3 integrin—dependent mechanism. Nat Med, 2002. 8(3): p. 247-52. DOI: 10.1038/nm0302-247.
28. El Fakhry, Y., H. Alturaihi, D. Yacoub, L. Liu, W. Guo, C. Leveille, D. Jung, L. B. Khzam, Y. Merhi, J. A. Wilkins, H. Li, and W. Mourad, Functional interaction of CD154 protein with alpha5beta1 integrin is totally independent from its binding to alphaIIbbeta3 integrin and CD40 molecules. J Biol Chem, 2012. 287(22): p. 18055-66. DOI: 10.1074/jbc.M111.333989.
29. Saegusa, J., N. Akakura, C. Y. Wu, C. Hoogland, Z. Ma, K. S. Lam, F. T. Liu, Y. K. Takada, and Y. Takada, Pro-inflammatory secretory phospholipase A2 type IIA binds to integrins alphavbeta3 and alpha4beta1 and induces proliferation of monocytic cells in an integrin-dependent manner. J Biol Chem, 2008. 283(38): p. 26107-15. DOI: M804835200 [pii] 10.1074/jbc.M804835200.
30. Fujita, M., Y. K. Takada, and Y. Takada, The chemokine fractalkine can activate integrins without CX3CR1 through direct binding to a ligand-binding site distinct from the classical RGD-binding site. PLoS One, 2015. 9(5): p. e96372. DOI: 10.1371/journal.pone.0096372.
31. Fujita, M., K. Zhu, C. K. Fujita, M. Zhao, K. S. Lam, M. J. Kurth, Y. K. Takada, and Y. Takada, Proinflammatory secreted phospholipase A2 type IIA (sPLA-IIA) induces integrin activation through direct binding to a newly identified binding site (site 2) in integrins alphavbeta3, alpha4beta1, and alpha5beta1. J Biol Chem, 2015. 290(1): p. 259-71. DOI: 10.1074/jbc.M114.579946.
32. Fujita, M., P. Davari, Y. K. Takada, and Y. Takada, Stromal cell-derived factor-1 (CXCL12) activates integrins by direct binding to an allosteric ligand-binding site (site 2) of integrins without CXCR4. Biochem J, 2018. DOI: 10.1042/BCJ20170867.
33. Singh, J., E. Garber, H. Van Vlijmen, M. Karpusas, Y. M. Hsu, Z. Zheng, J. H. Naismith, and D. Thomas, The role of polar interactions in the molecular recognition of CD40L with its receptor CD40. Protein Sci, 1998. 7(5): p. 1124-35. DOI: 10.1002/pro.5560070506.
34. Alturaihi, H., G. S. Hassan, L. Al-Zoobi, S. Salti, Y. Darif, D. Yacoub, S. El Akoum, M. Oudghiri, Y. Merhi, and W. Mourad, Interaction of CD154 with different receptors and its role in bidirectional signals. Eur J Immunol, 2015. 45(2): p. 592-602. DOI: 10.1002/eji.201444941.
35. Leveille, C., M. Bouillon, W. Guo, J. Bolduc, E. Sharif-Askari, Y. El-Fakhry, C. Reyes-Moreno, R. Lapointe, Y. Merhi, J. A. Wilkins, and W. Mourad, CD40 ligand binds to alpha5beta1 integrin and triggers cell signaling. J Biol Chem, 2007. 282(8): p. 5143-51. DOI: 10.1074/jbc.M608342200.
36. Pietravalle, F., S. Lecoanet-Henchoz, J. P. Aubry, G. Elson, J. Y. Bonnefoy, and J. F. Gauchat, Cleavage of membrane-bound CD40 ligand is not required for inducing B cell proliferation and differentiation. Eur J Immunol, 1996. 26(3): p. 725-8. DOI: 10.1002/eji.1830260333.
37. Seyama, K., S. Nonoyama, I. Gangsaas, D. Hollenbaugh, H. F. Pabst, A. Aruffo, and H. D. Ochs, Mutations of the CD40 ligand gene and its effect on CD40 ligand expression in patients with X-linked hyper IgM syndrome. Blood, 1998. 92(7): p. 2421-34.
38. Katz, F., S. Hinshelwood, P. Rutland, A. Jones, C. Kinnon, and G. Morgan, Mutation analysis in CD40 ligand deficiency leading to X-linked hypogammaglobulinemia with hyper IgM syndrome. Hum Mutat, 1996. 8(3): p. 223-8. DOI: 10.1002/(SICI)1098-1004(1996)8:3&lt;223::AID-HUMU5&gt;3.0.CO;2-A.
39. Allen, R. C., R. J. Armitage, M. E. Conley, H. Rosenblatt, N. A. Jenkins, N. G. Copeland, M. A. Bedell, S. Edelhoff, C. M. Disteche, D. K. Simoneaux, and et al., CD40 ligand gene defects responsible for X-linked hyper-IgM syndrome. Science, 1993. 259(5097): p. 990-3.
40. Bachsais, M., N. Naddaf, D. Yacoub, S. Salti, N. Alaaeddine, F. Aoudjit, G. S. Hassan, and W. Mourad, The Interaction of CD154 with the alpha5beta1 Integrin Inhibits Fas-Induced T Cell Death. PLoS One, 2016. 11(7): p. e0158987. DOI: 10.1371/journal.pone.0158987.
41. Armitage, R. J., W. C. Fanslow, L. Strockbine, T. A. Sato, K. N. Clifford, B. M. Macduff, D. M. Anderson, S. D. Gimpel, T. Davis-Smith, C. R. Maliszewski, and et al., Molecular and biological characterization of a murine ligand for CD40. Nature, 1992. 357(6373): p. 80-2. DOI: 10.1038/357080a0.
42. LaFlamme, S. E., L. A. Thomas, S. S. Yamada, and K. M. Yamada, Single subunit chimeric integrins as mimics and inhibitors of endogenous integrin functions in receptor localization, cell spreading and migration, and matrix assembly. J Cell Biol, 1994. 126(5): p. 1287-98.
43. Legate, K. R. and R. Fassler, Mechanisms that regulate adaptor binding to beta-integrin cytoplasmic tails. Journal of cell science, 2009. 122(Pt 2): p. 187-98. DOI: 10.1242/jcs.041624.
44. Gong, H., B. Shen, P. Flevaris, C. Chow, S. C. Lam, T. A. Voyno-Yasenetskaya, T. Kozasa, and X. Du, G protein subunit Galpha13 binds to integrin alphaIIbbeta3 and mediates integrin “outside-in” signaling. Science, 2010. 327(5963): p. 340-3. DOI: 10.1126/science.1174779.
45. Shen, B., X. Zhao, K. A. O'Brien, A. Stojanovic-Terpo, M. K. Delaney, K. Kim, J. Cho, S. C. Lam, and X. Du, A directional switch of integrin signalling and a new anti-thrombotic strategy. Nature, 2013. 503(7474): p. 131-5. DOI: 10.1038/nature12613.
46. Lossner, D., C. Abou-Ajram, A. Benge, and U. Reuning, Integrin alphavbeta3 mediates upregulation of epidermal growth-factor receptor expression and activity in human ovarian cancer cells. Int J Biochem Cell Biol, 2008. 40(12): p. 2746-61. DOI: 51357-2725(08)00215-X [pii] 10.1016/j.bioce1.2008.05.014.
47. Foster, G. A., L. Xu, A. A. Chidambaram, S. R. Soderberg, E. J. Armstrong, H. Wu, and S. I. Simon, CD11c/CD18 Signals Very Late Antigen-4 Activation To Initiate Foamy Monocyte Recruitment during the Onset of Hypercholesterolemia. J Immunol, 2015. 195(11): p. 5380-92. DOI: 10.4049/jimmunol.1501077.
48. Xu, L., X. Dai Perrard, J. L. Perrard, D. Yang, X. Xiao, B. B. Teng, S. I. Simon, C. M. Ballantyne, and H. Wu, Foamy monocytes form early and contribute to nascent atherosclerosis in mice with hypercholesterolemia. Arterioscler Thromb Vasc Biol, 2015. 35(8): p. 1787-97. DOI: 10.1161/ATVBAHA.115.305609.
49. Larsen, M. T., M. Kuhlmann, M. L. Hvam, and K. A. Howard, Albumin-based drug delivery: harnessing nature to cure disease. Mol Cell Ther, 2016. 4: p. 3. DOI: 10.1186/s40591-016-0048-8.
50. Laird, N. M. and J. H. Ware, Random-effects models for longitudinal data. Biometrics, 1982. 38(4): p. 963-74.
51. Namba, R., L. J. Young, C. K. Abbey, L. Kim, P. Damonte, A. D. Borowsky, J. Qi, C. G. Tepper, C. L. MacLeod, R. D. Cardiff, and J. P. Gregg, Rapamycin inhibits growth of premalignant and malignant mammary lesions in a mouse model of ductal carcinoma in situ. Clin Cancer Res, 2006. 12(8): p. 2613-21.
52. Namba, R., L. J. Young, J. E. Maglione, E. T. McGoldrick, S. Liu, G. T. Wurz, M. W. DeGregorio, A. D. Borowsky, C. L. MacLeod, R. D. Cardiff, and J. P. Gregg, Selective estrogen receptor modulators inhibit growth and progression of premalignant lesions in a mouse model of ductal carcinoma in situ. Breast Cancer Res, 2005. 7(6): p. R881-9. DOI: bcr1317 [pii] 10.1186/bcr1317.

Key resources: CHO cells that express recombinant integrins, Ramos human B-cell line from ATCC, and antibodies that recognize human integrins (e.g., β3, β1).

### Vectors

1. CD40L cDNAs (WT and mutant) in pCDNA3 were obtained from ATCC.

2. Fc expression vectors (pFUSE-hIgG1-Fc, pFUSE-mIgG1-Fc) from Invivogen.

### Cells

CHO cells that express recombinant integrins were previously generated in our lab. They have been extensively tested in many laboratories including ours and published.

We perform short tandem repeat profiling using vendors (e.g., ATCC and Genetica) to identify the cell lines and to avoid cross-contamination. We test mycoplasma once per year using commercial kits. We keep cells in culture at most 1 month, discard them, and wake up new batch from frozen vials to avoid genetic drift.

We test mycoplasma infection of cells periodically using commercially available kits. If positive we treat them using antibiotics.

### Proteins

1. Proteins are mostly prepared in our lab in E. coli and purified as soluble protein using affinity chromatography. They are >90% pure in SDS-gel electrophoresis. We check the size (by SDS-PAGE and staining) after synthesis. If protein size needs to be confirmed we re-sequence the expression plasmids. We validate the function of our WT CD40L using commercial CD40L.

2. CD40 decoy-Fc is expressed in mammalian cells.

### Antibodies

Antibodies are obtained commercially (e.g., Santa Cruz biotechnology).

### Mice

We study both males and females. Mice are obtained from commercial sources such as The Jackson Laboratory or Charles River Laboratories.

### Mouse Model of Atherosclerosis

Two models of atherosclerosis are available in gene targeted mice based on the ApoE, and these models are available at Jackson Laboratories. Based on previous work, the ApoE/C57BL/6 deficient mice appear to more closely resemble human disease, which is why we use this model for our studies. The in vivo atherosclerosis models have been well characterized and are well suited for these experiments. Mice are used since in vivo inflammation models have been well established in mice.

### Narrative

We discovered that integrins bind to cytokines and form a triplex structure composed of integrin-cytokine-receptor (ternary complex model). Among the cytokines we studied is CD40L, which is involved, e.g., in inflammatory diseases. Our preliminary data demonstrated that we can use the ternary complex model to study CD40L/CD40 signaling, and to design new CD40L antagonists that are useful as novel drugs to treat inflammatory diseases (e.g., atherosclerosis) and other diseases.

### Example 3. Integrins αvβ3 and α5β1 Bind to the Trimeric Interface of CD40L, in which Genetic Mutations in High-IgM Syndrome Type 1 are Clustered

### Abstract

CD40 ligand (CD40L) plays a major role in immune response and is a major therapeutic target for inflammation. CD40L can bind to integrin α5β1 and CD40 simultaneously, but the role of integrins in CD40/CD40L signaling has previously been unclear. Genetic mutations in CD40L result in high-IgM syndrome type 1 (HIGMS1). Here we describe that integrin Δvβ3 is a new receptor for CD40L. Docking simulation predicted that the integrin αvβ3 binding site is located in the trimeric interface of CD40L. Mutating the predicted integrin-binding site suppressed the binding to integrin αvβ3 and integrin α5β1, indicating that they bind to monomeric CD40L. The CD40L mutants defective in integrin binding were defective in anti-apoptotic action, while they still bound to CD40. Interestingly, the CD40L mutants defective in integrin binding suppressed anti-apoptotic action induced by WT CD40L, indicating that the CD40L mutants are antagonistic. Also, several HIGMS1 mutations are located in the predicted integrin-binding site in trimeric interface and reduced integrin binding to CD40L. These findings presented here indicate that: 1) integrins Δvβ3 and α5β1 bind to monomeric CD40L, 2) CD40L signaling requires integrin binding, 3) the CD40L mutants defective in integrin-binding are antagonistic, and 4) genetic mutations in HIGMS1 in the trimeric interface lead to reduced integrin binding and result in functional defects in CD40L. We show that CD40L-integrin interaction is a new therapeutic target for inflammation.

### Significance

CD40L binds to several integrins including α5β1, but their role in CD40L signaling has previously been unclear. We identified integrin αvβ3 as a new CD40L receptor and located the integrin Δvβ3/α5β1 binding sites in the trimeric interface of CD40L monomer, which is cryptic in trimeric CD40L, indicating that integrins bind only to monomeric CD40L. The CD40L mutants defective in integrin binding were defective in signaling and were antagonistic, while they still bound to CD40, indicating that CD40L/CD40 signaling requires integrin binding. Also, HIGMS1 mutations are clustered in the predicted integrin-binding site, indicating that HIGMS1 mutations in CD40L lead to reduced integrin binding and functional defects. We propose that CD40L mutants defective in integrin have utility as a therapeutic in inflammation and cancer.

### Introduction

CD40 is a cell surface receptor that belongs to the tumor necrosis factor receptor (TNFR) family, which was first identified and functionally characterized on B lymphocytes. That CD40 plays a critical role in T cell-dependent humoral immune responses is demonstrated by patients with the X-linked hyper-IgM syndrome (HIGMS1), in which CD40 ligand (CD40L) is defective (1), and by gene targeting CD40 in mice. However, CD40 is expressed much more broadly, including expression on monocytes, dendritic cells, endothelial cells, and epithelial cells. In addition, the CD40 ligand (CD40L/CD154), a member of the TNF family, is also expressed more widely than activated CD4+ T cells only. Therefore, it is now understood that CD40-CD40L interactions play a more general role in immune regulation.

Previous reports indicate that integrins αIIbβ3 (2), α5β1 (3), and αMβ2 (4) bind to CD40L in addition to CD40. Each of these integrins interacts with CD40L in a specific manner. Integrin αIIbβ3 recognizes the KGD motif at the N-terminus of CD40L (residues 115-117 of CD40L). In contrast, integrin α5β1 does not require the KGD motif. Mutations of the CD40-binding site (Y145A, R203A, or Y145A/R203A double mutant) did not affect the integrin α5β1-CD40L interaction (5), suggesting that integrin α5β1 and CD40 can co-exist on CD40L. It has recently been reported that the N151A/Q166 double mutation suppresses integrin α5β1-mediated CD40L binding (6).

The integrin α5β1-mediated CD40L signaling has been characterized in malignant T cell leukemia cells (7). The binding of CD40L to integrin α5β1 induces anti-apoptotic signals, promotes survival of malignant T cell leukemia, and facilitates tumor development and propagation. CD40L binding to integrin α5β1 activates key survival proteins such as p38 and ERK1/2 mitogen activated protein kinases (MAPKs), phopsphoinositide 3 kinase (PI-3K), and Akt. Soluble CD40L significantly inhibits Fas-mediated apoptosis in T cell leukemia-lymphoma cell lines, an important hallmark of T cell survival during malignancy progression. The CD40L-triggered inhibition of the Fas-mediated cell death response is dependent on the suppression of caspase-8 cleavage (7). However, the specifics of integrin-CD40L interaction were not fully established and the role of integrins in CD40L signaling has been largely ignored.

In this example, we show that CD40L bound specifically to integrin Δvβ3 as a new CD40L receptor. We also show that integrin Δvβ3 and integrin α5β1 binding sites are located in the trimerization interface of CD40L. Mutating amino acid residues in the predicted integrin-binding site at the trimeric interface reduced the binding of CD40L to integrin Δvβ3 and integrin α5β1. This indicates that monomeric CD40L binds to these integrins and induces anti-apoptotic signals. It has been reported that some of the altered residues in HIGMS1 are located in the protein core or trimerization interface, pointing to roles in structural stability or trimer formation (8). Notably, we found that several HIGMS1 mutations at the trimeric interface suppressed integrin binding, indicating that defective integrin binding to CD40L is at least in part related to defective CD40L function in HIGMS1.

### Results

Integrin αvβ3 Binds to CD40L in a KGD-Independent Manner

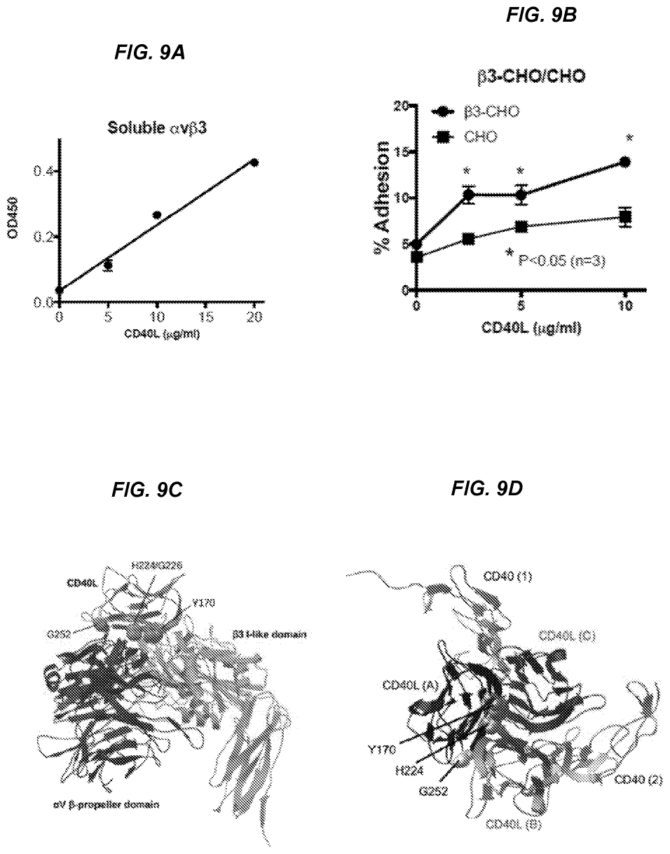

It has been reported that soluble CD40L (sCD40L) bound to immobilized soluble integrin α5β1 but did not bind to soluble integrin Δvβ3 (3). However, the reported binding experiments were performed in PBS without cations (3), so it is possible that integrin Δvβ3 did not bind to CD40L because it was not fully activated in these conditions. We thus studied if soluble integrin Δvβ3 that was fully activated by 1 mM Mn²⁺ bound to CD40L in ELISA-type binding assays. We used sCD40L (i.e., residues 118-261) that has no KGD motif at the N-terminus (i.e., residues 115-117 are not present). We found that integrin Δvβ3 bound to WT sCD40L in a dose-dependent manner (FIG. 9A). These findings show that integrin Δvβ3 specifically binds to CD40L receptor in a KGD-independent manner. We found that CD40L supports adhesion of CHO cells that express recombinant Δvβ3 (β3-CHO cells, Δvβ3+, α5β1+) better than parent CHO cells (α5β1+) (FIG. 9B), indicating that CD40L binds to transmembrane integrins. However, antibody 7E3 (specific to β3) or cRGDfV that was specific to integrin Δvβ3 did not inhibit the binding of Δvβ3 to CD40L.

### The Integrin Binding Site is Located in the Trimerization Interface

We studied how CD40L binds to integrin Δvβ3 using a simulation of docking between monomeric CD40L that lacks the KGD motif (PDB code 1ALY) and the headpiece of integrin Δvβ3 (PDB code 1L5G) (FIG. 9C). 20 out of 100 dockings grouped in the first cluster (docking energy −24.5 kcal/mol). The simulation predicted that monomeric CD40L binds to the RGD-binding site of integrin Δvβ3. Notably, the integrin binding site in CD40L was predicted to be located in the trimerization interface (FIG. 9D). The amino acid residues in the predicted integrin Δvβ3-CD40L interaction are shown in Table 1. It has been reported that the N151A/Q166A mutation on the surface of trimeric CD40L is involved in integrin α5β1 binding (6), but these amino acid residues are not involved in the integrin binding interface. The KGD motif in CD40L is not involved in the integrin binding interface, consistent with the idea that CD40L binds to integrin Δvβ3 in a KGD-independent manner. Based on the prediction, we selected several amino acid residues within the predicted integrin binding site for mutagenesis.

TABLE 1
Amino acid residues within 0.6 nm between CD40L and αvβ3 as
selected using pdb viewer (version 4.1). Amino acid residues
in CD40L that are mutated in HIGM1 are shown in bold.

| CD40L | αv (1L5G) | β3 (1L5G) |
|---|---|---|
| Leu168, Tyr170, Tyr172, | Tyr80, Glu117, Met118, | Tyr122, Ser123, Lys125, |
| Gln174, Thr176, Leu205, | Lys119, Gln145, Asp146, | Asp126, Asp127, Tyr166, |
| Leu206, Arg207, Ala208, | Ile147, Asp148, Ala149, | Tyr178, Asp179, Met180, |
| Ala209, Asn210, Gln220, | Asp150, Gly151, Tyr178, | Lys181, Thr182, Arg214, |
| Gln221, Ser222, Ile223, | Arg211, Thr212, ala213, | Arg216, Asp251, Ala252, |
| His224, Leu225, Gly226, | Gln214, Ala215, Ile216, | Lys253, Thr311, Asn313, |
| Gly227, Val228, Phe229, | Phe217, Asp218, Arg248, | Val314 |
| Glu230, Thr251, Gly252, | | |
| Phe253, Ser255, Leu258, | | |
| Leu259, Lys260, Leu261 | | |

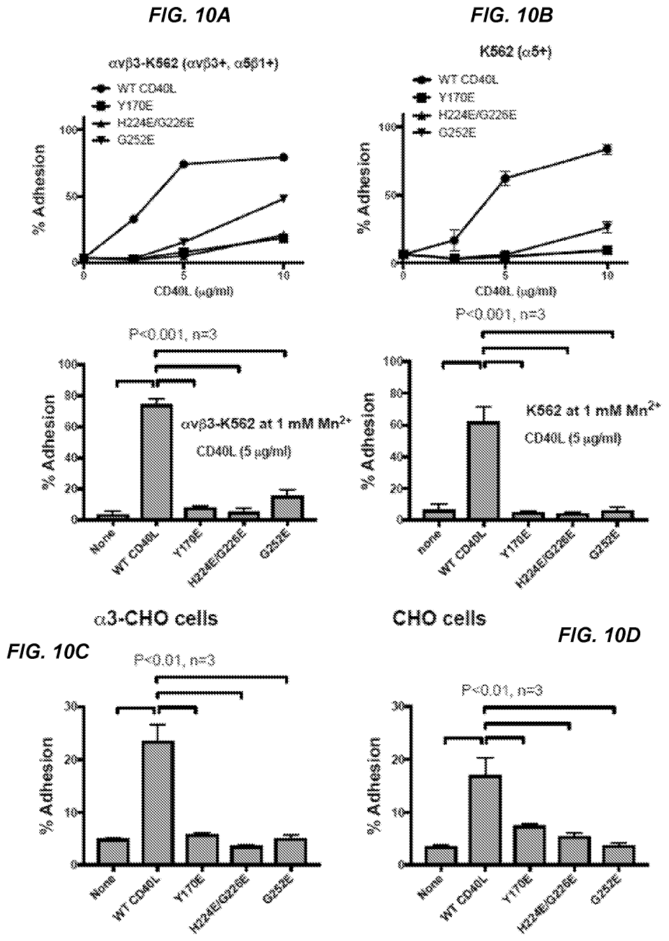

We found that Y170E, H224E/G226E, and G252E mutations significantly reduced adhesion of K562 cells that expressed recombinant integrin Δvβ3 (Δvβ3-K562) (FIG. 10A) and parent K562 cells (α5β1+) (FIG. 10B). In addition, we obtained very similar results using CHO cells that expressed recombinant Δvβ3 (β3-CHO cells, Δvβ3+, α5β1+) (FIG. 10C) and parent CHO cells (α5β1+) (FIG. 10D). These findings show that the critical amino acid residues for integrin Δvβ3 binding are located in the trimeric interface of CD40L.

We also studied whether the CD40L mutations affect integrin α5β1 binding in adhesion assays using K562 cells (α5β1+, Δvβ3−) that do not express CD40 (9). In the presence of 1 mM Mn²⁺, K562 cells adhered well to wild-type (wt) CD40L. We obtained similar results using β3-CHO cells (CD40-negative) (FIGS. 10C and 10D). These findings show that the CD40L mutants were defective in binding to integrin α5β1 or integrin Δvβ3. These results also indicate that the integrin Δvβ3 and integrin α5β1 binding sites overlap. These results, which are consistent with the docking model, indicate that integrin Δvβ3 and integrin α5β1 bind to monomeric CD40L through binding sites that are cryptic in a CD40L trimer. The present results are also consistent with the previous report that integrin α5β1 does not require the KGD motif (6).

### The CD40L Mutants Defective in Integrin Binding are Defective in Inducing Proliferation of Ramos Cells and Suppress Cell Proliferation Induced by WT CD40L

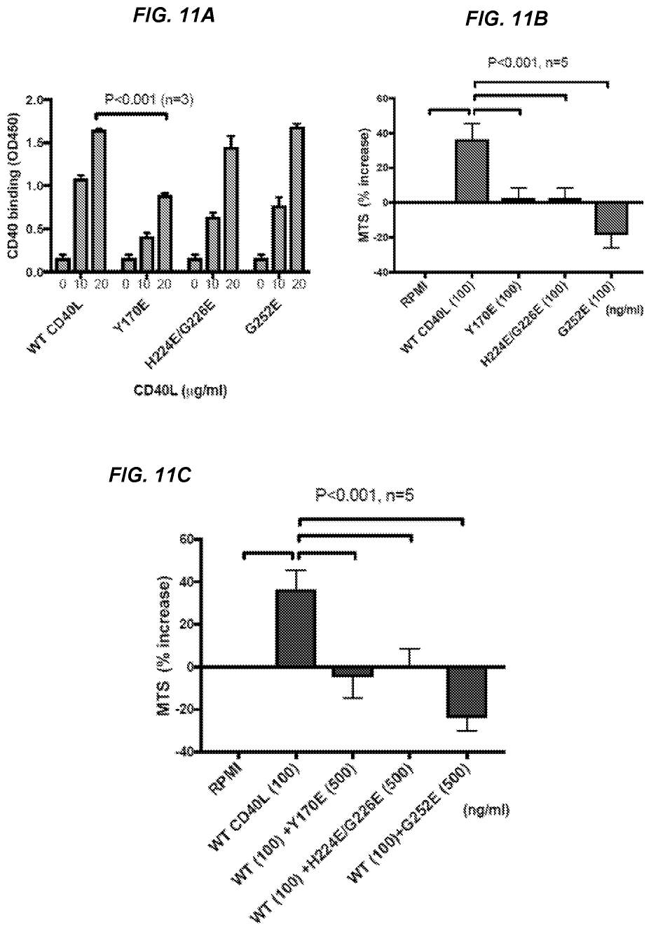

We found that CD40L mutants that were defective in integrin binding still bound to CD40 in ELISA-type binding assays, except that Y170E showed lower binding to CD40. This indicates that they are properly folded and also that the integrin binding and CD40 binding sites are distinct (FIG. 11A). Previous studies showed that CD40L binding to integrin α5β1 induced anti-apoptotic signals (3, 5, 7, 10). We found that the CD40L mutants which were defective in integrin binding were also defective in inducing proliferation of Ramos B-cell lymphoma cells (FIG. 11B). Notably, the mutants suppressed cell proliferation that was induced by WT CD40L, demonstrating that the CD40L mutants were dominant negative antagonists (FIG. 11C). This is consistent with the idea that CD40L signaling requires both CD40 binding and integrin binding.

### Several HIGMS1 Mutants are Defective in Integrin Binding

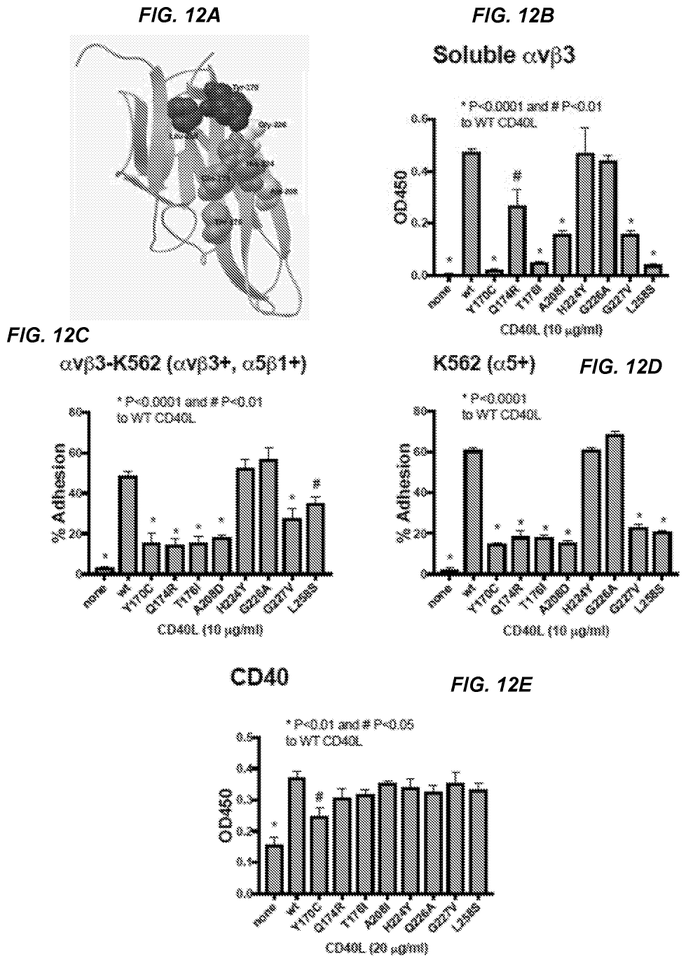

It has been reported that somatic mutations in CD40L result in HIGMS1 due to structural or functional defects in CD40L (1). Furthermore, it has been proposed that the CD40L mutations induce defects in the structure and trimerization of CD40L since they are mostly located in the trimeric interface. Interestingly, we noticed that several HIGMS1 mutations are located in the integrin-binding site within CD40L (Y170C (11), Q174R (12), T1761, A208D, H224Y, G226A, G227V (13), L258S (11), see, FIG. 12). We studied if several HIGMS1 mutations affected integrin binding to CD40L. We found that the HIGMS1 mutations Y170C, Q174R, T1761, G227V, and L258S reduced the binding of soluble integrin Δvβ3 (FIG. 12), but H224Y or G226A mutations did not (FIGS. 12C and 12D). Also, we obtained very similar results using K562 cells (α5β1+) and Δvβ3-K562 cells (Δvβ3+, α5β1+) in adhesion assays (FIGS. 12C and 12D). These results show that several HIGMS1 mutations affect integrin binding to CD40L. However, these mutations had little or no effect on the binding of CD40, except for Y170C (FIG. 12D). These findings indicate that several genetic mutations in the integrin binding site within the trimeric interface induce CD40L functional defects that lead to HIGMS1 due to reduced integrin binding.

### Discussion

This example establishes that integrin Δvβ3 is a new receptor for CD40L. Since integrin Δvβ3 is widely distributed in the vascular tissues and cancer, the interaction of CD40L with integrin Δvβ3 is biologically highly significant. Integrin Δvβ3 recognizes a binding site that is at the trimeric interface of CD40L, which is exposed in monomeric CD40L but not in trimeric CD40L. The CD40L mutations that affected binding to integrin Δvβ3 also affected binding to integrin α5β1, demonstrating that integrin Δvβ3 and integrin α5β1 share common binding sites. Based on the results presented in this example, we propose that CD40L monomer engages with integrins α5β1 and Δvβ3 and that their binding site is cryptic in trimeric CD40L. Therefore, it is expected that monomeric CD40L induces pro-inflammatory signals.

It has been understood that trimeric CD40L is biologically active, but that monomeric CD40L is not (14). Therefore, the role of monomeric CD40L in the pathogenesis of inflammatory diseases has been ignored. It has been reported that soluble CD40L levels markedly increase in systemic lupus erythematosus (SLE) (15, 16) and that most of the serum soluble CD40L was monomeric while only 15% appeared to be multimeric (17). Furthermore, the elevated plasma levels of soluble CD40L appear to be associated with autoimmune disease activity, and it has been proposed that this correlation defines a causal relationship (16). Similar to membrane-bound CD40L, elevated expression of sCD40L may contribute to immune activation of antigen-presenting cells and the stimulation of autoantibody-producing B cells in patients with SLE. In addition to its ability to activate B cells, monocytes, or dendritic cells, CD40L may also induce inflammatory changes in CD40-bearing non-hematopoietic cells. The reports of enhanced serum CD40L levels in SLE and other chronic inflammation may be directly related to the pro-inflammatory signals that are induced by monomeric CD40L. Therefore, it is important further explore the role of monomeric CD40L in CD40L signaling during chronic inflammation. For example, it is unclear whether CD40 and integrin Δvβ3 bind to CD40L simultaneously, resulting in the formation of a CD40-CD40L-integrin ternary complex, as in the case of integrin α5β1 (5).

Previous studies have shown that integrin antagonists inhibit insulin-like growth factor (IGF1) and fibroblast growth factor-2 (basic FGF, FGF2) signaling (18, 19). These findings indicate that integrins are involved in growth factor signaling through crosstalk. We have previously reported that several cytokines (FGF1, IGF1, neuregulin-1, and fractalkine) directly bind to integrins, and that these interactions lead to ternary complex formation (integrin-growth factor-cognate receptor), which is required for their signaling functions (20-26). Recently, we found that the direct binding of integrins to IL-1β plays a critical role in IL-1β signaling (27). This indicates that crosstalk between integrins and growth factor receptors through direct integrin binding is a common mechanism in growth factor signaling. Mutants that are defective in integrin binding to these growth factors are functionally defective and suppress signaling that is induced by WT growth factors (i.e., the mutants are dominant negative antagonists) (23-25).

We found that CD40L mutants of the present invention were defective in integrin binding and were also defective in inducing the proliferation of Ramos cells, although they still bound to CD40, demonstrating that CD40 binding was not sufficient and that CD40L required integrin binding for inducing signals. Notably, the CD40L mutants that were defective in integrin binding suppressed cell proliferation that was induced by WT CD40L, demonstrating that the mutants of the present invention were dominant negative. Without being bound to any particular theory, we believe that CD40L-integrin binding is required for CD40L/CD40 signaling, as in other forms of growth factor/cytokine signaling. It is likely that monomeric or dimeric CD40L induces such a complex. CD40L is a key player in chronic autoimmune inflammatory diseases, including systemic lupus erythematosus (SLE), diabetes, and chronic kidney disease (28, 29). Based on striking efficacy in preclinical models, clinical trials using humanized or chimeric anti-CD40L monoclonal antibodies that block CD40/CD40L interactions were undertaken in the early 2000s. However, progress was halted due to the incidence of thromboembolic events in clinical trials (it is generally accepted that thromboembolic complications may be a class effect of anti-CD40L mAbs independent of epitope specificity) (30). Thus, the dominant negative CD40L mutants of the present invention are useful as therapeutics (e.g., in inflammation).

We found that several HIGMS1-associated mutations are present in the predicted integrin binding site in the trimeric interface and that they showed reduced integrin binding while CD40 binding was not affected. It is likely that several HIGMS1 mutations affected integrin binding and thereby induced CD40L functional defects (defective Ig class switching). These findings in HIGMS1 mutants are consistent with the idea that direct integrin binding to monomeric CD40L plays a critical role in CD40L signaling. Integrin binding to monomeric CD40L is thus a new therapeutic target in inflammation and other pathological conditions in which CD40L is involved.

### Materials and Methods

### Materials

Recombinant soluble Δvβ3 was synthesized in Chinese hamster ovary (CHO) K1 cells using the soluble αv and β3 expression constructs and purified by nickel-nitrilotriacetic acid (Ni-NTA) affinity chromatography as previously described (31). CHO cells that express human β3 have been described (32). Cyclic RGDfV was purchased from Enzo Life Sciences (Farmingdale, N.Y.).

Synthesis of Recombinant Soluble CD40L with No KGD Motif

We synthesized a recombinant soluble CD40L fragment (residues 118-261, QNPQIAAHVISEASSKTTSVLQWAEKGYYTMSNNLVTLENGKQLTVKRQGLYYIYAQV TFCSNREASSQAPFIASLCLKSPGRFERILLRAANTHSSAKPCGQQSIELGGVFELQPGAS VFVNVTDPSQVSHGTGFTSFGLLKL; SEQ ID NO:2) with no N-terminal KGD motif. We amplified a cDNA fragment using primers 5′-cgggatcccagaatcctcaaattgcggcac-3′ (SEQ ID NO:3) and 5′-cggaattctcagagtttgagtaagccaaagg-3′ (SEQ ID NO:4) by PCR and subcloned it into the BamH1/EcoR1 fragment of PET28a. Protein was synthesized in E. coli BL21 induced by isopropyl β-D-thiogalactoside as an insoluble protein and purified by N-NTA affinity chromatography under denatured condition and refolded as described (21).

For the synthesis of CD40 fused to GST, the cDNA fragment encoding the CD40 fragment (residues 21-144) was amplified by PCR and subcloned into the BamHI/EcoRI site of PGEX2T. We synthesized the proteins in BL21 cells and purified using glutathione-sepharose affinity chromatography.

The CD40 fragment fused to GST was coated onto wells of a 96-well microtiter plate (100 μm/ml in PBS) for 1 hour, and the remaining protein-binding sites were blocked by BSA (0.1%). We then incubated the wells with sCD40L and incubated for 1 hour, and bound GST was measured using HRP-conjugated anti-GST.

For cell proliferation, Ramos cells (2×10⁴ cells/well) were serum-starved overnight in serum-free media (RPMI1640) and incubated with proteins (WT or mutant CD40L) for 24 hours in a 96-well plate. Cell proliferation was measured by MTS assay using an Aqueous cell proliferation assay kit (Promega, Madison, Wis.).

### Adhesion Assays

Adhesion assays were performed as described previously (21). Briefly, to assess cell adhesion to immobilized CD40L, 96-well Immulon-2 microtiter plates were coated with 100 μl of 0.1 M NaHCO₃ containing CD40L or its mutant and were incubated for 2 hours at 37° C. Remaining protein binding sites were blocked by incubating with PBS/0.1% BSA for 30 minutes at room temperature. After washing with PBS, K562 cells in 100 μl of RPMI 1640/0.1% BSA were added to the wells and incubated at 37° C. for 1 hour. After unbound cells were removed by rinsing the wells with the medium used for adhesion assays, bound cells were quantified by measuring endogenous phosphatase activity (21). To activate integrins, Hepes-Tyrodes buffer with 1 mM MnCl₂ was used instead of RPMI 1640.

### Docking Simulation

Docking simulation of interaction between CD40L (1ALY.pdb), which does not contain an N-terminal KGD motif, and integrin Δvβ3 was performed using AutoDock3 as described (21). In the present study, we used the headpiece (residues 1-438 of αv and residues 55-432 of β3) of integrin Δvβ3 (open-headpiece form, 1L5G.pdb). Cations were not present in Δvβ3 during docking simulation (20,32).

Treatment differences were tested using ANOVA and a Tukey multiple comparison test to control the global type I error using Prism 7 (Graphpad Software).

### REFERENCES

1. Piirila H, Valiaho J, & Vihinen M (2006) Immunodeficiency mutation databases (IDbases). Hum Mutat 27(12):1200-1208.
2. Andre P, et al. (2002) CD40L stabilizes arterial thrombi by a beta3 integrin—dependent mechanism. Nat Med 8(3):247-252.
3. Leveille C, et al. (2007) CD40 ligand binds to alpha5beta1 integrin and triggers cell signaling. J Blot Chem 282(8):5143-5151.

4. Zirlik A, et al. (2007) CD40 ligand mediates inflammation independently of CD40 by interaction with Mac-1. Circulation 115(12):1571-1580.
5. El Fakhry Y, et al. (2012) Functional interaction of CD154 protein with alpha5beta1 integrin is totally independent from its binding to alphaIIbbeta3 integrin and CD40 molecules. J Biol Chem 287(22): 18055-18066.
6. Alturaihi H, et al. (2015) Interaction of CD154 with different receptors and its role in bidirectional signals. Eur J Immunol 45(2):592-602.
7. Bachsais M, et al. (2016) The Interaction of CD154 with the alpha5beta1 Integrin Inhibits Fas-Induced T Cell Death. PLoS One 11(7):e0158987.
8. Karpusas M, et al. (1995) 2 A crystal structure of an extracellular fragment of human CD40 ligand. Structure 3(10):1031-1039.
9. Thomas W D, Smith M J, Si Z, & Hersey P (1996) Expression of the co-stimulatory molecule CD40 on melanoma cells. Int J Cancer 68(6):795-801.
10. El Fakhry Y, Alturaihi H, Diallo D, Merhi Y, & Mourad W (2010) Critical role of lipid rafts in CD154-mediated T cell signaling. Eur J Immunol 40(3):770-779.
11. Seyama K, et al. (1998) Mutations of the CD40 ligand gene and its effect on CD40 ligand expression in patients with X-linked hyper IgM syndrome. Blood 92(7):2421-2434.

12. Katz F, et al. (1996) Mutation analysis in CD40 ligand deficiency leading to X-linked hypogammaglobulinemia with hyper IgM syndrome. Hum Mutat 8(3):223-228.
13. Allen R C, et al. (1993) CD40 ligand gene defects responsible for X-linked hyper-IgM syndrome. Science 259(5097):990-993.
14. Pietravalle F, et al. (1996) Cleavage of membrane-bound CD40 ligand is not required for inducing B cell proliferation and differentiation. Eur J Immunol 26(3):725-728.
15. Vakkalanka R K, et al. (1999) Elevated levels and functional capacity of soluble CD40 ligand in systemic lupus erythematosus sera. Arthritis Rheum 42(5):871-881.
16. Kato K, et al. (1999) The soluble CD40 ligand sCD154 in systemic lupus erythematosus. J Clin Invest 104(7):947-955.
17. Alaaeddine N, Hassan G S, Yacoub D, & Mourad W (2012) CD154: an immunoinflammatory mediator in systemic lupus erythematosus and rheumatoid arthritis. Clin Dev Immunol 2012:490148.
18. Jones J I, Prevette T, Gockerman A, & Clemmons D R (1996) Ligand occupancy of the alpha-V-beta3 integrin is necessary for smooth muscle cells to migrate in response to insulin-like growth factor. Proc Natl Acad Sci USA 93 (6):2482-2487.
19. Brooks P C, Clark R A, & Cheresh D A (1994) Requirement of vascular integrin alpha v beta 3 for angiogenesis. Science 264(5158):569-571.
20. Mori S, et al. (2008) Direct Binding of Integrin {alpha}v{beta}3 to FGF1 Plays a Role in FGF1 Signaling. J Biol Chem 283(26):18066-18075.
21. Saegusa J, et al. (2009) The direct binding of insulin-like growth factor-1 (IGF-1) to integrin alphavbeta3 is involved in IGF-1 signaling. J Biol Chem 284(36):24106-24114.
22. Ieguchi K, et al. (2010) Direct binding of the EGF-like domain of neuregulin-1 to integrins ({alpha}v{beta}3 and {alpha}6{beta}4) is involved in neuregulin-1/ErbB signaling. J Biol Chem 285(41):31388-31398.
23. Yamaji S, et al. (2010) A novel fibroblast growth factor-1 (FGF1) mutant that acts as an FGF antagonist. PLoS One 5(4):e10273.
24. Mori S, et al. (2013) A Dominant-Negative FGF1 Mutant (the R50E Mutant) Suppresses Tumorigenesis and Angiogenesis. PLoS One 8(2):e57927.
25. Fujita M, et al. (2013) An Integrin Binding-defective Mutant of Insulin-like Growth Factor-1 (R36E/R37E IGF1) Acts as a Dominant-negative Antagonist of the IGF1 Receptor (IGF1R) and Suppresses Tumorigenesis but Still Binds to IGF1R. J Biol Chem 288(27):19593-19603.
26. Fujita M, Takada Y K, & Takada Y (2012) Integrins alphavbeta3 and alpha4beta1 Act as Coreceptors for Fractalkine, and the Integrin-Binding Defective Mutant of Fractalkine Is an Antagonist of CX3CR1. J Immunol 189(12):5809-5819.
27. Takada Y K, et al. (2017) Direct binding to integrins and loss of disulfide linkage in interleukin-1beta (IL-1beta) are involved in the agonistic action of IL-1beta. J Biol Chem 292(49):20067-20075.
28. Crow M K & Kirou K A (2001) Regulation of CD40 ligand expression in systemic lupus erythematosus. Curr Opin Rheumatol 13(5):361-369.
29. Delmas Y, et al. (2005) Activation of mesangial cells by platelets in systemic lupus erythematosus via a CD154-dependent induction of CD40. Kidney Int 68(5):2068-2078.
30. Zhang T, Pierson R N, 3rd, & Azimzadeh A M (2015) Update on CD40 and CD154 blockade in transplant models. Immunotherapy 7(8):899-911.
31. Takagi J, Erickson H P, & Springer T A (2001) C-terminal opening mimics ‘inside-out’ activation of integrin alpha5beta1. Nat Struct Biol 8(5):412-416.
32. Saegusa J, et al. (2008) Pro-inflammatory secretory phospholipase A2 type IIA binds to integrins alphavbeta3 and alpha4beta1 and induces proliferation of monocytic cells in an integrin-dependent manner. J Blot Chem 283(38):26107-26115.

### Example 4. Development of Antagonistic CD40L Mutants as Therapeutics for Chronic Inflammation

CD40 ligand (CD40L/CD154) plays key roles in immune regulation through its receptor CD40 and is a major target in inflammatory diseases. Previous work showed that antibodies to CD40L were effective in reducing inflammation and atherosclerosis in preclinical animal models, but their development was halted because of thrombotic incidents. Other possible ways to block this signaling pathway have been sought. The inventors of the present invention have found that CD40L binds to integrin Δvβ3 (a novel CD40L receptor), integrins are critically involved in CD40/CD40L signaling, and CD40L mutants of the present invention were defective in binding to Δvβ3 act as antagonists to CD40L. In this example, antagonistic CD40L mutants are stabilized by PEGylation or myristoylation and their efficacy is evaluated in mouse models of inflammation (psoriasis). The CD40L mutants can be used as antagonists of CD40L/CD40 signaling. If properly stabilized, they can be effectively used to reduce systemic inflammation (e.g., atherosclerosis, psoriasis, and SLE) and to facilitate transplantation.

### The Opportunity: Missing CD40L Antagonists

CD40 ligand (CD40L/CD154) plays key roles in immune regulation through its receptor CD40 by participating in antigen presentation and antigen recognition. CD40L is an identified major target in inflammatory diseases (1). CD40 is a cell surface receptor that belongs to the TNF receptor family and was first identified and functionally characterized on B lymphocytes. CD40 is now known to be expressed much more broadly. CD40-CD40L interactions play a more general role in immune regulation. CD40L-CD40 interaction is a key immunomodulatory factor responsible for modulating nearly all aspects of the adaptive immune response. CD40L is expressed as a transmembrane form and released as a soluble form (sCD40L) by proteolytic cleavage. CD40L/CD40 interaction is required for enhancing antigen presenting functions of dendritic cells, macrophages, and B cells; maturation of humoral responses; and enhancement of effector T cell responses. Additional functions of CD40L include the initiation of inflammatory and procoagulatory responses in vascular endothelial cells (2-4). CD40L is a key player in chronic autoimmune inflammatory diseases, including systemic lupus erythematosus (SLE) and psoriasis, diabetes, and chronic kidney disease, and is also involved in resistance to organ transplantation. CD40L levels markedly increase in certain pathologic conditions such as SLE and rheumatoid arthritis (1). Based on striking efficacy in preclinical models, clinical trials using humanized or chimeric anti-CD40L monoclonal antibodies blocking CD40/CD40L interactions were undertaken in early 2000s. Unfortunately, progress was halted due to the incidence of thromboembolic events in clinical trials. Alternative drugs have been explored but currently no CD40/CD40L antagonists are available in clinic. A new form of biologic therapeutic would, therefore, fill a significant niche.

### Novel Strategy to Develop Novel CD40L Antagonists

We recently discovered that integrins are common co-receptors of growth factor/chemokine signaling (ternary complex model). Growth factor/chemokine mutants defective in integrin binding are defective in signaling functions and act as antagonists (dominant-negative function). Since α5β1 and CD40 can simultaneously bind to CD40L (5), it is likely that they form a ternary complex. In silico analyses demonstrate that CD40L binds to integrins (e.g., α5β1) through a domain distinct from recognition by its cognate receptor CD40. These simulations suggest that CD40L can simultaneously bind to receptor CD40 and α5β1. Mutants prepared from protein structural modeling by selected amino acid changes confirmed the recognition of integrins by CD40L. This indicates that CD40L/CD40 signaling fits well with the ternary complex model. Additional studies undertaken with CD40L mutants suggest that CD40L signaling is mediated by ternary complex formation with integrin α5β1 and CD40, indicating that CD40L mutants defective in integrin binding act as antagonists to CD40L-mediated signaling. Such mutants are effective therapeutic agents for suppressing chronic inflammation. Additional data showed that CD40L also binds to another integrin Δvβ3 without affecting CD40 binding and that CD40L mutants did not bind to this integrin, supporting the concept that the ternary complex is biologically relevant. These findings were confirmed by inhibition of human B cell proliferation in vitro by co-culture with a CD40L mutant incapable of integrin binding, demonstrating that dominant-negative CD40L (dnCD40L) is a useful anti-inflammatory molecule. This example describes one way to test dnCD40L mutants in an inflammatory mouse model of psoriasis.

We have developed dnCD40L mutants that show efficacy in cell culture; the effect of dnCD40L mutants can be examined using a mouse model of psoriasis. The mouse model of psoriasis is induced by subcutaneous injections of IL-23. dnCD40L mutants are used to treat psoriasis by: 1) development of effective PEGylated dnCD40L, and 2) systemic delivery by i.p. injection of PEGylated dnCD40L. These studies provide proof of principle data on the utility of dnCD40L in a mouse model of psoriasis, and lead to optimization and preparation for large animal studies.

### Preliminary Results and Significance

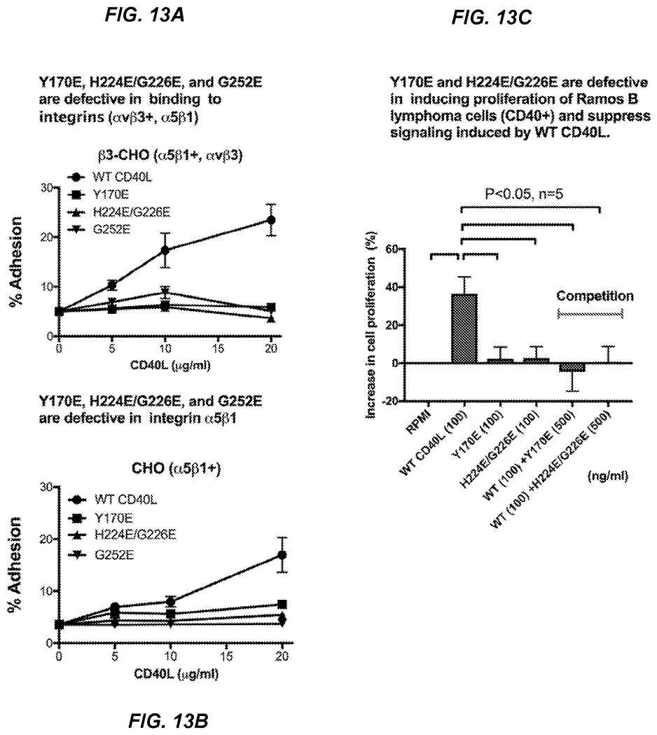

We have shown that CD40L binds to integrins α5β1 and Δvβ3 and this finding allowed us to perform detailed analysis of CD40L-integrin binding. We identified several CD40L mutations that suppress integrin binding in the trimeric interface (FIG. 1). These mutations were positioned at amino acid positions not in the CD40 binding site, the cognate receptor of CD40L. Additional studies confirmed that the dnCD40L mutants defective in integrin binding were indeed antagonistic to CD40L signaling, in characteristic dominant-negative fashion by inhibition of cell proliferation in human B lymphoma cells induced by wild-type CD40L (FIG. 13). This is consistent with the CD40L mutants having anti-inflammatory properties.

### Research Design

A mouse model of psoriasis induced by subcutaneous injection of IL-23 is widely used to study this disease and serves as the platform on which these studies are based.

Aim 1: PEGylation of dnCD40L

Unmodified dnCD40L is possibly rapidly eliminated in vivo. To address this, dnCD40L mutants are PEGylated to improve pharmacokinetic properties. PEGylation of biopharmaceuticals is a well-established strategy to stabilize proteins in vivo (6), and was also used in development of the anti-psoriatic drug certolizumab. Recently, PEGylated interleukin 2 was shown to be effective in immune therapy of 9 tumor types (e.g., melanoma and renal cell carcinoma). To accomplish PEGylation, a Cys residue is introduced at either the C- or N-terminus of dnCD40L. Conjugation is performed by reaction with commercially available maleimide-PEG. PEGylated dn-sCD40L (dnCD40L-PEG) is further purified by gel filtration in FPLC. ELISA is used to confirm that PEG conjugation to dn-sCD40L does not affecting CD40 binding. The half-life of PEG-CD40L is determined by labeling the dnCD40L-PEG (with I¹²⁵) and injecting it into mice. Blood samples are obtained and the levels of radioactivity are measured periodically. Tests are performed to confirm that dnCD40L-PEG has much better pharmacokinetics than unmodified dnCD40L and thus suitable for studies as therapeutic agents.

### Aim 2. Effect of Dn-sCD40L in Mouse Models of Inflammation

CD40-CD40L interactions are critical for effector T cell activation and are present on antigen-presenting cells (APCs) and T cells, respectively. APCs, including dendritic cells, macrophages and B cells, are T cell priming factors in skin inflammation, and these interactions are studied in skin models of inflammation. Psoriasis is a common skin disorder mediated by crosstalk between epidermal keratinocytes, dermal vascular cells, and immune cells. Daily injection of IL-23 in mouse skin triggers Th1 and Th17 cell-mediated adaptive immunity and mice rapidly exhibit thickened skin with erythema and scales resembling psoriasis. It has been reported that skin inflammation is significantly reduced in CD40-null mice compared with WT mice, which is accompanied by decreases in inflammatory cytokine production, indicating that this model is useful for evaluating the effects of dnCD40L peptides of the present invention. dn-sCD40L is locally administrated on the first day of IL-23 challenge. After a weeks' challenge, mice are euthanized and ears are collected for histology, immunohistochemistry, and flow cytometry, as well as testing for disease markers.

The dn-sCD40L inhibitors serve as novel and effective agents and demonstrate advantages over other types of CD40 inhibitors because of higher specificity for cognate receptors over kinase inhibitors. Undesired side effects of antibody inhibitors, such as crosslinking, are also avoided. This work improves longevity of dn-sCD40L-PEG in vivo, contributing to effective suppression of disease progression and contributing to disease reversal.

### REFERENCES

1. Alaaeddine, N., G. S. Hassan, D. Yacoub, and W. Mourad, CD154: an immunoinflammatory mediator in systemic lupus erythematosus and rheumatoid arthritis. Clin Dev Immunol, 2012. 2012: p. 490148. 10.1155/2012/490148
2. Cha, J. K., M. H. Jeong, H. R. Bae, J. Y. Han, S. J. Jeong, H. J. Jin, Y. J. Lim, S. H. Kim, and J. W. Kim, Activated platelets induce secretion of interleukin-1beta, monocyte chemotactic protein-1, and macrophage inflammatory protein-1alpha and surface expression of intercellular adhesion molecule-1 on cultured endothelial cells. J Korean Med Sci, 2000. 15(3): p. 273-8. 10.3346/jkms.2000.15.3.273
3. Daub, K., H. Langer, P. Seizer, K. Stellos, A. E. May, P. Goyal, B. Bigalke, T. Schonberger, T. Geisler, D. Siegel-Axel, R. A. Oostendorp, S. Lindemann, and M. Gawaz, Platelets induce differentiation of human CD34+ progenitor cells into foam cells and endothelial cells. FASEB J, 2006. 20(14): p. 2559-61. 10.1096/fj.06-6265fje
4. Henn, V., J. R. Slupsky, M. Grafe, I. Anagnostopoulos, R. Forster, G. Muller-Berghaus, and R. A. Kroczek, CD40 ligand on activated platelets triggers an inflammatory reaction of endothelial cells. Nature, 1998. 391(6667): p. 591-4. 10.1038/35393
5. El Fakhry, Y., H. Alturaihi, D. Yacoub, L. Liu, W. Guo, C. Leveille, D. Jung, L. B. Khzam, Y. Merhi, J. A. Wilkins, H. Li, and W. Mourad, Functional interaction of CD154 protein with alpha5beta1 integrin is totally independent from its binding to alphaIIbbeta3 integrin and CD40 molecules. J Biol Chem, 2012. 287(22): p. 18055-66. 10.1074/jbc.M111.333989

6. Bailon, P. and C. Y. Won, PEG-modified biopharmaceuticals. Expert Opin Drug Deliv, 2009. 6(1): p. 1-16. 10.1517/17425240802650568

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

1. An isolated polypeptide comprising the amino acid sequence of SEQ ID NO:1 in which at least one of Y170, H224, G226, and G252 is mutated.

2. The polypeptide of claim 1, which consists of the amino acid sequence of SEQ ID NO:1 in which at least one of Y170, H224, G226, and G252 is mutated.

3. The polypeptide of claim 1, wherein Y170 is mutated, preferably having the mutation of Y170E.

4. The polypeptide of claim 1, wherein H224 and G226 are mutated, preferably having the mutations of H224E and G226E.

5. The polypeptide of claim 1, wherein G252 is mutated, preferably having the mutation of G252E.

6. The polypeptide of claim 1, wherein the polypeptide suppresses CD40 signaling.

7. The polypeptide of claim 1, wherein the polypeptide binds to αvβ3 integrin or α5β1 integrin with a weaker affinity than a corresponding polypeptide that comprises the amino acid sequence of SEQ ID NO:1.

8. The polypeptide of claim 1, further comprising a cysteine residue at the N- and/or C-terminus of the amino acid sequence of SEQ ID NO:1.

9-10. (canceled)

11. A fusion protein comprising:
(a) a polypeptide comprising the amino acid sequence of SEQ ID NO:1 in which at least one of Y170, H224, G226, and G252 is mutated; and
(b) an Fc polypeptide.

12. The fusion protein of claim 11, wherein Y170 is mutated, preferably having the mutation of Y170E.

13. The fusion protein of claim 11, wherein H224 and G226 are mutated, preferably having the mutations of H224E and G226E.

14. The fusion protein of claim 11, wherein G252 is mutated, preferably having the mutation of G252E.

15-16. (canceled)

17. A composition comprising:
(a) the polypeptide of claim 1; and
(b) a physiologically acceptable carrier.

18. An isolated nucleic acid comprising a polynucleotide sequence that encodes the polypeptide of claim 1.

19. A method for suppressing CD40 signaling in a cell, comprising contacting the cell with an effective amount of the polypeptide of claim 1.

20. A method for inhibiting proliferation of a cell, comprising contacting the cell with an effective amount of the polypeptide of claim 1.

21. The method of claim 19 or 20, wherein the cell is a lymphocyte.

22. A method for preventing or treating an inflammatory or immune disorder or cancer in a subject in need thereof, comprising administering to the subject an effective amount of the polypeptide of claim 1.

23. The method of claim 22, wherein the inflammatory or immune disorder is selected from the group consisting of an autoimmune disorder, systemic lupus erythematosus (SLE), rheumatoid arthritis, atherosclerosis, psoriasis, diabetes, an inflammation- or immune-mediated renal disease, transplant rejection, and a combination thereof.

24. The method of claim 19, wherein a population of cells are contacted with the polypeptide, the composition, or the nucleic acid.

25-30. (canceled)
